SEQUENCE LISTING (47 sequences)

SEQ ID NO: 1 - protein, 16 aa, Clostridium botulinum
  PEPTIDE (0)...(0): N-terminal residues of mature, wild-type botulinum neurotoxin
PFVNKQFNYKDPVNGV

SEQ ID NO: 2 - protein, 17 aa, Human
  PEPTIDE (0)...(0): Residues 187-203 of SNAP-25
SNKTRIDEANQRATKML

SEQ ID NO: 3 - protein, 14 aa, Artificial Sequence
  Synthetic peptide; control for phosphorylation experiments
KVEKIGEGTGVVYK

SEQ ID NO: 4 - DNA, 1403 bp, Artificial Sequence
  Synthetic botulinum neurotoxin light chain of serotype A based on wild-type Clostridium botulinum sequence
gaattcccatggttcagttcgttaacaaacagttcaactacaaagacccggttaacggtgttgacatcgcttacatcaaaatcccgaacgttggtcagatgcagccggttaaagcattcaaaatccacaacaaaatctgggttatcccggaacgtgacactttcactaacccggaagaaggtgacctgaacccgccgccggaagctaaacaggttccggtttcttactacgactctacttacctgtctactgacaacgaaaaggacaactacctgaaaggtgttactaaactgtttgaacgtatctactctactgacctgggtcgcatgctgctcacttctatcgttcgtggtatcccgttctggggtggttctactatcgacactgaactgaaagttatcgacactaactgcatcaacgttatccagccggacggttcttaccgttctgaagaactgaacctggttatcatcggtccgtctgctgacatcatccagtttgaatgcaaatctttcggtcacgaagttctgaacctgactcgtaacggttacggttctactcagtacatccgtttctctccggacttcactttcggtttcgaagaatctctggaagttgacactaacccgctgctgggtgctggtaaattcgctactgacccggctgttactctggctcacgaactgatccacgctggtcaccgtctgtacggtatcgctatcaacccgaaccgtgttttcaaagttaacactaacgcttactacgaaatgtctggtctggaagtttcttttgaagaactgcgtactttcggtggtcacgacgctaaattcatcgactctctgcaggaaaacgagttccgtctgtactactacaacaaattcaaagacatcgcttctactctgaacaaagctaaatctatcgttggtaccactgcttctctgcagtacatgaagaacgttttcaaagaaaagtacctgctgtctgaagacacttctggtaaattctctgttgacaaactgaaattcgacaaactgtacaaaatgctgactgaaatctacactgaagacaacttcgttaaattcttcaaagttctgaaccgtaaaacttacctgaacttcgacaaagctgttttcaaaatcaacatcgttccgaaagttaactacactatctacgacggtttcaacctgcgtaacactaacctggctgctaacttcaacggtcagaacactgaaatcaacaacatgaacttcactaaactgaagaacttcactggtctgtttgagttctacaaactgctgtgcgttcgtggtatcatcacttctaaaactaaatctctggacaaaggttacaacaaactggttccgcgtggttctcatcatcatcatcatcattaatgagaatcc

SEQ ID NO: 5 - protein, 461 aa, Artificial Sequence
  Synthetic botulinum neurotoxin light chain of serotype A based on wild-type Clostridium botulinum sequence
MVQFVNKQFNYKDPVNGVDIAYIKIPNVGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKLVPRGSHHHHHH

SEQ ID NO: 6 - DNA, 1323 bp, Artificial Sequence
  Synthetic botulinum neurotoxin light chain of serotype B based on wild-type Clostridium botulinum sequence
atgccagttactattaacaacttcaactacaacgacccaattgacaacaacaacattattatgatggagccaccattcgctagaggtactggtagatactacaaggctttcaagattactgacagaatttggattattccagagagatacactttcggttacaagccagaggacttcaacaagtcttctggtattttcaacagagacgtttgtgagtactacgacccagactacttgaacactaacgacaagaagaacattttcttgcaaactatgattaagttgttcaacagaattaagtctaagccattgggtgagaagttgttggagatgattattaacggtattccatacttgggtgacagaagagttccattggaggagttcaacactaacattgcttctgttactgttaacaagttgatttctaacccaggtgaggttgagagaaagaagggtattttcgctaacttgattattttcggtccaggtccagttttgaacgagaacgagactattgacattggtattcaaaaccacttcgcttctagagagggtttcggtggtattatgcaaatgaagttctgtccagagtacgtttctgttttcaacaacgttcaagagaacaagggtgcttctattttcaacagaagaggttacttctctgacccagctttgattttgatgcacgagttgattcacgttttgcacggtttgtacggtattaaggttgacgacttgccaattgttccaaacgagaagaagttcttcatgcaatctactgacgctattcaagctgaggagttgtacactttcggtggtcaagacccatctattattactccatctactgacaagtctatttacgacaaggttttgcaaaacttcagaggtattgttgacagattgaacaaggttttggtttgtatttctgacccaaacattaacattaacatttacaagaacaagttcaaggacaagtacaagttcgttgaggactctgagggtaagtactctattgacgttgagtctttcgacaagttgtacaagtctttgatgttcggtttcactgagactaacattgctgagaactacaagattaagactagagcttcttacttctctgactctttgccaccagttaagattaagaacttgttggacaacgagatttacactattgaggagggtttcaacatttctgacaaggacatggagaaggagtacagaggtcaaaacaaggctattaacaagcaagcttacgaggagatttctaaggagcacttggctgtttacaagattcaaatgtgtaagtctgttaag

SEQ ID NO: 7 - protein, 441 aa, Artificial Sequence
  Synthetic botulinum neurotoxin light chain of serotype B based on wild-type Clostridium botulinum sequence
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVK

SEQ ID NO: 8 - DNA, 1332 bp, Artificial Sequence
  Synthetic botulinum neurotoxin light chain of serotype C1 based on wild-type Clostridium botulinum sequence
atgccaatcaccatcaacaacttcaactactcagaccctgtcgacaacaagaacattctgtacctggacactcacctgaacaccctagctaacgagcctgagaaggcctttcggatcaccggaaacatctgggtcatccctgatcgtttctcccgtaactccaaccccaacctgaacaagcctcctcgggtcaccagccctaagagtggttactacgaccctaactacctgagtaccgactctgacaaggacaccttcctgaaggagatcatcaagctgttcaagcgtatcaactcccgtgagatcggagaggagctcatctacagactttcgaccgatatccccttccctggtaacaacaatactccaatcaacaccttcgacttcgacgtcgacttcaactccgtcgacgtcaagactcggcagggtaacaactgggttaagactggtagcatcaacccttccgtcatcatcactggacctcgtgagaacatcatcgacccagagacttccacgttcaagctgactaacaacaccttcgcggctcaagaaggattcggtgctctgtcaatcatctccatctcacctcgtttcatgctgacctactcgaacgcaaccaacgacgtcggagagggtaggttctctaagtctgagttctgcatggacccaatcctgatcctgatgcatgagctgaaccatgcaatgcacaacctgtacggaatcgctatcccaaacgaccagaccatctcctccgtgacctccaacatcttctactcccagtacaacgtgaagctggagtacgcagagatctacgctttcggaggtccaactatcgaccttatccctaagtccgctaggaagtacttcgaggagaaggctttggattactacagatccatcgctaagagactgaacagtatcaccaccgcaaacccttccagcttcaacaagtacatcggtgagtacaagcagaagctgatcagaaagtaccgtttcgtcgtcgagtcttcaggtgaggtcacagtaaaccgtaacaagttcgtcgagctgtacaacgagcttacccagatcttcacagagttcaactacgctaagatctacaacgtccagaacaggaagatctacctgtccaacgtgtacactccggtgacggcgaacatcctggacgacaacgtctacgacatccagaacggattcaacatccctaagtccaacctgaacgtactattcatgggtcaaaacctgtctcgaaacccagcactgcgtaaggtcaaccctgagaacatgctgtacctgttcaccaagttctgccacaaggcaatcgacggtaga

SEQ ID NO: 9 - protein, 444 aa, Artificial Sequence
  Synthetic botulinum neurotoxin light chain of serotype C1 based on wild-type Clostridium botulinum sequence
MPITINNFNYSDPVDNKNILYLDTHLNTLANEPEKAFRITGNIWVIPDRFSRNSNPNLNKPPRVTSPKSGYYDPNYLSTDSDKDTFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGR

SEQ ID NO: 10 - DNA, 1323 bp, Artificial Sequence
  Synthetic botulinum neurotoxin light chain of serotype D based on wild-type Clostridium botulinum sequence
atgacctggccagtcaaggacttcaactactccgacccagtcaacgacaacgacatcttgtacttgagaatcccacaaaacaagttgatcaccaccccagtcaaggctttcatgatcacccagaacacctgggttatcccagagagattctcctccgacaccaacccatccctgtccaagccaccaagaccaacctccaagtaccagtcttactacgacccatcttacttgtctaccgacgagcaaaaggacaccttcttgaagggtattatcaagctgttcaagagaatcaacgagagagacatcggtaagaagttgatcaactacttggtcgttggttccccattcatgggtgactcctctaccccagaggacaccttcgacttcaccagacacaccaccaacattgccgtcgagaagttcgagaacggttcctggaaggtcaccaacatcatcaccccatctgttttgatcttcggtccattgccaaacatcttggactacaccgcctccctgaccttgcaaggtcagcaatccaacccatccttcgagggtttcggtaccctgtctattttgaaggtcgctccagagttcttgttgaccttctccgacgtcacctccaaccaatcctccgccgtcttgggtaagtccatcttctgtatggacccagtcatcgctttgatgcacgagttgacccactccctgcaccagttgtacggtattaacatcccatctgacaagagaatcagaccacaggtctctgagggtttcttctcccaagacggtccaaacgttcagttcgaggagttgtacaccttcggtggtttggacgtcgagattatccaaattgagagatcccaattgagagagaaggctttgggtcactacaaggacatcgccaagagactgaacaacatcaacaagaccattccatcttcctggatctccaacattgacaagtacaagaagattttctccgagaagtacaacttcgacaaggacaacaccggtaacttcgtcgttaacatcgacaagttcaactctttgtactccgacttgaccaacgttatgtctgaggttgtctactcctcccaatacaacgtcaagaacagaacccactacttctccagacactacttgccagttttcgctaacatcttggacgacaacatttacaccatcagagacggtttcaacttgaccaacaagggtttcaacatcgagaactccggtcaaaacatcgagagaaacccagccctgcaaaagctgtcctccgagtctgtcgtcgacttgttcaccaaggtctgtttgagattgaccaag

SEQ ID NO: 11 - protein, 441 aa, Artificial Sequence
  Synthetic botulinum neurotoxin light chain of serotype D based on wild-type Clostridium botulinum sequence
MTWPVKDFNYSDPVNDNDILYLRIPQNKLITTPVKAFMITQNTWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTK

SEQ ID NO: 12 - DNA, 1266 bp, Artificial Sequence
  Synthetic botulinum neurotoxin light chain of serotype E based on wild-type Clostridium botulinum sequence
atgccaaagattaactccttcaactacaacgaccctgtcaacgacagaaccatcttgtacatcaagccaggcggttgccaggagttctacaagtccttcaacatcatgaagaacatctggatcatccccgagagaaacgtcattggtaccaccccccaagacttccacccccctacttccttgaagaacggagactccagttactacgaccctaactacttgcaaagtgacgaggagaaggacagattcttgaagatcgtcacaaagatcttcaacagaatcaacaacaacctttcaggaggcatcttgttggaggagctgtccaaggctaacccatacttgggcaacgacaacactccagataaccagttccacattggtgacgcatccgcagttgagattaagttctccaacggtagccaggacatcctattgcctaacgttatcatcatgggagcagagcctgacttgtttgagaccaactcctccaacatctctctacgtaacaactacatgccaagcaatcacggtttcggatccatcgctatcgtcaccttctcccctgaatattccttcaggttcaacgacaacagcatgaacgagttcattcaggatcctgctctcacgctgatgcacgaattgatccactccttacatggactatatggcgctaagggcattactaccaagtacactatcacacagaagcagaaccccctaataaccaacatccggggtaccaacatcgaggagttcttgactttcggaggtactgacttgaacatcattactagtgctcagtccaacgacatctacactaaccttctggctgactacaagaagatcgcgtctaagcttagcaaggtccaagtctctaacccactgcttaacccttacaaggacgtcttcgaagcaaagtatggattggacaaggatgctagcggaatttactcggtcaacatcaacaagttcaacgacatcttcaagaagctctacagcttcacggagttcgacttggccaccaagttccaggttaagtgtaggcagacttacatcggacagtacaagtacttcaagctgtccaacctgttgaacgactctatctacaacatctcagaaggctacaacatcaacaacttgaaggtcaacttcagaggacagaatgcaaacttgaaccctagaatcattaccccaatcaccggtagaggactggtcaagaagatcatccgtttctgcaagaacattgtctctgtcaagggcatcagg

SEQ ID NO: 13 - protein, 422 aa, Artificial Sequence
  Synthetic botulinum neurotoxin light chain of serotype E based on wild-type Clostridium botulinum sequence
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIR

SEQ ID NO: 14 - DNA, 1308 bp, Artificial Sequence
  Synthetic botulinum neurotoxin light chain of serotype F based on wild-type Clostridium botulinum sequence
atgccagtcgctatcaactccttcaactacaacgacccagtcaacgacgacaccattttgtacatgcagatcccatacgaggagaagtctaagaagtactacaaggctttcgagatcatgagaaacgtctggattatcgagagaaacaccatcggtaccaacccatccgacttcgacccaccagcctctttgaagaacggttcctccgcttactacgacccaaactacttgaccaccgacgccgagaaggacagatacttgaagaccaccatcaagttgttcaagagaattaactctaacccagccggtaaggtcttgttgcaagagatctcctacgctaagccatacctgggtaacgaccacaccccaattgacgagttctccccagtcaccagaaccacctccgtcaacatcaagtctaccaacgttgagtcctccatgttgttgaacttgttggttctgggtgctggtccagacattttcgagtcttgttgttacccagtcagaaagctgatcgacccagacgttgtttacgacccatctaactacggtttcggttccattaacatcgttaccttctctccagagtacgagtacaccttcaacgacatctccggtggtcacaactcctccaccgagtctttcattgctgacccagccatctccctggctcacgagctgattcacgctttgcacggtttgtacggtgctagaggtgtcacctacgaggagaccattgaggtcaagcaagccccattgatgatcgccgagaagccaatcagattggaggagttcttgaccttcggtggtcaggacttgaacatcatcacctccgctatgaaggagaagatctacaacaacctgctggccaactacgagaagattgccaccagattgtccgaggtcaactctgccccaccagagtacgacatcaacgagtacaaggactacttccaatggaagtacggtttggacaagaacgccgacggttcctacaccgtcaacgagaacaagtccaacgagatttacaagaagttgtactctttcaccgagtccgacctggctaacaagttcaaggttaagtgtagaaacacctacttcatcaagtacgagttcttgaaggttccaaacctgttggacgacgacatctacaccgtttctgagggtttcaacatcggtaacttggctgtcaacaacagaggtcagtccattaagctgaacccaaagatcattgactccccagacaagggtctggttgagaagattgtcaagttctgtaagtccgtcatcccaagaaagggtaccaag

SEQ ID NO: 15 - protein, 436 aa, Artificial Sequence
  Synthetic botulinum neurotoxin light chain of serotype F based on wild-type Clostridium botulinum sequence
MPVAINSFNYNDPVNDDTILYMQIPYEEKSKKYYKAFEIMRNVWIIERNTIGTNPSDFDPPASLKNGSSAYYDPNYLTTDAEKDRYLKTTIKLFKRINSNPAGKVLLQEISYAKPYLGNDHTPIDEFSPVTRTTSVNIKSTNVESSMLLNLLVLGAGPDIFESCCYPVRKLIDPDVVYDPSNYGFGSINIVTFSPEYEYTFNDISGGHNSSTESFIADPAISLAHELIHALHGLYGARGVTYEETIEVKQAPLMIAEKPIRLEEFLTFGGQDLNIITSAMKEKIYNNLLANYEKIATRLSEVNSAPPEYDINEYKDYFQWKYGLDKNADGSYTVNENKSNEIYKKLYSFTESDLANKFKVKCRNTYFIKYEFLKVPNLLDDDIYTVSEGFNIGNLAVNNRGQSIKLNPKIIDSPDKGLVEKIVKFCKSVIPRKGTK

SEQ ID NO: 16 - DNA, 1317 bp, Artificial Sequence
  Synthetic botulinum neurotoxin light chain of serotype G based on wild-type Clostridium botulinum sequence
atgccagtcaacatcaagaacttcaactacaacgacccaattaacaacgacgacatcatgatggagccattcaacgacccaggtccaggtacctactacaaggctttcagaatcattgacagaatttggatcgttccagagagattcacctacggtttccaaccagaccagttcaacgcctccaccggtgtcttctctaaggacgtctacgagtactacgacccaacctacttgaagaccgacgctgagaaggacaagttcttgaagaccatgatcaagttgttcaacagaattaactctaagccatccggtcaaagattgttggacatgattgttgacgctattccatacttgggtaacgcctccaccccaccagacaagttcgctgccaacgtcgctaacgtttctatcaacaagaagattatccaaccaggtgctgaggaccagatcaagggtttgatgaccaacttgattattttcggtccaggtccagtcttgtccgacaacttcaccgactctatgatcatgaacggtcactccccaatttccgagggtttcggtgctagaatgatgatcagattctgtccatcctgtttgaacgttttcaacaacgtccaagagaacaaggacacctctatcttctctagaagagcttacttcgctgacccagctctgaccctgatgcacgagttgatccacgtcttgcacggtctgtacggtattaagatctccaacctgccaattaccccaaacaccaaggagttcttcatgcaacactccgacccagttcaagccgaggagctgtacaccttcggtggtcacgacccatctgtttccccatctaccgacatgaacatttacaacaaggctctgcagaacttccaagacattgctaacagactgaacatcgtctcctctgcccaaggttctggtatcgacatttccttgtacaagcaaatctacaagaacaagtacgacttcgtcgaggacccaaacggtaagtactctgttgacaaggacaagttcgacaagctgtacaaggctttgatgttcggtttcaccgagaccaacttggccggtgagtacggtattaagaccagatactcttacttctctgagtacctgccaccaatcaagaccgagaagttgttggacaacaccatctacacccagaacgagggtttcaacattgcttccaagaacttgaagaacgagttcaacggtcagaacaaggccgtcaacaaggaggcctacgaggagatttccctggagcacttggtcatctacagaatcgctatgtgtaagccagtcatgtac

SEQ ID NO: 17 - protein, 439 aa, Artificial Sequence
  Synthetic botulinum neurotoxin light chain of serotype G based on wild-type Clostridium botulinum sequence
MPVNIKNFNYNDPINNDDIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVSPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKNEFNGQNKAVNKEAYEEISLEHLVIYRIAMCKPVMY

SEQ ID NO: 18 - DNA, 1239 bp, Artificial Sequence
  Synthetic N-terminal region of the heavy chain of botulinum neurotoxin serotype A based on wild-typeClostridium botulinum sequence
atggctctgaacgacctgtgcatcaaagttaacaactgggacctgttcttctccccgtctgaagacaacttcactaacgacctgaacaaaggcgaagaaatcacctccgacactaacatcgaagctgctgaagaaaacatctctctggacctgatccagcagtactacctgactttcaacttcgacaacgaaccggaaaacatctccatcgaaaacctgtcttccgacatcatcggtcagctggaactgatgccgaacatcgaacgcttcccgaacggcaagaaatacgaactggacaaatacaccatgttccactacctgcgtgctcaggaattcgaacacggtaaatctcgtatcgctctgactaactccgttaacgaagctctgctgaacccgtctcgcgtttacaccttcttctcttccgactacgttaagaaagttaacaaagctactgaagctgctatgttcctgggttgggttgaacagctggtttacgacttcaccgacgaaacttctgaagtttccaccactgacaaaatcgctgacatcactatcatcatcccgtacatcggcccggctctgaacatcggtaacatgctgtacaaagacgacttcgttggtgctctgatcttctctggcgctgttatcctgctggaattcatcccggaaatcgctatcccggttctgggtaccttcgctctggtttcctacatcgctaacaaagttctgactgttcagaccatcgacaacgctctgtctaaacgtaacgaaaaatgggacgaagtttacaaatacatcgttactaactggctggctaaagttaacactcagatcgacctgatccgtaagaagatgaaagaagctctggaaaaccaggctgaagctactaaagctatcatcaactaccagtacaaccagtacaccgaagaagaaaagaacaacatcaacttcaacatcgatgacctgtcctctaaactgaacgaatccatcaacaaagctatgatcaacatcaacaaattcctgaaccagtgctctgtttcctacctgatgaactctatgatcccgtacggcgttaaacgcctggaagacttcgacgcttccctgaaagacgctctgctgaaatacatccgtgacaactacggtactctgatcggccaggttgaccgtctgaaagacaaggttaacaacaccctgtctactgacatcccgttccagctgtccaaatacgttgacaaccag

SEQ ID NO: 19 - protein, 413 aa, Artificial Sequence
  Synthetic N-terminal region of the heavy chain of botulinum neurotoxin serotype A based on wild-typeClostridium botulinum sequence
MALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIRDNYGTLIGQVDRLKDKVNNTLSTDIPFQLSKYVDNQ

SEQ ID NO: 20 - DNA, 2583 bp, Artificial Sequence
  Synthetic polynucleotide sequence for the light chain with Hn of C. botulinum Type A.
atggttcagttcgttaacaaacagttcaactacaaagacccggttaacggtgttgacatcgcttacatcaaaatcccgaacgttggtcagatgcagccggttaaagcattcaaaatccacaacaaaatctgggttatcccggaacgtgacactttcactaacccggaagaaggtgacctgaacccgccgccggaagctaaacaggttccggtttcttactacgactctacttacctgtctactgacaacgaaaaggacaactacctgaaaggtgttactaaactgtttgaacgtatctactctactgacctgggtcgcatgctgctcacttctatcgttcgtggtatcccgttctggggtggttctactatcgacactgaactgaaagttatcgacactaactgcatcaacgttatccagccggacggttcttaccgttctgaagaactgaacctggttatcatcggtccgtctgctgacatcatccagtttgaatgcaaatctttcggtcacgaagttctgaacctgactcgtaacggttacggttctactcagtacatccgtttctctccggacttcactttcggtttcgaagaatctctggaagttgacactaacccgctgctgggtgctggtaaattcgctactgacccggctgttactctggctcacgaactgatccacgctggtcaccgtctgtacggtatcgctatcaacccgaaccgtgttttcaaagttaacactaacgcttactacgaaatgtctggtctggaagtttcttttgaagaactgcgtactttcggtggtcacgacgctaaattcatcgactctctgcaggaaaacgagttccgtctgtactactactacaaattcaaagacatcgcttctactctgaacaaagctaaatctatcgttggtaccactgcttctctgcagtacatgaagaacgttttcaaagaaaagtacctgctgtctgaagacacttctggtaaattctctgttgacaaactgaaattcgacaaactgtacaaaatgctgactgaaatctacactgaagacaacttcgttaaattcttcaaagttctgaaccgtaaaacttacctgaacttcgacaaagctgttttcaaaatcaacatcgttccgaaagttaactacactatctacgacggtttcaacctgcgtaacactaacctggctgctaacttcaacggtcagaacactgaaatcaacaacatgaacttcactaaactgaagaacttcactggtctgtttgagttctacaaactgctgtgcgttcgtggtatcatcacttctaaaactaaatctctggacaaaggttacaacaaagctctgaacgacctgtgcatcaaagttaacaactgggacctgttcttctccccgtctgaagacaacttcactaacgacctgaacaaaggcgaagaaatcacctccgacactaacatcgaagctgctgaagaaaacatctctctggacctgatccagcagtactacctgactttcaacttcgacaacgaaccggaaaacatctccatcgaaaacctgtcttccgacatcatcggtcagctggaactgatgccgaacatcgaacgcttcccgaacggcaagaaatacgaactggacaaatacaccatgttccactacctgcgtgctcaggaattcgaacacggtaaatctcgtatcgctctgactaactccgttaacgaagctctgctgaacccgtctcgcgtttacaccttcttctcttccgactacgttaagaaagttaacaaagctactgaagctgctatgttcctgggttgggttgaacagctggtttacgacttcaccgacgaaacttctgaagtttccaccactgacaaaatcgctgacatcactatcatcatcccgtacatcggcccggctctgaacatcggtaacatgctgtacaaagacgacttcgttggtgctctgatcttctctggcgctgttatcctgctggaattcatcccggaaatcgctatcccggttctgggtaccttcgctctggtttcctacatcgctaacaaagttctgactgttcagaccatcgacaacgctctgtctaaacgtaacgaaaaatgggacgaagtttacaaatacatcgttactaactggctggctaaagttaacactcagatcgacctgatccgtaagaagatgaaagaagctctggaaaaccaggctgaagctactaaagctatcatcaactaccagtacaaccagtacaccgaagaagaaaagaacaacatcaacttcaacatcgatgacctgtcctctaaactgaacgaatccatcaacaaagctatgatcaacatcaacaaattcctgaaccagtgctctgtttcctacctgatgaactctatgatcccgtacggcgttaaacgcctggaagacttcgacgcttccctgaaagacgctctgctgaaatacatccgtgacaactacggtactctgatcggccaggttgaccgtctgaaagacaaggttaacaacaccctgtctactgacatcccgttccagctgtccaaatacgttgacaaccag

SEQ ID NO: 21 - protein, 861 aa, Artificial Sequence
  Recombinant protein encoded by SEQ ID NO20
MVQFVNKQFNYKDPVNGVDIAYIKIPNVGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYYKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIRDNYGTLIGQVDRLKDKVNNTLSTDIPFQLSKYVDNQ

SEQ ID NO: 22 - DNA, 1329 bp, Artificial Sequence
  Synthetic polynucleotide sequence for the light chain of C. botulinum Type B, optimized for expression in E. coli.
atgccagttaccatcaacaacttcaactacaacgacccaatcgacaacaacaacatcattatgatggagccaccattcgctagaggtaccggtagatactacaaggctttcaagatcaccgacagaatttggattattccagagagatacactttcggttacaagccagaggacttcaacaagtcttctggtattttcaacagagacgtctgcgagtactacgacccagactacctgaacaccaacgacaagaagaacatcttcctgcagaccatgatcaagctgttcaacagaatcaagtccaagccattgggtgagaagctgctggagatgatcattaacggtatcccatacctgggtgacagaagagtcccactggaggagttcaacaccaacatcgcctccgtcaccgtcaacaagctgatctccaacccgggtgaggtcgagcgtaagaagggcatcttcgccaacctgatcatcttcggcccaggtccagtcttgaacgagaacgagactattgacattggcattcaaaaccacttcgcctccagagagggtttcggcggtatcatgcaaatgaagttctgtccagagtacgtctccgttttcaacaacgtccaagagaacaagggtgcctccatcttcaacagaagaggctacttctccgacccagccttgatcttgatgcacgagttgatccacgtcttgcacggtttgtacggtatcaaggtcgacgacttgccaattgtcccaaacgagaagaagttcttcatgcagtccaccgacgccatccaggccgaggagctgtacaccttcggtggtcaggacccatccatcattaccccatccaccgacaagtccatctacgacaaggtcttgcagaacttcagaggtatcgtcgatagactgaacaaggtcttggtctgcatctccgacccaaacatcaacatcaacatttacaagaacaagttcaaggacaagtacaagttcgtcgaggactccgagggtaagtactccatcgacgtcgagtccttcgacaagctgtacaagtccctgatgttcggtttcaccgagaccaacatcgccgagaactacaagatcaagaccagagcctcctacttctccgactccctgccaccagtcaagatcaagaacttgttggacaacgaaatctacactattgaggagggtttcaacatttccgacaaggacatggagaaggagtacagaggtcaaaacaaggctattaacaagcaagcttacgaggagatttctaaggagcacttggctgtttacaagattcaaatgtgtaagtctgttaagtaatag

SEQ ID NO: 23 - protein, 441 aa, Artificial Sequence; identical to SEQ ID NO: 7
  Recombinant protein encoded by SEQ ID NO22

SEQ ID NO: 24 - DNA, 2559 bp, Artificial Sequence
  Synthetic polynucleotide sequence for the light chain with Hn segment of of C. botulinum Type B, optimized for expression in E. coli.
atgccagttaccatcaacaacttcaactacaacgacccaatcgacaacaacaacatcattatgatggagccaccattcgctagaggtaccggtagatactacaaggctttcaagatcaccgacagaatttggattattccagagagatacactttcggttacaagccagaggacttcaacaagtcttctggtattttcaacagagacgtctgcgagtactacgacccagactacctgaacaccaacgacaagaagaacatcttcctgcagaccatgatcaagctgttcaacagaatcaagtccaagccattgggtgagaagctgctggagatgatcattaacggtatcccatacctgggtgacagaagagtcccactggaggagttcaacaccaacatcgcctccgtcaccgtcaacaagctgatctccaacccgggtgaggtcgagcgtaagaagggcatcttcgccaacctgatcatcttcggcccaggtccagtcttgaacgagaacgagactattgacattggcattcaaaaccacttcgcctccagagagggtttcggcggtatcatgcaaatgaagttctgtccagagtacgtctccgttttcaacaacgtccaagagaacaagggtgcctccatcttcaacagaagaggctacttctccgacccagccttgatcttgatgcacgagttgatccacgtcttgcacggtttgtacggtatcaaggtcgacgacttgccaattgtcccaaacgagaagaagttcttcatgcagtccaccgacgccatccaggccgaggagctgtacaccttcggtggtcaggacccatccatcattaccccatccaccgacaagtccatctacgacaaggtcttgcagaacttcagaggtatcgtcgatagactgaacaaggtcttggtctgcatctccgacccaaacatcaacatcaacatttacaagaacaagttcaaggacaagtacaagttcgtcgaggactccgagggtaagtactccatcgacgtcgagtccttcgacaagctgtacaagtccctgatgttcggtttcaccgagaccaacatcgccgagaactacaagatcaagaccagagcctcctacttctccgactccctgccaccagtcaagatcaagaacttgttggacaacgaaatctacactattgaggagggtttcaacatttccgacaaggacatggagaaggagtacagaggtcaaaacaaggctattaacaagcaagcttacgaggagatttctaaggagcacttggctgtttacaagattcaaatgtgtaagtctgttaaggctccaggaatctgtatcgacgtcgacaacgaggacttgttcttcatcgctgacaagaactccttctccgacgacttgtccaagaacgagagaatcgagtacaacacccagtccaactacatcgagaacgacttcccaatcaacgagttgatcttggacaccgacttgatctccaagatcgagttgccatccgagaacaccgagtccttgactgacttcaacgtcgacgtcccagtctacgagaagcaaccagctatcaagaagattttcaccgacgagaacaccatcttccaatacctgtactctcagaccttccctttggacatcagagacatctccttgacctcttccttcgacgacgccctgctgttctccaacaaggtctactccttcttctccatggactacatcaagactgctaacaaggtcgtcgaggccggtttgttcgctggttgggtcaagcagatcgtcaacgatttcgtcatcgaggctaacaagtccaacaccatggacaagattgccgacatctccttgattgtcccatacatcggtttggccttgaacgtcggtaacgagaccgccaagggtaacttcgagaacgctttcgagatcgctggtgcctccatcttgttggagttcatcccagagttgttgatcccagtcgtcggtgccttcttgttggagtcctacatcgacaacaagaacaagatcatcaagaccatcgacaacgctttgaccaagagaaacgagaagtggtccgacatgtacggtttgatcgtcgcccaatggttgtccaccgtcaacacccaattctacaccatcaaggagggtatgtacaaggccttgaactaccaggcccaagctttggaggagatcatcaagtacagatacaacatctactccgagaaggagaagtccaacattaacatcgacttcaacgacatcaactccaagctgaacgagggtattaaccaggccatcgacaacatcaacaacttcatcaacggttgttccgtctcctacttgatgaagaagatgattccattggccgtcgagaagttgttggacttcgacaacaccctgaagaagaacttgttgaactacatcgacgagaacaagttgtacttgatcggttccgctgagtacgagaagtccaaggtcaacaagtacttgaagaccatcatgccattcgacttgtccatctacaccaacgacaccatcttgatcgagatgttc

SEQ ID NO: 25 - protein, 852 aa, Artificial Sequence
  Recombinant protein encoded by SEQ ID NO24
PVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMF

SEQ ID NO: 26 - DNA, 1311 bp, Artificial Sequence
  Synthetic polynucleotide sequence for the light chain of of C. botulinum Type C, optimized for expression in E. coli.
atgccaatcaccatcaacaacttcaactactcagaccctgtcgacaacaagaacattctgtacctggacactcacctgaacaccctagctaacgagcctgagaaggcctttcggatcaccggaaacatctgggtcatccctgatcgtttctcccgtaactccaaccccaacctgaacaagcctcctcgggtcaccagccctaagagtggttactacgaccctaactacctgagtaccgactctgacaaggacaccttcctgaaggagatcatcaagctgttcaagcgtatcaactcccgtgagatcggagaggagctcatctacagactttcgaccgatatccccttccctggtaacaacaatactccaatcaacaccttcgacttcgacgtcgacttcaactccgtcgacgtcaagactcggcagggtaacaactgggttaagactggtagcatcaacccttccgtcatcatcactggacctcgtgagaacatcatcgacccagagacttccacgttcaagctgactaacaacaccttcgcggctcaagaaggattcggtgctctgtcaatcatctccatctcacctcgtttcatgctgacctactcgaacgcaaccaacgacgtcggagagggtaggttctctaagtctgagttctgcatggacccaatcctgatcctgatgcatgagctgaaccatgcaatgcacaacctgtacggaatcgctatcccaaacgaccagaccatctcctccgtgacctccaacatcttctactcccagtacaacgtgaagctggagtacgcagagatctacgctttcggaggtccaactatcgaccttatccctaagtccgctaggaagtacttcgaggagaaggctttggattactacagatccatcgctaagagactgaacagtatcaccaccgcaaacccttccagcttcaacaagtacatcggtgagtacaagcagaagctgatcagaaagtaccgtttcgtcgtcgagtcttcaggtgaggtcacagtaaaccgtaacaagttcgtcgagctgtacaacgagcttacccagatcttcacagagttcaactacgctaagatctacaacgtccagaacaggaagatctacctgtccaacgtgtacactccggtgacggcgaacatcctggacgacaacgtctacgacatccagaacggattcaacatccctaagtccaacctgaacgtactattcatgggtcaaaacctgtctcgaaacccagcactgcgtaaggtcaaccctgagaacatgctgtacctgttcaccaagttctgc

SEQ ID NO: 27 - protein, 436 aa, Artificial Sequence
  Recombinant protein encoded by SEQ ID NO26
PITINNFNYSDPVDNKNILYLDTHLNTLANEPEKAFRITGNIWVIPDRFSRNSNPNLNKPPRVTSPKSGYYDPNYLSTDSDKDTFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFC

SEQ ID NO: 28 - DNA, 2436 bp, Artificial Sequence
  Synthetic polynucleotide sequence for the light chain with Hn segment of of C. botulinum Type C, optimized for expression in E. coli.
atgccaatcaccatcaacaacttcaactactcagaccctgtcgacaacaagaacattctgtacctggacactcacctgaacaccctagctaacgagcctgagaaggcctttcggatcaccggaaacatctgggtcatccctgatcgtttctcccgtaactccaaccccaacctgaacaagcctcctcgggtcaccagccctaagagtggttactacgaccctaactacctgagtaccgactctgacaaggacaccttcctgaaggagatcatcaagctgttcaagcgtatcaactcccgtgagatcggagaggagctcatctacagactttcgaccgatatccccttccctggtaacaacaatactccaatcaacaccttcgacttcgacgtcgacttcaactccgtcgacgtcaagactcggcagggtaacaactgggttaagactggtagcatcaacccttccgtcatcatcactggacctcgtgagaacatcatcgacccagagacttccacgttcaagctgactaacaacaccttcgcggctcaagaaggattcggtgctctgtcaatcatctccatctcacctcgtttcatgctgacctactcgaacgcaaccaacgacgtcggagagggtaggttctctaagtctgagttctgcatggacccaatcctgatcctgatgcatgagctgaaccatgcaatgcacaacctgtacggaatcgctatcccaaacgaccagaccatctcctccgtgacctccaacatcttctactcccagtacaacgtgaagctggagtacgcagagatctacgctttcggaggtccaactatcgaccttatccctaagtccgctaggaagtacttcgaggagaaggctttggattactacagatccatcgctaagagactgaacagtatcaccaccgcaaacccttccagcttcaacaagtacatcggtgagtacaagcagaagctgatcagaaagtaccgtttcgtcgtcgagtcttcaggtgaggtcacagtaaaccgtaacaagttcgtcgagctgtacaacgagcttacccagatcttcacagagttcaactacgctaagatctacaacgtccagaacaggaagatctacctgtccaacgtgtacactccggtgacggcgaacatcctggacgacaacgtctacgacatccagaacggattcaacatccctaagtccaacctgaacgtactattcatgggtcaaaacctgtctcgaaacccagcactgcgtaaggtcaaccctgagaacatgctgtacctgttcaccaagttctgctccctgtacaacaagacccttgactgtagagagctgctggtgaagaacactgacctgccattcatcggtgacatcagtgacgtgaagactgacatcttcctgcgtaaggacatcaacgaggagactgaggtgatctactacccagacaacgtgtcagtagaccaagtgatcctcagtaagaacacctccgagcatggacaactagacctgctctaccctagtatcgacagtgagagtgagatcctgccaggggagaatcaagtcttctacgacaaccgtacccagaacgtggactacctgaactcctactactacctagagtctcagaagctgagtgacaacgtggaggacttcactttcacgcgttcaatcgaggaggctctggacaacagtgcaaaggtgtacacttacttccctaccctggctaacaaggtgaatgccggtgtgcaaggtggtctgttcctgatgtgggcaaacgacgtggttgaggacttcactaccaacatcctgcgtaaggacacactggacaagatctcagatgtgtcagctatcatcccctacatcggacccgcactgaacatctccaactctgtgcgtcgtggaaacttcactgaggcattcgcagtcactggtgtcaccatcctgctggaggcattccctgagttcacaatccctgctctgggtgcattcgtgatctacagtaaggtccaggagcgaaacgagatcatcaagaccatcgacaactgtctggagcagaggatcaagagatggaaggactcctacgagtggatgatgggaacgtggttgtccaggatcatcacccagttcaacaacatctcctaccagatgtacgactccctgaactaccaggcaggtgcaatcaaggctaagatcgacctggagtacaagaagtactccggaagcgacaaggagaacatcaagagccaggttgagaacctgaagaacagtctggacgtcaagatctcggaggcaatgaacaacatcaacaagttcatccgagagtgctccgtcacctacctgttcaagaacatgctgcctaaggtcatcgacgagctgaacgagttcgaccgaaacaccaaggcaaagctgatcaacctgatcgac

SEQ ID NO: 29 - protein, 811 aa, Artificial Sequence
  Recombinant protein encoded by SEQ ID NO28
PITINNFNYSDPVDNKNILYLDTHLNTLANEPEKAFRITGNIWVIPDRFSRNSNPNLNKPPRVTSPKSGYYDPNYLSTDSDKDTFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLID

SEQ ID NO: 30 - DNA, 1323 bp, Artificial Sequence; identical to SEQ ID NO: 10
  Synthetic polynucleotide sequence for the light chain of of C. botulinum Type D, optimized for expression in E. coli.

SEQ ID NO: 31 - protein, 440 aa, Artificial Sequence
  Recombinant protein encoded by SEQ ID NO30
TWPVKDFNYSDPVNDNDILYLRIPQNKLITTPVKAFMITQNTWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTK

SEQ ID NO: 32 - DNA, 2475 bp, Artificial Sequence
  Synthetic polynucleotide sequence for the light chain with Hn segment of of C. botulinum Type D, optimized for expression in E. coli.
atgacctggccagtcaaggacttcaactactccgacccagtcaacgacaacgacatcttgtacttgagaatcccacaaaacaagttgatcaccaccccagtcaaggctttcatgatcacccagaacacctgggttatcccagagagattctcctccgacaccaacccatccctgtccaagccaccaagaccaacctccaagtaccagtcttactacgacccatcttacttgtctaccgacgagcaaaaggacaccttcttgaagggtattatcaagctgttcaagagaatcaacgagagagacatcggtaagaagttgatcaactacttggtcgttggttccccattcatgggtgactcctctaccccagaggacaccttcgacttcaccagacacaccaccaacattgccgtcgagaagttcgagaacggttcctggaaggtcaccaacatcatcaccccatctgttttgatcttcggtccattgccaaacatcttggactacaccgcctccctgaccttgcaaggtcagcaatccaacccatccttcgagggtttcggtaccctgtctattttgaaggtcgctccagagttcttgttgaccttctccgacgtcacctccaaccaatcctccgccgtcttgggtaagtccatcttctgtatggacccagtcatcgctttgatgcacgagttgacccactccctgcaccagttgtacggtattaacatcccatctgacaagagaatcagaccacaggtctctgagggtttcttctcccaagacggtccaaacgttcagttcgaggagttgtacaccttcggtggtttggacgtcgagattatccaaattgagagatcccaattgagagagaaggctttgggtcactacaaggacatcgccaagagactgaacaacatcaacaagaccattccatcttcctggatctccaacattgacaagtacaagaagattttctccgagaagtacaacttcgacaaggacaacaccggtaacttcgtcgttaacatcgacaagttcaactctttgtactccgacttgaccaacgttatgtctgaggttgtctactcctcccaatacaacgtcaagaacagaacccactacttctccagacactacttgccagttttcgctaacatcttggacgacaacatttacaccatcagagacggtttcaacttgaccaacaagggtttcaacatcgagaactccggtcaaaacatcgagagaaacccagccctgcaaaagctgtcctccgagtctgtcgtcgacttgttcaccaaggtctgtttgagattgaccaagaactcccgtgacgactccacctgcatcaaggtcaagaacaacagactgccatacgttgccgacaaggactccatctcccaggagatcttcgagaacaagatcatcaccgacgagaccaacgttcaaaactactccgacaagttctctttggacgagtccatcctggacggtcaggtcccaatcaacccagagatcgtcgacccactgttgccaaacgtcaacatggagccattgaacttgccaggtgaggagatcgtcttctacgacgacatcaccaagtacgtcgactacttgaactcctactactacttggagtctcaaaagttgtctaacaacgtcgagaacatcaccttgaccacctccgtcgaggaggccttgggttactctaacaagatctacaccttcctgccatccttggctgagaaggttaacaagggtgttcaagctggtttgttcctgaactgggccaacgaggtcgtcgaggacttcaccaccaacatcatgaagaaggacaccctggacaagatctccgacgtctccgtcatcatcccatacatcggtccagccttgaacatcggtaactccgccctgagaggtaacttcaaccaggccttcgccaccgccggtgtcgccttcctgctggagggtttcccagagttcaccatcccagccctgggtgtcttcaccttctactcctccatccaggagagagagaagatcatcaagaccatcgagaactgcttggagcagagagtcaagagatggaaggactcctaccagtggatggtttccaactggctgtccagaatcaccacccaattcaaccacatcaactaccagatgtacgactccctgtcctaccaggccgacgccatcaaggccaagatcgacctggagtacaagaagtactccggttccgacaaggagaacatcaagtcccaggtcgagaacctgaagaactccttggacgtcaagatctccgaggccatgaacaacatcaacaagttcatccgtgagtgttccgtcacctacctgttcaagaacatgctgccaaaggtcatcgacgagctgaacaagttcgacctgagaaccaagaccgagctgatcaacctgatcgactcccacaacatcatcctggttggtgaggttgac

SEQ ID NO: 33 - protein, 824 aa, Artificial Sequence
  Recombinant protein encoded by SEQ ID NO32
TWPVKDFNYSDPVNDNDILYLRIPQNKLITTPVKAFMITQNTWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVD

SEQ ID NO: 34 - DNA, 1283 bp, Artificial Sequence
  Synthetic polynucleotide sequence for the light chain of of C. botulinum Type E, optimized for expression in E. coli.
catatgccgaaaatcaactcgttcaactacaacgacccggtgaatgaccgcacaatcctgtacattaagccgggcggttgccaggagttctacaagagctttaacattatgaagaacatctggatcatccctgaacgcaatgtgatcgggacaacgccacaagatttccaccctccgacttcgctcaaaaacggggactcctcctactacgacccaaattacttgcaaagcgatgaggagaaagatcggttcctgaagattgtgacaaagatcttcaaccgtattaacaacaatctctcggggggcatcctcctggaggaattatccaaggcgaacccttacctgggcaacgacaacactccagacaaccagttccacattggcgacgcctccgcggtggagatcaagttctcgaatggcagtcaggacatccttctccctaatgtcattattatgggcgccgagccggacctttttgaaaccaattccagcaacatctcgctgcgcaacaactacatgccgagcaatcacggctttgggtcgatcgcgatcgtgactttctcgccggagtactcctttcgcttcaacgacaactccatgaacgagttcattcaggacccggcgctcaccctcatgcacgagctgatccactcgttacatggcttgtacggcgcgaaggggatcacgaccaagtataccattacgcagaaacagaacccacttatcacgaacatccgtgggacgaacatcgaggagttcctcacgttcggggggaccgacctgaacattatcaccagcgcccagtccaacgacatttacacgaacctgctggcagattacaaaaaaattgcctccaagctctccaaggtccaggtatcgaacccgttgctcaatccttacaaggacgtcttcgaggctaagtatgggctggataaggatgcctcaggaatctactctgtgaacatcaacaaattcaacgacatcttcaagaagctgtacagcttcaccgagtttgacctcgccaccaagttccaggtcaaatgtcggcaaacgtacattggccagtataaatattttaagctgtcgaatcttctcaacgactctatctataacatctccgaggggtacaatattaacaacttaaaagtcaacttccgagggcagaacgcaaatctcaacccacggattattactcctattacaggccgcgggctcgtcaagaagatcatccgattttgcaaaaacattgtcagcgttaaaggcatccgtaagtaataggatcc

SEQ ID NO: 35 - protein, 427 aa, Artificial Sequence
  Recombinant protein encoded by SEQ ID NO34
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKXXDX

SEQ ID NO: 36 - DNA, 2415 bp, Artificial Sequence
  Synthetic polynucleotide gene sequence for the light chain with Hn segment of of C. botulinum Type E, optimized for expression in E. coli.
catatgccgaaaatcaactcgttcaactacaacgacccggtgaatgaccgcacaatcctgtacattaagccgggcggttgccaggagttctacaagagctttaacattatgaagaacatctggatcatccctgaacgcaatgtgatcgggacaacgccacaagatttccaccctccgacttcgctcaaaaacggggactcctcctactacgacccaaattacttgcaaagcgatgaggagaaagatcggttcctgaagattgtgacaaagatcttcaaccgtattaacaacaatctctcggggggcatcctcctggaggaattatccaaggcgaacccttacctgggcaacgacaacactccagacaaccagttccacattggcgacgcctccgcggtggagatcaagttctcgaatggcagtcaggacatccttctccctaatgtcattattatgggcgccgagccggacctttttgaaaccaattccagcaacatctcgctgcgcaacaactacatgccgagcaatcacggctttgggtcgatcgcgatcgtgactttctcgccggagtactcctttcgcttcaacgacaactccatgaacgagttcattcaggacccggcgctcaccctcatgcacgagctgatccactcgttacatggcttgtacggcgcgaaggggatcacgaccaagtataccattacgcagaaacagaacccacttatcacgaacatccgtgggacgaacatcgaggagttcctcacgttcggggggaccgacctgaacattatcaccagcgcccagtccaacgacatttacacgaacctgctggcagattacaaaaaaattgcctccaagctctccaaggtccaggtatcgaacccgttgctcaatccttacaaggacgtcttcgaggctaagtatgggctggataaggatgcctcaggaatctactctgtgaacatcaacaaattcaacgacatcttcaagaagctgtacagcttcaccgagtttgacctcgccaccaagttccaggtcaaatgtcggcaaacgtacattggccagtataaatattttaagctgtcgaatcttctcaacgactctatctataacatctccgaggggtacaatattaacaacttaaaagtcaacttccgagggcagaacgcaaatctcaacccacggattattactcctattacaggccgcgggctcgtcaagaagatcatccgattttgcaaaaacattgtcagcgttaaaggcatccgtaagtccatctgcatcgagatcaacaacggtgagctgttcttcgtggcttccgagaacagttacaacgatgacaacatcaacactcctaaggagattgacgacaccgtcacttctaacaacaactacgaaaacgacctggaccaggtcatcctaaacttcaactccgagtccgcccctggtctgtccgacgagaagctgaacctgaccatccagaacgacgcttacatcccaaagtacgactccaacggtacatccgatatcgagcagcatgacgttaacgagcttaacgtcttcttctacttagacgctcagaaggtgcccgagggtgagaacaacgtcaatctcacctcttcaattgacacagccttgttggagcagcctaagatctacaccttcttctcctccgagttcatcaacaacgtcaacaagcctgtgcaggccgcattgttcgtaagctggattcagcaggtgttagtagacttcactactgaggctaaccagaagtccactgttgacaagatcgctgacatctccatcgtcgtcccatacatcggtctggctctgaacatcggcaacgaggcacagaagggcaacttcaaggatgcccttgagttgttgggtgccggtattttgttggagttcgaacccgagctgctgatccctaccatcctggtcttcacgatcaagtccttcctgggttcctccgacaacaagaacaaggtcattaaggccatcaacaacgccctgaaggagcgtgacgagaagtggaaggaagtctattccttcatcgtctcgaactggatgaccaagatcaacacccagttcaacaagcgaaaggagcagatgtaccaggctctgcagaaccaggtcaacgccatcaagaccatcatcgagtccaagtacaactcctacaccctggaggagaagaacgagcttaccaacaagtacgatatcaagcagatcgagaacgagctgaaccagaaggtctccatcgccatgaacaacatcgacaggttcctgaccgagtcctccatctcctacctgatgaagctcatcaacgaggtcaagatcaacaagctgcgagagtacgacgagaatgtcaagacgtacctgctgaactacatcatccagcacggatccatcctg

SEQ ID NO: 37 - protein, 804 aa, Artificial Sequence
  Recombinant protein encoded by SEQ ID NO36
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSIL

SEQ ID NO: 38 - DNA, 1334 bp, Artificial Sequence
  Synthetic polynucleotide sequence for the light chain of of C. botulinum Type F, optimized for expression in E. coli.
catatgccggttgtcatcaattcttttaactacaacgacccggtgaacgacgacacgattctgtacatgcaaatcccttacgaggagaagtctaaaaagtattataaggcgttcgagatcatgcgcaacgtgtggatcatcccggaacgcaacactattgggacagacccgtcggacttcgatccgcctgcgtcgcttgaaaacggctcatcagcatactatgacccaaattatttgactacggacgcggaaaaggaccgttatctcaagaccacaatcaagctcttcaagcgtattaactccaacccggcgggcgaggtattgcttcaggagatttcctacgccaagccttacctcggcaatgagcatactcctatcaacgagttccaccctgtgacccgaaccacgtctgtaaacattaagagttcgacgaatgtaaagtcgtcaattattctcaacctcttggtccttggcgcggggccggacatcttcgagaactcttcctacccggttcgcaagctcatggacagtgggggggtctatgacccgagcaacgacgggttcggttccatcaatatcgtgaccttctcacctgagtacgagtatacatttaacgacatcagcggcggctacaacagtagcaccgagtcctttatcgccgacccggccatcagcctcgctcacgagctcatccacgccctgcacgggctgtacggggcccggggcgttacatataaggagaccatcaaagtgaagcaggcgccactcatgattgccgaaaagccaatccgattggaggagttcctgacattcgggggccaggacctgaatattatcactagtgcaatgaaggagaagatttataacaacctgctcgcgaactatgagaagatcgccactcgcttatcccgggtgaactccgccccaccggagtatgacattaacgagtataaagactacttccagtggaagtatggactggataaaaacgcggacgggtcttacaccgtgaacgagaacaaattcaacgagatctacaagaagctctacagcttcacggagatcgacctcgcgaacaagttcaaggtgaagtgccggaacacgtatttcatcaagtacggcttcttaaaggtgccaaacctgttagacgacgacatttataccgtatcggagggcttcaatattggtaatctggccgtgaacaatcgcggccagaatattaaacttaacccgaaaattatcgactcgatcccagacaaggggttagttgagaagatcgtcaagttctgcaagtcggtcatccctcgcaaggggacgaagaattaataggatcc

SEQ ID NO: 39 - protein, 443 aa, Artificial Sequence
  Recombinant protein encoded by SEQ ID NO38
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKNXXD

SEQ ID NO: 40 - DNA, 2577 bp, Artificial Sequence
  Synthetic polynucleotide sequence for the light chain with Hn segment of of C. botulinum Type F, optimized for expression in E. coli.
catatgccggttgtcatcaattcttttaactacaacgacccggtgaacgacgacacgattctgtacatgcaaatcccttacgaggagaagtctaaaaagtattataaggcgttcgagatcatgcgcaacgtgtggatcatcccggaacgcaacactattgggacagacccgtcggacttcgatccgcctgcgtcgcttgaaaacggctcatcagcatactatgacccaaattatttgactacggacgcggaaaaggaccgttatctcaagaccacaatcaagctcttcaagcgtattaactccaacccggcgggcgaggtattgcttcaggagatttcctacgccaagccttacctcggcaatgagcatactcctatcaacgagttccaccctgtgacccgaaccacgtctgtaaacattaagagttcgacgaatgtaaagtcgtcaattattctcaacctcttggtccttggcgcggggccggacatcttcgagaactcttcctacccggttcgcaagctcatggacagtgggggggtctatgacccgagcaacgacgggttcggttccatcaatatcgtgaccttctcacctgagtacgagtatacatttaacgacatcagcggcggctacaacagtagcaccgagtcctttatcgccgacccggccatcagcctcgctcacgagctcatccacgccctgcacgggctgtacggggcccggggcgttacatataaggagaccatcaaagtgaagcaggcgccactcatgattgccgaaaagccaatccgattggaggagttcctgacattcgggggccaggacctgaatattatcactagtgcaatgaaggagaagatttataacaacctgctcgcgaactatgagaagatcgccactcgcttatcccgggtgaactccgccccaccggagtatgacattaacgagtataaagactacttccagtggaagtatggactggataaaaacgcggacgggtcttacaccgtgaacgagaacaaattcaacgagatctacaagaagctctacagcttcacggagatcgacctcgcgaacaagttcaaggtgaagtgccggaacacgtatttcatcaagtacggcttcttaaaggtgccaaacctgttagacgacgacatttataccgtatcggagggcttcaatattggtaatctggccgtgaacaatcgcggccagaatattaaacttaacccgaaaattatcgactcgatcccagacaaggggttagttgagaagatcgtcaagttctgcaagtcggtcatccctcgcaaggggacgaagaattgcaagtccgtcatcccacgtaagggtaccaaggccccaccacgtctgtgtattagagtcaacaactcagaattattctttgtcgcttccgagtcaagctacaacgagaacgatattaacacacctaaagagattgacgatactaccaacctaaacaacaactaccggaacaacttggatgaggttattttggattacaactcacagaccatccctcaaatttccaaccgtaccttaaacactcttgtccaagacaactcctacgttccaagatacgattctaacggtacctcagagatcgaggagtatgatgttgttgactttaacgtctttttctatttgcatgcccagaaggtgccagaaggtgaaaccaacatctcattgacttcttccattgataccgccttgttggaagagtccaaggatatcttcttttcttcggagtttatcgatactatcaacaagcctgtcaacgccgctctgttcattgattggattagcaaggtcatcagagattttaccactgaagctactcaaaagtccactgttgataagattgctgacatctctttgattgtcccctatgtcggtcttgctttgaacatcattattgaggcagaaaagggtaactttgaggaggcttttgaattgttgggagttggtattttgttggagtttgttccagaacttaccattcctgtcattttagtttttacgatcaagtcctacatcgattcatacgagaacaagaataaagcaattaaagctattaacaactccttgatcgaaagagaggctaagtggaaggaaatctactcatggattgtatcaaactggcttactagaattaacactcaatttaacaagagaaaggagcaaatgtaccaggctctgcaaaaccaagtcgatgctatcaagactgcaattgaatacaagtacaacaactatacttccgatgagaagaacagacttgaatctgaatacaatatcaacaacattgaagaagagttgaacaagaaagtttctttggctatgaagaatatcgaaagatttatgaccgaatcctctatctcttacttgatgaagttgatcaatgaggccaaggttggtaagttgaagaagtacgataaccacgttaagagcgatctgctgaactacattctcgaccacagatcaatcctgggagagcagacaaacgagctgagtgatttggttacttccactttgaactcctccattccatttgagctttct

SEQ ID NO: 41 - protein, 858 aa, Artificial Sequence
  Recombinant protein encoded by SEQ ID NO40
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKNCKSVIPRKGTKAPPRLCIRVNNSELFFVASESSYNENDINTPKEIDDTTNLNNNYRNNLDEVILDYNSQTIPQISNRTLNTLVQDNSYVPRYDSNGTSEIEEYDVVDFNVFFYLHAQKVPEGETNISLTSSIDTALLEESKDIFFSSEFIDTINKPVNAALFIDWISKVIRDFTTEATQKSTVDKIADISLIVPYVGLALNIIIEAEKGNFEEAFELLGVGILLEFVPELTIPVILVFTIKSYIDSYENKNKAIKAINNSLIEREAKWKEIYSWIVSNWLTRINTQFNKRKEQMYQALQNQVDAIKTAIEYKYNNYTSDEKNRLESEYNINNIEEELNKKVSLAMKNIERFMTESSISYLMKLINEAKVGKLKKYDNHVKSDLLNYILDHRSILGEQTNELSDLVTSTLNSSIPFELS

SEQ ID NO: 42 - DNA, 1337 bp, Artificial Sequence
  Synthetic polynucleotide sequence for the light chain of of C. botulinum Type G, optimized for expression in E. coli.
catatgccggtcaatattaagaacttcaattacaacgacccgatcaataatgacgatatcattatgatggagcctttcaacgacccaggtccaggcacgtattacaaggcttttcggatcatcgaccgcatttggatcgtcccggagcgcttcacgtacggcttccaacctgaccagttcaatgcaagcacaggggttttcagcaaggacgtctacgagtactatgacccaacttacctgaagactgacgcggagaaggacaaattcctgaagacgatgatcaagttgttcaaccgcattaactccaagccgtccggccagcgactgcttgatatgattgtggacgccatcccttacctcggaaacgcctctacgccaccggacaagttcgcggcaaacgttgcaaacgtgtccatcaacaagaaaattattcagccgggggccgaggaccagattaagggacttatgactaatctgatcatcttcgggccggggcctgtactctcggacaacttcacggacagcatgattatgaacggccattcaccgatctcagaaggattcggggcacgtatgatgatccggttctgcccgagttgcctcaacgtcttcaacaacgtccaggaaaataaggatacatcgatcttctcccgccgtgcctacttcgcggacccagcgttaacccttatgcacgagttaatccacgtattgcacggcctctacggcattaagatctcgaacttacctattaccccaaacacgaaagagttcttcatgcaacacagcgacccggttcaggccgaggaattatacaccttcggcgggcacgacccaagtgttatctcaccgtctaccgatatgaatatctacaacaaggccctgcaaaacttccaggacatcgcaaaccggcttaacattgtctcatcggcacaggggtctggtatcgacatctccctgtataagcagatctacaagaataagtacgacttcgtagaagacccgaacggcaagtactcggtggacaaggacaagtttgacaaactctacaaagctctcatgttcggtttcacagagacaaatcttgccggagagtacgggatcaagacgcggtactcgtatttttccgagtacctgccgcctattaagacggagaagttgctcgataacaccatttacactcagaatgaggggttcaacatcgcctctaagaatctcaagaccgagttcaatggtcagaacaaggcggtgaacaaagaggcgtatgaggagattagtctggaacacttggtgatctaccgaattgcgatgtgtaagcctgtgatgtactaataggatcc

SEQ ID NO: 43 - protein, 444 aa, Artificial Sequence
  Recombinant protein encoded by SEQ ID NO42
MPVNIKNFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYXXD

SEQ ID NO: 44 - DNA, 2547 bp, Artificial Sequence
  Synthetic polynucleotide sequence for the light chain with Hn segment of of C. botulinum Type G, optimized for expression in E. coli.
catatgccggtcaatattaagaacttcaattacaacgacccgatcaataatgacgatatcattatgatggagcctttcaacgacccaggtccaggcacgtattacaaggcttttcggatcatcgaccgcatttggatcgtcccggagcgcttcacgtacggcttccaacctgaccagttcaatgcaagcacaggggttttcagcaaggacgtctacgagtactatgacccaacttacctgaagactgacgcggagaaggacaaattcctgaagacgatgatcaagttgttcaaccgcattaactccaagccgtccggccagcgactgcttgatatgattgtggacgccatcccttacctcggaaacgcctctacgccaccggacaagttcgcggcaaacgttgcaaacgtgtccatcaacaagaaaattattcagccgggggccgaggaccagattaagggacttatgactaatctgatcatcttcgggccggggcctgtactctcggacaacttcacggacagcatgattatgaacggccattcaccgatctcagaaggattcggggcacgtatgatgatccggttctgcccgagttgcctcaacgtcttcaacaacgtccaggaaaataaggatacatcgatcttctcccgccgtgcctacttcgcggacccagcgttaacccttatgcacgagttaatccacgtattgcacggcctctacggcattaagatctcgaacttacctattaccccaaacacgaaagagttcttcatgcaacacagcgacccggttcaggccgaggaattatacaccttcggcgggcacgacccaagtgttatctcaccgtctaccgatatgaatatctacaacaaggccctgcaaaacttccaggacatcgcaaaccggcttaacattgtctcatcggcacaggggtctggtatcgacatctccctgtataagcagatctacaagaataagtacgacttcgtagaagacccgaacggcaagtactcggtggacaaggacaagtttgacaaactctacaaagctctcatgttcggtttcacagagacaaatcttgccggagagtacgggatcaagacgcggtactcgtatttttccgagtacctgccgcctattaagacggagaagttgctcgataacaccatttacactcagaatgaggggttcaacatcgcctctaagaatctcaagaccgagttcaatggtcagaacaaggcggtgaacaaagaggcgtatgaggagattagtctggaacacttggtgatctaccgaattgcgatgtgtaagcctgtgatgtacaagaacaccggtaagtccgagcagtgtatcatcgtcaacaacgaggacttgttcttcatcgccaacaaggactccttctccaaggacttggccaaggctgagaccatcgcctacaacacccagaacaacaccatcgagaacaacttctccatcgaccagctgatcttggacaacgacctgtcctccggtatcgacctgccaaacgagaacaccgagccattcaccaacttcgacgacatcgacatcccagtctacatcaagcagtccgccctgaagaagatcttcgtcgacggtgactccttgttcgagtacctgcacgcccagaccttcccatccaacatcgagaaccagttgaccaactccctgaacgacgctttgagaaacaacaacaaggtctacaccttcttctccactaacttggtcgagaaggccaacactgtcgtcggtgcctccttgttcgtcaactgggtcaagggtgtcatcgacgacttcacctccgagtccacccaaaagtccaccatcgacaaggtctccgacgtctccatcatcatcccatacatcggtccagccctgaacgtcggtaacgagaccgctaaggagaacttcaagaacgccttcgagatcggtggtgccgccatcctgatggagttcatcccagagttgatcgtcccaatcgtcggtttcttcaccttggagtcctacgtcggtaacaagggtcacatcatcatgaccatctccaacgccctgaagaagagagaccagaagtggaccgacatgtacggtttgatcgtctcccagtggttgtccaccgtcaacacccagttctacaccatcaaggagagaatgtacaacgccttgaacaaccagtcccaggccatcgagaagatcatcgaggaccagtacaaccgttactccgaggaggacaagatgaacatcaacatcgacttcaacgacatcgacttcaagctgaaccagtccatcaacctggccatcaacaacatcgacgacttcatcaaccagtgttccatctcctacctgatgaaccgtatgatcccactggccgtcaagaagttgaaggacttcgacgacaacctgaagcgtgacctgctggagtacatcgacaccaacgagttgtacctgctggacgaggtcaacatcttgaagtccaaggtcaacagacacttgaaggactccatcccattcgacttgtccttgtacacc

SEQ ID NO: 45 - protein, 848 aa, Artificial Sequence
  Recombinant protein encoded by SEQ ID NO44
MPVNIKNFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYT

SEQ ID NO: 46 - protein, 7 aa, Artificial Sequence
  Synthetic peptide; competative inhibitor of Zn protease
CRATKML

SEQ ID NO: 47 - protein, 449 aa, Artificial Sequence
  Synthetic botulinum neurotoxin light chain of serotype A based on wild-type Clostridium botulinum sequence
MVQFVNKQFNYKDPVNGVDIAYIKIPNVGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNK

(19) United States
(12) Patent Application Publication
(10) Pub. No.: US 2006/0141572 A1
(43) Pub. Date: Jun. 29, 2006

(54) Recombinant light chains of botulinum neurotoxins and light chain fusion proteins for use in research and clinical therapy

(76) Inventors: Leonard A. Smith, Clarksburg, MD (US); Melody Jensen, Frederick, MD (US)

Correspondence Address: BAKER & BOTTS, 30 ROCKEFELLER PLAZA, 44TH FLOOR, NEW YORK, NY, 10112, US

(21) Appl. No.: 11/293,582

(22) Filed: Dec. 2, 2005

Related U.S. Application Data
(62) Division of application No. 10/011,588, filed on Nov. 6, 2001, now Pat. No. 7,037,680.
Application No. 10/011,588 is a continuation-in-part of application No. 09/910,186, filed on Jul. 20, 2001.
Application No. 09/910,186 is a continuation of application No. 09/611,419, filed on Jul. 6, 2000.
Application No. 09/611,419 is a continuation of application No. 08/123,975, filed on Sep. 21, 1993, now abandoned.
(60) Provisional application No. 60/133,866, filed on May 12, 1999.
(60) Provisional application No. 60/133,868, filed on May 12, 1999.
(60) Provisional application No. 60/133,869, filed on May 12, 1999.
(60) Provisional application No. 60/133,865, filed on May 12, 1999.
(60) Provisional application No. 60/133,873, filed on May 12, 1999.
(60) Provisional application No. 60/133,867, filed on May 12, 1999.

(51) Int. Cl. C12P 21/06; C12N 1/18; C12N 1/21; C07K 14/33

(52) U.S. Cl. 435/69.1; 435/252.33; 435/254.23; 530/350

(57) ABSTRACT

Botulinum neurotoxins, the most potent of all toxins, induce lethal neuromuscular paralysis by inhibiting exocytosis at the neuromuscular junction. The light chains (LC) of these dichain neurotoxins are a new class of zinc-endopeptidases that specifically cleave the synaptosomal proteins, SNAP-25, VAMP, or syntaxin at discrete sites. The present invention relates to the construction, expression, purification, and use of synthetic or recombinant botulinum neutoroxin genes. For example, a synthetic gene for the LC of the botulinum neurotoxin serotype A (BoNT/A) was constructed and overexpressed in Escherichia coli. The gene product was purified from inclusion bodies. The methods of the invention can provide 1.1 g of the LC per liter of culture. The LC product was stable in solution at 4° C. for at least 6 months. This rBoNT/A LC was proteolytically active, specifically cleaving the Glu-Arg bond in a 17-residue synthetic peptide of SNAP-25, the reported cleavage site of BoNT/A. Its calculated catalytic efficiency k_{cat}/Kₘ was higher than that reported for the native BoNT/A dichain. Treating the rBoNT/A LC with mercuric compounds completely abolished its activity, most probably by modifying the cysteine-164 residue located in the vicinity of the active site. About 70% activity of the LC was restored by adding Zn²⁺ to a Zn²⁺-free, apo-LC preparation. The LC was nontoxic to mice and failed to elicit neutralizing epitope(s) when the animals were vaccinated with this protein. In addition, injecting rBoNT/A LC into sea urchin eggs inhibited exocytosis-dependent plasma membrane resealing.

### FIELD OF THE INVENTION

This invention is directed to construction, expression, and purification of synthetic DNA molecules encoding polypeptides comprising botulinum neurotoxin (BoNT) light chains. The invention is also directed to methods of vaccination against botulism using the expressed peptides.

### BACKGROUND OF THE INVENTION

The sporulating, obligate anaerobic, gram-positive bacillus Clostridium produces eight forms of antigenically distinct exotoxins. Tetanus neurotoxin (TeNT) is produced by Clostridium tetani while Clostridium botulinum produces seven different neurotoxins which are differentiated serologically by specific neutralization. The botulinum neurotoxins (BoNT) have been designated as serotypes A, B, C₁, D, E, F, and G. Botulinum neurotoxins (BoNT) are the most toxic substances known and are the causative agents of the disease botulism. BoNT exert their action by inhibiting the release of the neurotransmitter acetylcholine at the neuromuscular junction (Habermann, E., et al., (1986), “Clostridial Neurotoxins: Handling and Action at the Cellular and Molecular Level,” Cur. Top. Microbiol. Immunol., 129:93-179; Schiavo, G., et al., (1992a), “Tetanus and Botulinum-B Neurotoxins Block Neurotransmitter Release by Proteolytic Cleavage of Synaptobrevin,” Nature, 359:832-835; Simpson, L. L., (1986), “Molecular Pharmacology of Botulinum Toxin and Tetanus Toxin,” Annu. Rev. Pharmacol. Toxicol., 26:427-453) which leads to a state of flaccid paralysis. Indeed, only a few molecules of toxin are required to abolish the action of a nerve cell. Polyclonal antibodies derived from a specific neurotoxin can neutralize the toxic effects of that toxin but will not cross-neutralize another toxin serotype. Thus, to protect against all seven toxins, one needs seven vaccines.

Human botulism poisoning is generally caused by type A, B, E or rarely, by type F toxin. Type A and B are highly poisonous proteins which resist digestion by the enzymes of the gastrointestinal tract. Foodborne botulism poisoning is caused by the toxins present in contaminated food, but wound and infant botulism are caused by in vivo growth in closed wounds and the gastrointestinal tract respectively. The toxins primarily act by inhibiting the neurotransmitter acetylcholine at the neuromuscular junction, causing paralysis. Another means for botulism poisoning to occur is the deliberate introduction of the toxin(s) into the environment as might occur in biological warfare or a terrorist attack. When the cause of botulism is produced by toxin rather than by in vivo infection the onset of neurologic symptoms is usually abrupt and occurs within 18 to 36 hours after ingestion. The most common immediate cause of death is respiratory failure due to diaphragmatic paralysis. Home canned foods are the most common sources of toxins. The most frequently implicated toxin is toxin A, which is responsible for more than 50% of morbidity resulting from botulinum toxin.

Botulinum and tetanus neurotoxins are a new class of zinc-endopeptidases that act selectively at discrete sites on three synaptosomal proteins of the neuroexocytotic apparatus. See Montecucco and Schiavo, 1995, and Schiavo, 1995, for review. These neurotoxins are the most potent of all the known toxins. The botulinum neurotoxins (BONT), designed A-G, produced by seven immunologically distinct strains of Clostridium botulinum cause death by flaccid muscle paralysis at the neuromuscular junction. Extreme toxicity of these toxins and their lability in purified preparations have limited any detailed characterizations.

These neurotoxins are expressed as 150-kDa single polypeptides (termed dichains) containing a disulfide bond between the 50-kDa N-terminal light chain (LC) and the 100-kDa C-terminal heavy chain (HC). A post-translational cryptic cleavage generates the two chains connected by a disulfide bond. The LC contains the toxic, zinc-endopeptidase catalytic domain. The 100-kDa HC may be further proteolyzed into a 50-kDa N-terminal membrane-spanning domain (Hₙ) and a 50-kDa C-terminal receptor-binding domain (H_{c}).

With three functional domains, the mechanism of action of these neurotoxins is multiphasic: (1) The H_{c} domain plays a role in binding the toxins to specific receptors located exclusively on the peripheral cholinergic nerve endings (Black and Dolly, 1986). (2) The Hₙ domain is believed to participate in a receptor-mediated endocytotic pore formation in an acidic environment, allowing translocation of the catalytic LC into the cytosol. Reducing the disulfide bond connecting the LC with the H upon exposure to the cytosol or within the acidic endosome (Montal et al., 1992) releases the catalytic LC into the cytosol. (3) The LC then cleaves at specific sites of one of the three different soluable NSF attachment protein receptor (SNARE) proteins, synaptobrevin, syntaxin, or synaptosomal associated protein of 25 kDa (SNAP-25) (Blasi et al., 1993; Schiavo et al., 1993, 1994; Shone et al., 1993; Foran et al., 1996). These proteins are essential for synaptic vesicle fusion in exocytosis. Their proteolysis inhibits exocytosis and blocks acetylcholine secretion, leading ultimately to muscular paralysis. The LC itself is nontoxic because it cannot translocate through the cholinergic nerve ending into the cytosol. However, in digitonin-permeabilized chromaffin cells, the LC inhibits exocytosis (Bittner et al., 1989), and direct microinjection of the LC into the cytosol results in blockage of membrane exocytosis (Bittner et al., 1989; Bi et al., 1995).

The LC of all known clostridial neurotoxins contain the sequence HExxH that is characteristic of zinc-endoproteinases (Thompson et al., 1990). The essential role of zinc on the structure and catalysis of the neurotoxins is established (Fu et al., 1998). A unique feature of the neurotoxins' protease activity is their substrate requirement. Short peptides encompassing only the cleavage sites are not hydrolyzed (Foran et al., 1994; Shone and Roberts, 1994). A specific secondary and/or tertiary structure of the substrate is most probably recognized (Washboume et al., 1997; Lebeda and Olson, 1994; Rossetto et al., 1994) rather than a primary structure alone, as is the case with most other proteases. Most importantly, their identified natural substrates are proteins involved in the fundamental process of exocytosis (Blasi et al., 1993; Schiavo et al., 1993, 1994; Shone et al., 1993; Foran et al., 1996). Light chain also is the target of an intensive effort to design drugs, inhibitors, and vaccines. A detailed understanding of its structure and function is thus very important.

The present invention describes the construction and overexpression of a synthetic gene for the nontoxic LC of BoNT/A in E. coli. The high level of expression obtained enabled purification of gram quantities of LC from 1 L of culture as well as extensive characterization. The preparation of the rBoNT/A LC was highly soluble, stable at 4° C. for at least 6 months, and had the expected enzymatic and functional properties. For the first time, a cysteine residue was tentatively identified in the vicinity of the active site which, when modified by mercuric compounds, led to complete loss of enzymatic activity.

The BoNTs and their LCs are targets of vaccine development, drug design, and mechanism studies because of their potential role in biological warfare, wide therapeutic applications, and potential to facilitate elucidation of the mechanism of membrane exocytosis. In spite of such immense importance, studies of the LC have been limited by its availability. Commercially available LC is prepared by separating it from the dichain toxins under denaturing conditions. These preparations therefore retain some contaminating toxicity of the dichain, have low solubility, and often begin to proteolytically degrade and start losing activity within hours of storage in solution.

The LC of serotype A has been separated and purified from the full-length toxin by QAE-Sephadex chromatography from 2 M urea; however, the preparation suffers from low solubility (Shone and Tranter, 1995). The LC of serotype C was similarly obtained at a level of <5 mg/10 L culture of C. botulinum (Syuto and Kubo, 1981). These preparations almost invariably contain contaminating full-length toxins, and the commercially available preparations precipitate from solution or undergo proteolytic degradation upon hours of storage in solution. More recently the LC of tetanus neurotoxin (Li et al., 1994) and of BoNT/A (Zhou et al., 1995) were expressed in E. coli as maltose-binding proteins and purified in 0.5 mg quantities from 1-L cultures (Zhou et al., 1995). However, the poor expression of the cloned products, probably due to rare codon usage in clostridial DNA (Makoff et al., 1989, Winkler and Wood, 1988), remained a major hurdle in obtaining adequate amount of the protein for structural and functional studies.

Most of the clostridial strains contain specific endogenous proteases which activate the toxins at a protease-sensitive loop located approximately one third of the way into the molecule from the amino-terminal end. Upon reduction and fractionation (electrophoretically or chromatographically), the two chains can be separated; one chain has a Mr of ˜100 kDa and is referred to as the heavy chain while the other has a Mr ˜50 kDa and is termed the light chain.

The mechanism of nerve intoxication is accomplished through the interplay of three key events, each of which is performed by a separate portion of the neurotoxin protein. First, the carboxy half of the heavy chain (fragment C or H_{C} is required for receptor-specific binding to cholinergic nerve cells (Black, J. D., et al., (1986), “Interaction of ¹²⁵I-botulinum Neurotoxins with Nerve Terminals. I. Ultrastructural Autoradiographic Localization and Quantitation of Distinct Membrane Acceptors for Types A and B on Motor Nerves,” J. Cell Biol., 103:521-534; Nishiki, T.-I., et al., (1994), “Identification of Protein Receptor for Clostridium botulinum Type B Neurotoxin in Rat Brain Synaptosomes,” J. Biol. Chem., 269:10498-10503; Shone, C. C., et al., (1985), “Inactivation of Clostridium botulinum Type A Neurotoxin by Trypsin and Purification of Two Tryptic Fragments. Proteolytic Action Near the COOH-terminus of the Heavy Subunit Destroys Toxin-Binding Activity, Eur. J. Biochem., 151:75-82). Evidence suggests that polysialogangliosides (van Heyningen, W. E., (1968), “Tetanus,” Sci. Am., 218:69-77) could act as receptors for the toxins but the data supporting a specific receptor remains equivocal (Middlebrook, J. L., (1989), “Cell Surface Receptors for Protein Toxins,” Botulinum Neurotoxins and Tetanus Toxin, (Simpson, L. L., Ed.) pp. 95-119, Academic Press, New York). After binding, the toxin is internalized into an endosome through receptor-mediated endocyctosis (Shone, C. C., et al., (1987), “A 50-kDa Fragment from the NH₂-terminus of the Heavy Subunit of Clostridium botulinum Type A Neurotoxin Forms Channels in Lipid Vesicles, Euro. J. Biochem., 167:175-180).

The amino terminal half of the heavy chain is believed to participate in the translocation mechanism of the light chain across the endosomal membrane (Simpson, 1986; Poulain, B., et al., (1991), “Heterologous Combinations of Heavy and Light Chains from Botulinum Neurotoxin A and Tetanus Toxin Inhibit Neurotransmitter Release in Aplysia,” J. Biol. Chem., 266:9580-9585; Montal, M. S., et al., (1992), “Identification of an Ion Channel-Forming Motif in the Primary Structure of Tetanus and Botulinum Neurotoxins,” FEBS, 313:12-18). The low pH environment of the endosome may trigger a conformational change in the translocation domain, thus forming a channel for the light chain.

The final event of intoxication involves enzymatic activity of the light chain, a zinc-dependent endoprotease (Schiavo, 1992a; Schiavo, G., et al., (1992b), “Tetanus Toxin is a Zinc Protein and its Inhibition of Neurotransmitter Release and Protease Activity Depend on Zinc,” EMBO J, 11:3577-3583), on key synaptic vesicle proteins (Schiavo, 1992a; Oguma, K., et al., (1995), “Structure and Function of Clostridium botulinum Toxins,” Microbiol. Immunol., 39:161-168; Schiavo, G., et al., (1993), “Identification of the Nerve Terminal Targets of Botulinum Neurotoxin Serotypes A, D, and E,” J. Biol. Chem., 268:23784-23787; Shone, C. C., et al., (1993), “Proteolytic Cleavage of Synthetic Fragments of Vesicle-Associated Membrane Protein, Isoform-2 by Botulinum Type B Neurotoxin,” Eur. J. Biochem., 217:965-971) necessary for neurotransmitter release. The light chains of BoNT serotypes A, C₁, and E cleave SNAP-25 (synaptosomal-associated protein of M25,000), serotypes B, D, F, and G cleave vessicle-associated membrane protein (VAMP)/synaptobrevin (synaptic vesicle-associated membrane protein); and serotype C₁ cleaves syntaxin. Inactivation of SNAP-25, VAMP, or syntaxin by BoNT leads to an inability of the nerve cells to release acetylcholine resulting in neuromuscular paralysis and possible death, if the condition remains untreated.

The majority of research related to botulinum toxin has focused on the development of vaccines. Currently, a pentavalent toxoid vaccine against serotypes A through E (Anderson, J. H., et al., (1981), “Clinical Evaluation of Botulinum Toxoids,” Biomedical Aspects of Botulism, (Lewis, G. E., Ed.), pp. 233-246, Academic Press, New York; Ellis, R. J., (1982), “Immunobiologic Agents and Drugs Available from the Centers for Disease Control. Descriptions, Recommendations, Adverse Reactions and Scrologic Response,” 3rd ed., Centers for Disease Control. Atlanta, Ga.; Fiock, M. A., et al., (1963), “Studies of Immunities to Toxins of Clostridium botulinum. IX. Immunologic Response of Man to Purified Pentavalent ABCDE Botulinum Toxoid,” J. Immunol., 90:697-702; Siegel, L. S., (1988), “Human Immune Response to Botulinum Pentavalent (ABCDE) Toxoid Determined by a Neutralization Test and by an Enzyme-Linked Immunosorbent Assay,” J. Clin. Microbiol., 26:2351-2356), available under Investigational New Drug (IND) status, is used to immunize specific populations of at-risk individuals, i.e., scientists and health care providers who handle BoNT and military personnel who may be subjected to weaponized forms of the toxin. Though serotypes A, B, and E are most associated with botulism outbreaks in humans, type F has also been diagnosed (Midura, T. F., et al., (1972), “Clostridium botulinum Type F: Isolation from Venison Jerky,” Appl. Microbiol., 24:165-167; Green, J., et al., (1983), “Human Botulism (Type F)— A Rare Type,” Am. J. Med. 75:893-895; Sonnabend, W. F., et al., (1987), “Intestinal Toxicoinfection by Clostridium botulinum Type F in an Adult. Case Associated with Guillian-Barre Syndrome,” Lancet, 1:357-361; Hatheway, C. L., (1976), “Toxoid of Clostridium botulinum Type F: Purification and Immunogenicity Studies,” Appl. Environ. Microbiol., 31:234-242). A separate monovalent toxoid vaccine against BoNTF is available under IND status. Hatheway demonstrated that the BoNTF toxoid could protect guinea pigs against a homologous challenge (Wadsworth, J. D. F., et al., (1990), “Botulinum Type F Neurotoxin,” Biochem. J., 268:123-128).

New-generation, recombinant vaccines have also been developed by USAMRIID (e.g. Dertzbaugh M T, Sep. 11, 2001, U.S. Pat. No. 6,287,566; U.S. application Ser. No. 09/910,186 filed Jul. 20, 2001; and U.S. application Ser. No. 09/611,419 filed Jul. 6, 2000) and commercial sources (e.g. Ophidian Pharmaceuticals, Inc. Williams J A, Jul. 6, 1999, U.S. Pat. No. 5,919,665; using clones supplied by USAMRIID).

Most vaccine studies have focused on the botulinum toxin heavy chain, leaving the light chain largely ignored. In 1995, Zhou et al. discovered that a single mutation in the light chain of botulinum neurotoxin serotype A abolished its neurotoxicity and its ability to cleave SNAP-25, one of the natural substrates, when reconstituted with the heavy chain. See Zhou, L. et al., (1995), “Expression and Purification of Botulinum Neurotoxin A: A Single Mutation Abolishes its Cleavage of SNAP-25 and Neurotoxicity after Reconstitution with the Heavy Chain,” Biochem., 34:15175-15181.) This raised the possibility that the mutated light chain might have various research or therapeutic uses. Further research produced a recombinant light chain (Li, L. and Singh, B. R., (1999), “High-Level Expression, Purification, and Characterization of Recombinant Type A Botulinum Neurotoxin Light Chain,” Protein Expression and Purification, 17:339-344) and a construct comprising the minimum essential light chain domain (Kadkhodayan, S., et al., (2000), “Cloning, Expression, and One-Step Purification of the Minimal Essential Domain of the Light Chain of Botulinum Neurotoxin Type A,” Protein Expression and Purification, 19:125-130).

Recombinant production methods alleviate many of the problems associated with the toxoid, such as the need for a dedicated manufacturing facility. Presently, many cGMP facilities are in existence and available that could manufacture a recombinant product. There would be no need to culture large quantities of a hazardous toxin-producing bacterium. Production yields from a genetically engineered product are expected to be high. Recombinant products would be purer, less reactogenic, and more fully characterized. Thus, the cost of a recombinant product would be expected to be much lower than a toxoid because there would be no expenditures required to support a dedicated facility, and the higher production yields would reduce the cost of therapeutic and research products.

However, recombinant methods as described in the publications above do not yield optimal results because botulinum codons are not translated well in other organisms commonly used for production, such as E. coli or yeast. Furthermore, no easily translatable, recombinant form of the non-neurotoxic, mutated light chain presently exists. Recombinant forms of both functional and non-neurotoxic botulinum neurotoxin that may be translated efficiently in either E. coli or yeast are needed for research and therapeutic purposes.

Commercially available BoNT LC is prepared by separation from the di-chain toxins. These preparations, therefore, retain some contaminating toxicity, have low solubility, and undergo proteolytic degradation within hours and days of storage in solution. Many clinical disorders are presently being treated with a botulinum neurotoxin complex that is isolated from the bacterium, Clostridium botulinum. There is no data to demonstrate that the binding proteins play any role in the therapeutic effects of the drug. The binding proteins, however, probably contribute to the immunological response in those patients that become non-responsive to drug treatment. Recombinant products could be manufactured under conditions that are more amenable to product characterization. Chimeras of the drug product could also be produced by domain switching. Chimeras could potentially increase the number of potential useful drug products.

Recently, the BoNT LC of serotype A has been expressed as a maltose-binding protein and purified in 0.5 mg quantities from 1 liter culture (Zhou et al., 1995). The poor expression of the native gene was probably due to the high A+T composition found in the clostridial DNA.

### SUMMARY OF THE INVENTION

The present invention relates to the design and construction of synthetic DNA molecules that encode one of the seven light chains of Clostridium botulinum neurotoxin and are capable of being expressed in heterologous prokaryotic or eukaryotic hosts. The invention is based, in part, on modifying the wild-type BoNT sequence according to the codon usage normally found in genes that are highly expressed in the host organism. By selecting codons rich in G+C content, the synthetic DNA molecules may further be designed to lower the high A+T rich base composition found in clostridial genes.

The invention further relates to methods of expressing and purifying recombinant BoNT light chains. According to the invention, BoNT LC may be expressed in a heterologous host system by itself or as a fusion to another protein or carrier. For example, the BoNT LC may be fused to a synthetic or wild-type BoNT heavy chain or a fragment thereof. BoNT LC of the invention may or may not have catalytic activity as a zinc protease. In some embodiments of the invention, catalytically inactive BoNT LC is fused to a BoNT heavy chain forming a mutant holotoxin. Non-enzymatic, non-toxic mutant holotoxins are capable of being internalized into nerve cells. In addition, mutant holotoxins may be used as transporters to carry other molecules into colinergic nerve cells.

The invention further provides methods and compositions for eliciting an immune response to BoNT LC and BoNT HN. The invention provides preparations of BoNT LC and BoNT HN that are capable of eliciting protective immunity in a mammal.

### BRIEF DESCRIPTION OF THE DRAWINGS

FIG. 1. Nucleotide sequence of rBoNT/A LC and the corresponding amino acid sequence. The codon in italics (i.e., encoding the penultimate Val residue) and at the 5′ end of the gene was introduced to create and maintain the Nco I restriction enzyme site. Codons in italics (i.e., encoding LVPRGS; residues 450-455 of SEQ ID NO:5) at the 3′ end of the gene encode a thrombin protease cleavage site for removing the His tag after purification.

FIG. 2. SDS-PAGE followed by Coomassie stain (A) and Western blot (B) of crude and purified BONT/A LC expressed in E. coli containing the synthetic gene for BONT/A LC in a multicopy plasmid pET24. Total cellular protein (T), soluble supernatant (S), insoluble pellet (P), and purified inclusion bodies (I) were prepared as described in Section 2. Lane 1 shows Novex wide-range molecular-mass markers (0.8-3.0 μg/band). The sarkosyl solubilized inclusion bodies of the LC had the same electrophoretic behavior as (I). About 20 μg of protein was applied per lane. Western blot used affinity-purified rabbit polyclonal antibodies against a 16-residue N-terminal sequence of the BONT/A LC as the primary antibody and a peroxidase-coupled goat anti-rabbit IgG (H+ L) as the secondary antibody. Bands were visualized by a chromogenic substrate.

FIG. 3. UV-visible absorption spectrum of the rBoNT/A LC.

FIG. 4. Long-term stability at 4° C. (A) and thermal stability (B) of the rBoNT/A LC. (A) Aliquots of the LC from one single preparation were assayed at the indicated times; (B) 50 μl aliquots of the LC in buffer G containing 1 mM DTT and 50 μM ZnCl₂ were taken in Eppendorf tubes and heated for 5 min at the indicated temperatures. After cooling on ice for 60 min, the supernatants were assayed for proteolytic activity.

FIG. 5. Proteolysis of the synthetic peptide substrate by the rBoNT/A LC. The peptide (1.1 mM) was incubated for 5 min (A) or 200 min (B) with the rBoNT/A LC. The reaction products were analyzed by reverse-phase HPLC. The first three peaks represent the solvent front (<4 min) and reduced DTT (5.2 min) in the reaction mixture. Sequence of the substrate (SEQ ID NO:2) and the sequences of the products (residues 1 to 11 and residues 12 to 17 of SEQ ID NO:2) are shown in panels A and B, respectively. The numbers above the sequences represent the LC residue numbers corresponding to the sequence of SNAP-25. The product peaks (not labeled in Panel A) were identified by sequence determination by MS-MS.

FIG. 6. Effect of pH on the endopeptidase activity of the rBoNT/A LC. Activities were measured at various pH of 0.1 M buffers: MES (-±-), HEPES (-●-), and tris-HCl (−)-) containing 0.9 mM substrate peptide Maximum activity (100%) was 334 nmol/min/mg LC.

FIG. 7. Inhibition of endopeptidase activity of the rBoNT/A LC by excess Zn²⁺ and protection from inhibition by DTT. The LC was assayed in SO mM HEPES, pH 7.4, containing 0.9 mM substrate peptide in the absence (-±-) and presence of 5 mM DTT (-●-) or 5 mM mercaptoethanol (−)-) containing the indicated concentrations of ZnCl₂. One hundred percent activity (290 nmol/min/mg LC) represents the activity obtained in the absence of any added thiol or Zn²⁺.

FIG. 8. Determination of Kₘ and Vₘₐₓ from the double-reciprocal (Lineweaver-Burke) plot of initial rates of proteolysis versus substrate concentration by the rBoNT/A LC. The reaction mixtures (0.03 ml) contained 0.25 mM ZnCl₂, 0.5 mM DTT, 50 mM HEPES, pH 7.4, and 0.016 mg rBoNT/A LC. The Kₘ and Vₘₐₓ were calculated as 0.9 mM and 1500 nmol/min/mg, respectively.

FIG. 9. Location of the three Cys residues in the BoNT/A LC. Molecular surface of the LC portion of the BoNT/A dichain based on its three-dimensional structure (Lacy and Stevens, 1999) is shown. The three Cys residues (yellow), active-site His and asp residues (red), the Zn²⁺ atom (blue) at the active site, and the ‘pit’ leading to the active site are highlighted. The side chain of Cys-164 lines the surface and forms part of the wall of the ‘pit’ leading to the active site. The ‘pit’ acts as an access route of the substrate.

FIG. 10. Time course of proteolysis of BoNT/A LC as followed by SDS-PAGE (A) and Western blot (B). Aliquots of 25 ml of the LC (0.2 mg/ml) were incubated at 4° C. At intervals (see below), 25:1 of 2×SDS-load buffer was added to an aliquot and boiled. Two SDS gels were run in parallel. One gel was stained by Coomassie (A) and the proteins from the other were transferred to a nitrocellulose membrane for Western blot (B). Lane 1 in panel A shows Novex Mark-12 molecular weight markers and lane 1 in panel B shows the Novex prestained See Blue molecular weight markers. In both panels A and B, lanes 2-7 show 0, 2, 4, 14, 21, and 28 days of incubation, respectively, of LC. Identity of the protein bands between panels A and B is arbitrary, and the same nomenclature is used throughout the paper.

FIG. 11. Enhancement of the proteolysis of BoNT/A LC by ZnCl₂ as followed by SDS-PAGE (A) and Western blot (B). All conditions are same as in FIG. 10, except that 0.25 mM ZnCl₂ was added to the incubation mixture of the LC.

FIG. 12. Protection of BoNT/A LC from proteolysis by the metal chelator TPEN (A) and the competitive peptide inhibitor CRATKML (SEQ ID NO:46) (B), followed as a time course by SDS-PAGE. (A) the LC (0.2 mg/ml) was incubated in small aliquots with 10 mM EDTA (lanes 2-5) or with 5 mM TPEN (lanes 7-10). Lanes 2 and 7, 3 and 8, 4 and 9 and 5 and 10 show 6, 14, 21, and 28 days of incubation, respectively, (B) The LC was incubated with 1 mM peptide inhibitor containing 5 mM DTT (lanes 2-5) or without the peptide inhibitor (lanes 10-7) at 4° C. DTT, which does not have an effect on proteolysis, was added to maintain the peptide in monomer form. Lanes 2 and 10, 3 and 9, 4 and 8, and 5 and 7 show 6, 14, 21 and 28 days of incubation, respectively. In both panels A and B, lane 1 represents LC alone at day 0, and lane 6 has molecular weight markers (labels on left). The protein band IIIA (see FIG. 10) was faint in this experiment and was not captured in the photographic reproduction; therefore its location in the original gel is shown by arrows in the figure. Note that (a) presence (lanes 2-5, A) and absence (lanes 10-7, B) of EDTA had little effect on proteolysis of IA to IB and finally to IIIA, (b) TPEN (lanes 7-10, A) significantly reduced the rate of conversion of IA to IB and prevented formation of IIIA during the course of the experiment, and (c) the peptide inhibitor (lanes 2-5, B) drastically reduced the proteolysis of IA to IB and prevented the formation of IIIA.

FIG. 13. Scheme I. Steps in the self-proteolysis of BoNT/A LC in the absence of added zinc. Arrows show the sites of proteolysis. Full-length LC is denoted by IA. The fragments IB, IIIB, and IVC correspond to the fragment designations in FIG. 10. The primary event is the C-terminal truncation to form IB followed by cleavage between Y286 and G287 producing IIIA and IVC. The fragment IIIA in turn is further proteolyzed between Y251 and Y252 to generate IIIB. Lengths of the fragments (e.g., IV-K448) are based on mass determined by MALDI-MS and N-terminal amino acid sequence shown in Table 5. The C-terminal peptide E424-K448, although shown here as a single peptide for convenience, is in fact a mixture of several peptides (see Tables 4 and 5).

FIG. 14. Scheme II. Steps in the self-proteolysis of BoNT/A LC in the presence of added zinc. Arrows show the sites of proteolysis. The fragments IIIB, IVA, and IVB correspond to the fragment designations in FIG. 2. Unlike the steps shown in Scheme I, IA may bypass the C-terminal truncation and initial formation of IIIA but undergo proteolysis between Y251 and Y252 in directly forming IIIB. The fragment IVA is further cleaved into IVB. Although a C-terminal cleavage of IVB into IVC is possible, it was not observed here (see FIG. 11) this species in the presence of added zinc. See FIG. 11 and Scheme I for other explanations.

FIG. 15. SDS-PAGE of (A) LCA, (B) LCA+Belt, and (C) LCA+Xloc, expressed at 18° C., 30° C. and 37° C. Odd numbered lanes (1, 3, 5 and 7) are the soluble fractions and even number lanes (2, 4, 6 and 8) are the insoluble fractions. Lanes 7 and 8 are control cells with the plasmid lacking the insert. Arrows show the expressed product at 18° C. (soluble) and 37° C. (insoluble).

FIG. 16. HPLC elution profiles from HS column of LcA (A, B), LcA+Belt (C, D), LcA+Hn (E, F), and LcB (G,H) and from a Source S column of LcA (I, J).

FIG. 17. SDS-PAGE (A) and Western blots of purified LcA constructs using rabbit peptide sera against LcA (B), LcA+Belt (C) and LcA+Hn (D). Lanes from all figures are identical. Lane 1, Novex See Blue prestained molecular weight markers; Lane 2, purified BoNt-A; Lane 3, LcA-HIS; Lane 4, LcA-phosphate buffer; Lane 5, LcA-NaAcetate buffer; Lane 6, LcA+Belt; Lane 7, LcA+Hn, nicked; Lane 8, LcA+Hn, un-nicked; Lane 9, negative control pET24a construct, no insert; Lane 10, LcB.

FIG. 18. Purification of LcA, LcA+Belt, and LcA+Hn from E. coli cells.

### DETAILED DESCRIPTION OF THE INVENTION

In some embodiments the invention provides methods and nucleic acids for expressing Clostridium botulinum genes in other prokaryotes and eukaryotes. More specifically, the invention provides methods and nucleic acids for expressing botulinum neurotoxin (BoNT) light chains (LC) in Escherichia coli or Pichia pastoris. In order to be expressed in Escherichia coli or Pichia pastoris, the sequence of DNA encoding wild-type BoNT LC is engineered to replace some Clostridium codons that are rare or unrecognized in the host organism and to reduce the A+T content. The recombinant or synthetic DNA molecules of the invention are preferably designed with codon usage normally found in genes that are highly expressed in the host organism, e.g. Escherichia coli or Pichia pastoris. By selecting codons rich in G+C content, synthetic DNA molecules may also be designed to lower the A+T-rich base composition found in the Clostridial genes. According to the invention, a host cell is a cell of any organism other than Clostridium. Nonlimiting examples of host cells include gram negative bacteria, yeast, mammalian cells, and plant cells.

In some embodiments of the invention, upon expression of the DNA, a BoNT LC is produced in a heterologous host system by itself or as a fusion with another protein or a carrier. Proteins with which BoNT LCs may be fused include BoNT HCs, maltose-bonding proteins, other neurotoxins, neuropeptides, and autofluorescent proteins. A synthetic light chain gene may be genetically fused to a gene encoding a BoNT HC, producing recombinant botulinum toxin.

In some embodiments of the invention, BoNT LC is produced that is (i) substantially free of contaminating toxicity, (ii) moderately to highly soluble in aqueous media, (iii) stable for at least about six months at 4° C., (iv) catalytically active, (v) functionally active, or combinations thereof. In some embodiments of the invention, gram quantities of BoNT LC may be obtained per liter of culture medium. In some embodiments of the invention, a recombinant BoNT LC may reduce any immunological response that may result from the presence of binding proteins associated with the recombinant BoNT LC.

In some embodiments, the invention provides BoNT LC that substantially lacks catalytic activity as a zinc protease as measured by the SNAP-25 assay described in Examples 8, 17, and, 25 below. In some embodiments, the invention provides nucleic acids that encode recombinant BoNT LC substantially lacking catalytic activity as a zinc protease, wherein amino acids in or spatially near the active site are deleted, replaced or modified relative to wild-type native BoNT. Catalytically inactive BoNT LC may be fused with BoNT HC to form a mutant recombinant holotoxin. Such holotoxins may be used to carry molecules, e.g., drugs, into cholinergic nerve cells.

In some embodiments, this invention provides a nucleic acid comprising a nucleic acid sequence encoding the N-terminal portion of a full length botulinum neurotoxin (BoNT) selected from the group consisting of BoNT serotype A, BoNT serotype B, BoNT serotype C1, BoNT serotype D, BoNT serotype E, BoNT serotype F, and BoNT serotype G, wherein said nucleic acid is expressible in a recombinant organism selected from Escherichia coli and Pichia pastoris. In some preferred embodiments, the nucleic acid corresponds in length and encoded amino acid sequence to the BoNT light chain (LC). In some particularly preferred embodiments, the nucleic acid comprises a nucleic acid sequence selected from SEQ ID NO:4 (serotype A), SEQ ID NO:6 (serotype B), SEQ Id NO:8 (serotype C1), SEQ ID NO:10 (serotype D), SEQ ID NO:12 (serotype E), SEQ ID NO:14 (serotype F), SEQ ID NO:16 (serotype G), SEQ ID NO:22 (serotype B), SEQ Id NO:26 (serotype C1), SEQ ID NO:30 (serotype D), SEQ ID NO:34 (serotype E), SEQ ID NO:38 (serotype F), and SEQ ID NO:42 (serotype G).

In preferred embodiments, nucleic acids of the invention are synthetic nucleic acids. In some preferred embodiments, the sequence of the nucleic acid is designed by selecting at least a portion of the codons encoding BoNT LC from codons preferred for expression in a host organism, which may be selected from gram negative bacteria, yeast, and mammalian cell lines; preferably, the host organism is Escherichia coli or Pichia pastoris. The nucleic acid sequence encoding LC may be designed by replacing Clostridium codons with host organism codons that encode the same amino acid, but have a higher G+C content. Conservative amino acid substitutions are within the contemplation and scope of the invention. In preferred embodiments of the invention, a nucleic acid encoding a recombinant BoNT or fragment thereof is capable of being expressed in a recombinant host organism with higher yield than a second nucleic acid encoding substantially the same amino acid sequence, said second nucleic acid fragment having the wild-type Clostridium botulinum nucleic acid sequence.

Codon usage tables for microorganisms have been published. See e.g. Andersson SGE, Kurland CG, 1990, “Codon preferences in free-living microorganisms” Microbiol. Rev 54:198-210; Sreekrishna, 1993, “Optimizing protein expression and secretion in Pichia pastoris” in Industrial Microorganisms: Basic and Applied Molecular Genetics, Baltz, Hegeman, Skatrud, eds, Washington D.C., p. 123; Makofl A J, Oxer M D, Romanos M A, Fairweather N F, Ballantine S, 1989, “Expression of tetanus toxin fragment C in E. coli: high level expression by removing rare codons” Nuc. Acids Res. 17(24): 10191-10202. Table 3 of Skreekrishna is a chart depicting codon usage in Pichia pastoris. This table was generated by listing the codons found in a number of highly expressed genes in P. pastoris. The codon data was obtained by sequencing the genes and then listing which codons were found in the genes.

From such tables, it is clear that amino acid residues can be encoded by multiple codons. When constructing synthetic DNA molecules using P. pastoris codon usage, it is preferred to use only those codons that are found in naturally occurring genes of P. pastoris, and it should be attempted to keep them in the same ratio found in the genes of the natural organism. When the clostridial gene has an overall A+T richness of greater than 70% and A+T regions that have spikes of A+T of 95% or higher, they have to be lowered for expression in expression systems like yeast. Preferably, the overall A+T richness is lowered below 60% and the A+T content in spikes is also lowered to 60% or below. In preferred embodiments of the invention, maintaining the same codon ratio (e.g., for glycine GGG was not found, GGA was found 22% of the time, GGT was found 74% of the time, GGC was found 3% of the time) is balanced with reducing the high A+T content. In the construction of the DNA molecules of the invention, it is preferred to avoid spikes where the A+T content exceeds about 55%.

According to the invention, a spike may be a set of about 20 to about 100 consecutive nucleotides. A spike having a high A+T content greater than 80% or 90% may function as transcription termination sites in host systems, thereby interfering with expression. Preferred synthetic DNA molecules of the invention are substantially free of spikes of 50 consecutive nucleotides having an A+T content higher than about 75%. Alternatively, preferred synthetic DNA molecules of the invention are substantially free of spikes of 75 consecutive nucleotides having an A+T content higher than about 70%. Alternatively, preferred synthetic DNA molecules of the invention are substantially free of spikes of 100 consecutive nucleotides having an A+T content higher than about 60%.

A synthetic DNA molecule of the invention designed by using E. coli codons is expressed fairly well in P. pastoris. Similarly, a synthetic gene using P. pastoris codons also appears to be expressed well in E. coli.

In some embodiments, this invention provides an expression vector comprising a nucleic acid of this invention, whereby LC is produced upon transfection of a host organism with the expression vector. Another embodiment of this invention provides a method of preparing a polypeptide comprising the BoNT LC selected from the group consisting of BoNT serotype A, BoNT serotype B, BoNT serotype C, BoNT serotype D, BoNT serotype E, BoNT serotype F, and BoNT serotype G, said method comprising culturing a recombinant host organism transfected with an expression vector of this invention under conditions wherein BoNT LC is expressed. Preferably, the recombinant host organism is a eukaryote. In another preferred embodiment, the method of this invention further comprises recovering insoluble protein from the host organism, whereby a fraction enriched in BoNT LC is obtained. E. coli is a preferred host for expressing catalytically active (i.e., proteolytically active) LC. Pichia pastoris is a preferred host organism for expressing inactive or mutated LC. Pichia pastoris has SNARE proteins which probably get inactivated by catalytically-active LC.

In some embodiments, the invention provides an immunogenic composition comprising a suitable carrier and a BoNT LC selected from the group consisting of BoNT serotype A, BoNT serotype B, BoNT serotype C, BoNT serotype D, BoNT serotype E, BoNT serotype F, and BoNT serotype G. Preferably, the immunogenic composition is prepared by culturing a recombinant organism transfected with an expression vector encoding BoNT LC. More preferably, the immunogenic composition is prepared by a method wherein an insoluble protein fraction enriched in BoNT LC is recovered from said recombinant organism. More preferably, the immunogenic composition is prepared by the method of Example 30.

According to some non-limiting embodiments, the invention provides reagents and compositions that are useful for developing therapeutic interventions against BoNT. For example, the recombinant BoNT nucleic acids and polypeptides of the invention may be used to screen for botulinum neurotoxin inhibitors.

In some embodiments, the invention provides therapeutic agents for clinical disorders such as dystonias, spasticity, and pain. According to these embodiments, the agents may be prepared by first expressing and purifying BoNT LC independently of any portion of the heavy chain. The BoNT LC so produced is then fused to the heavy chain or fragments thereof, e.g., HN and HC. Alternatively, BoNT LC may be coexpressed and/or copurified with BoNT HC or fragments thereof and then fused to BoNT HC or fragments thereof. These agents may be used in clinical (human) or veterinary (non-human animal) applications.

In some embodiments, the invention provides agents that may be useful for treating disorders associated with cholinergic nerve function, SNAP-25, VAMP, syntaxin or combinations thereof. In some embodiments, the invention provides agents that may be useful for reducing any immunological response that may result from the presence of binding proteins associated with the agents. For example, the native BoNT holotoxin is highly immunogenic and some patients become refractory to continued treatment with it over time as their protective antitoxin titer rises. The efficacy of holotoxin-based drugs (e.g., BOTOX, Myobloc/Neurobloc, Dysport) may be improved by pretreating patients having a high titer of anti-holotoxin antibodies with a holotoxin fragment such as Lc, Hn, or Hc. These fragments may bind the anti-holotoxin antibodies making them unavailable for binding the subsequently administered holotoxin. This may work for a short time (months to a few years) realizing eventually that the antibody level may be built up so much that the drug can no longer be effective even with the addition of fragments. At this point in time, the patients will have to use a different serotype toxin drug or a chimera of the toxin (i.e., mixing toxin domains).

In further embodiments, the invention provides an immunogenic composition comprising a suitable carrier and a BoNT LC selected from the group consisting of BoNT serotype A, BoNT serotype B, BoNT serotype C, BoNT serotype D, BoNT serotype E, BoNT serotype F, and BoNT serotype G. Preferably, the immunogenic composition is prepared by culturing a recombinant organism transfected with an expression vector encoding BoNT LC. More preferably, the immunogenic composition is prepared by a method wherein an insoluble protein fraction enriched in BoNT LC is recovered from said recombinant organism.

The LC is present in immunogenic compositions of the invention in an amount sufficient to induce an immunogenic response thereto.

Two of the major advantages of the recombinant botulinum neurotoxins and fragments of the invention are the safety and high yields possible. First, the recombinantly produced botulinum neurotoxin (rBoNT) protein fragments are completely nontoxic and are, thus, very safe. The fermentation of the host cell harboring the rBoNT gene (e.g., Escherichia coli or Pichia pastoris) does not require the high biological containment facilities presently needed to ferment the spore-forming Clostridium botulinum required for the production of the neurotoxin light chains. Second, synthetic DNA molecules of the invention can be placed in high expression systems and used to make much larger quantities of the BoNT fragments than toxin produced by the parent organism, Clostridium botulinum. Thus, there may be immense cost savings because it will be easier and safer to produce much larger quantities of the proteins for various uses including vaccination.

Synthetic DNA molecules as described herein may be transfected into suitable host organisms to create recombinant production organisms. Cultures of these recombinant organisms can then be used to produce recombinant BoNT fragments or holotoxins. Exemplary techniques for transfection and production of BoNT fragments are shown in the Examples. Alternative techniques are well documented in the literature See, e.g., Maniatis, Fritsch & Sambrook, “Molecular Cloning: A Laboratory Manual” (1982); Ausubel, “Current Protocols in Molecular Biology” (1991); “DNA Cloning: A Practical Approach,” Volumes I and II (D. N. Glover, ed., 1985); “Oligonucleotide Synthesis” (M. J. Gait, ed., 1984); “Nucleic Acid Hybridization” (B. D. Hames & S. J. Higgins, eds., 1985); “Transcription and Translation” (B. D. Hames & S. J. Higgins, eds., 1984); “Animal Cell Culture” (R. I. Freshney, ed., 1986); “Immobilized Cells and Enzymes” (IRL Press, 1986); B. Perbal, “A Practical Guide to Molecular Cloning” (1984), and Sambrook, et al., “Molecular Cloning: a Laboratory Manual” (1989). Such techniques are explained fully in the literature. Modification of these techniques within the scope of this invention is within the skill in the art.

Recombinant forms of botulinum neurotoxin light chain may be useful in one or more of the following applications: strabismus and other disorders of ocular motility, dystonia, blepharospasm, cervical dystonia, oromandibular dystonia, laryngeal dystonia (spasmodic dysphonia), limb dystonia, hemifacial spasm and other facial dyskinesias, tremors of the head and hands, eyelid, cervical, and other tics, spasticity (e.g. anal), Stiff-Person syndrome, bladder dysfunction (e.g. in patients with spinal-cord injury), segmental myoclonus and other hyperkinetic disorders, cosmetic treatment of glabelar frown lines and other facial wrinkles, and all conditions characterized by hyperactivity of the lower motor neuron. See Cardoso and Jankovic, 1995, “Clinical use of botulinum neurotoxins” Curr Top Microbiol Immunol. 195:123-41 and references cited therein. The light chain may further be used to control autonomic nerve function (U.S. Pat. No. 5,766,605) or tiptoe-walking due to stiff muscles common in children with cerebral palsy, according to findings published in the November 2001 issue of Pediatrics.

Absolute contraindications to the use of BONT are allergy to the drug and infection or inflammation at the proposed injection site whereas myasthenia gravis, Eaton-Lambert syndrome, motor neuron disease, and coagulopathy are relative contraindications (National Institutes Of Health Consensus Development Conference Statement On Clinical Use Of Botulinum Toxin 1991; Report Of The Therapeutics And Technology Assessment Subcommittee Of The American Academy Of Neurology 1990). Safety for use during pregnancy and lactation has not been firmly established (National Institutes Of Health Consensus Development Conference Statement On Clinical Use Of Botulinum Toxin 1991).

The invention contemplates isoforms of the light chain as well as chimeras with other domains of the toxin or other proteins. In other words, gene fragments with DNA sequences and amino acid sequences not identical to those disclosed herein may be discovered in nature or created in a laboratory. The invention contemplates the production of any protein or polypeptide that has biological activity/functionality similar to the wild-type botulinum neurotoxin light chain, e.g. cell binding, translocation across membrane, catalytic activity sufficient to inactivate critical proteins in a cell involved with protein trafficking, release of various chemical transmitters (i.e., acetylcholine, glutamate, etc), hormones, etc.

For example, the light chain and translocation domain may be combined with a protein or peptide that targets a different receptor and/or cell-type. In addition, the invention contemplates therapeutic delivery of synthetic DNA molecules of the invention to cells via viral vectors such as adenovirus or other gene therapy techniques.

### EXAMPLES

In order to facilitate a more complete understanding of the invention, a number of nonlimiting Examples are provided below for illustration purposes only. To advance these purposes, the Examples are arranged in four sets: Examples 1-13, Examples 14-20, Examples 21-29, and Example 30.

### Example 1

Chemicals, Buffers, and Reagents

Buffer T (20 mM Tris-HCl, pH 9.2) and buffer G (50 mM sodium glycine, pH 9.0) were used as indicated. SKL (sodium N-lauryl sarcosine or sarkosyl) was from Sigma. Highly purified (>95%) full-length BoNT/A was purchased from List Biologicals (Campbell, Calif.). Rabbit polyclonal antibodies against a 16-residue N-terminal sequence (PFVNKQFNYKDPVNGV; SEQ ID NO:1) of the BONT/A LC were produced and affinity purified by Research Genetics (Huntsville, Ala.). Peroxidase-coupled goat anti-rabbit and anti-mouse IgG (H+ L) and ABTS substrate were from Kirkegaard Perry Laboratories (Gaithersburg, Md.). Oligonucleotides, designed for E. coli codon usage (Anderson and Kurland, 1990) and ranging in size from 70 to 100 nucleotides, were synthesized by Macromolecular Resources (Fort Collins, Colo.).

### Example 2

Construction and Expression of a Synthetic DNA Encoding rBoNT/A LC

The DNA encoding the enzymatic LC domain of BoNT/A was assembled from three segments, a 335-base pair (bp) Sal I-Sph I fragment, a 600-bp Sph I-Kpn I fragment, and a 460-bp Kpn I EcoR I fragment. To construct the first segment, six oligonucleotide pairs were annealed, ligated, and, after PCR amplification, inserted into pGEM3Zf at Sal I-Sph I restriction enzyme sites. The second segment was built by annealing and ligating eight oligonucleotide pairs, followed by its amplification and insertion into the Sph I and Kpn I sites of pGEM3Zf. The final segment was constructed by annealing and ligating six oligonucleotide pairs, followed by its amplification and insertion into the Kpn I-EcoR I sites of pGEM3Zf. Nucleotide sequencing of gene fragments in pGEM3Zf was performed to identify clones in each group with minimal misincorporations. In vitro mutagenesis was performed to correct the misincorporations in the BoNT/A LC minigene fragments. Directional gene assembly via 600-bp and 460-bp fragments in pGEM3Zf was followed by the insertion of the 335-bp fragment.

In the design of the synthetic DNA, the 5′ oligonucleotide for amplifying the gene's 5′ terminus consisted of an anchored Sal I site followed by an EcoR I site and an Nco I site to facilitate directional subcloning into the E. coli expression vector, pET24d. The 3′ oligonucleotide contained a hexahistidine tag with a thrombin protease cleavage site for creating a carboxyl-terminal removable histidine tag. The 3′ end also included the restriction enzyme sites for BamH I and EcoR I.

The full-length gene was excised from pGEM3Zf 5 with an Nco I-EcoR I and subcloned into a similarly digested pET24d vector. The resulting ligated construct was used to transform E. coli BL21(DE3) cells. Two clones were assayed for their ability to express rBoNTA LC. Single colonies were inoculated into 5 ml of Luria broth (LB) containing 50 μg/ml of kanamycin and grown overnight at 37° C. The overnight cultures (500 μL) were used to inoculate 50 ml of LB containing 50 μg/ml of kanamycin. When the cultures reached OD₆₀₀ of 0.8, induction was initiated by addition of isopropyl-β-D-thiogalactoside (IPTG) (final concentration, 1.0 mM). The cultures were induced for 2 hr at 37° C., harvested, and analyzed for expressed products on SDS-PAGE.

Results

A synthetic DNA encoding rBoNTA LC was designed with E. coli codon usage, constructed, and expressed in E. coli. The native nucleic acid sequence from C. botulinum type A NTCC 2916 (Thompson et al., 1990) was used as the template for preparing synthetic LC sequences of the invention.

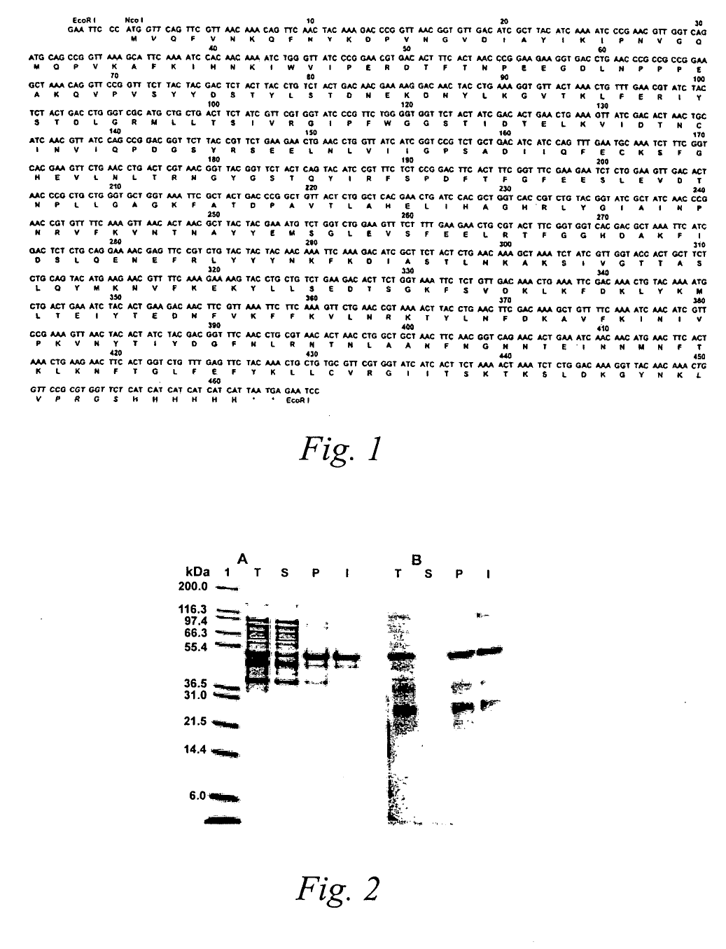

At the 5′ end of the DNA, an Nco I restriction enzyme site was employed as a cloning site and palindrome to provide an initiation codon. The use of this Nco I site necessitated the use of a filler codon (GTT) between the Met initiation codon (ATG) and the codon (CAG) specifying the first amino acid residue in the LC (i.e., Q). This resulted in the introduction of one extra amino acid, Val, as the N-terminal residue (after the initiating Met). This extra and new amino acid, however, did not interfere with expression or activity. The length of the LC (448 residues) to be expressed was chosen from the sequence of amino acids around the nicking site (DasGupta and Dekleva, 1990) (FIG. 1). At the C-terminal end (i.e., DKGYNK; residues 444-449 of SEQ ID NO:5), a hexa-His tag was incorporated for affinity purification and a thrombin cleavage site (LVPRGS; residues 450-455 of SEQ ID NO:5) was incorporated for removing the hexa-His tag. The expressed protein therefore contained a total of 461 (1+448+6+6) residues (FIG. 1 and SEQ ID NO:5). The synthetic gene thus constructed in pET24d vector was highly and efficiently expressed in E. coli, accounting for about 25% of the total protein (FIG. 2).

### Example 3

Fermentation

A frozen stock seed culture of recombinant E. coli harboring the synthetic DNA encoding the LC of BoNT/A was grown at 37° C. to an OD₆₀₀ of 2.682 in a shake flask containing 100 ml of the following defined medium: casamino acids (1.4 g/L); yeast extract (2 g/L); (NH₄)₂SO₄ (1.85 g/L); K₂HPO₄ (30 g/L); MgSO₄.7H₂O (2 g/L); thiamine.HCl (0.015 g/L); glucose (18.1 g/L); trace elements solution (3 ml/L) consisting of FeCl₃.6H₂O, 27 g; ZnCl₂.4H₂O, 1.3 g, CoCl₂.H₂O, 2 g; Na₂Mo₄.2H₂O, 2 g; CaCl₂.2H₂O, 1 g; CuCl₂.2H₂O, 1 g; H₃BO₃, 0.5 g; distilled H₂O, 1000 ml; and HCl, 100 ml. In addition, 0.0156 g/L of ZnCl was added to trace minerals to make the concentration of Zn five times greater in the shake flask and fermentor. Kanamycin (50 μg/L) was added as an antibiotic. The shake flask culture was used to inoculate a 5-L BioFlo III fermentor (New Brunswick Scientific, Edison, N.J.) containing 4.3 L of the medium described above. Later in the growth (5.5 hr), 14.1 g/L of casamino acids was added and a glucose feed was initiated to maintain a glucose concentration of 1 g/L. Growth continued for 8 hr until an OD₆₀₀ of 49.9 was reached. Cell induction was then initiated at this time by adding IPTG (final concentration, 1.5 mM). Induction continued for 4 hr after adding IPTG, and cells (OD₆₀₀ of 112.62) were harvested by centrifugation (Beckman, Palo Alto, Calif.) at 7000 rpm for 15 min at 4° C. Cells were washed with cold 0.9% saline and centrifuged at 7000 rpm for min and frozen at −70° C. Wet cell yield was 58 g/L.

### Example 4

Extraction and Purification of Light Chain as Inclusion Bodies

In a typical preparation, 12 g of E. coli cells was suspended in a total volume of 30 ml of buffer T containing 5 mM MgCl₂, 1.5 mM PMSF, 10 mM β-mercaptoethanol, and 2 mg of DNase. The cell suspension was subjected to 10 cycles of 2-min sonication (at 60% power in a Fisher Model 300 Sonic Dismembrator) and 2-min cooling on ice. After centrifugation for 15 min at 10,000×g, the supernatant was discarded. The pellet was suspended in 30 ml the above buffer. The cycle of sonication and centrifugation was repeated five more times; MgCl₂ and DNase were omitted from the buffer during the last two cycles. The resulting pellet contained the rBoNT/A LC, that appeared ˜70% pure by SDS PAGE (FIG. 2). The pellet was stored at 4° C. as a white suspension in 15 ml of buffer T containing 1.5 mM PMSF and 10 mM β-mercaptoethanol.

Results

The expressed LC appeared exclusively in the insoluble pellet fraction (FIG. 2). Including MgCl₂ and DNase in the cell suspension ensured a clean separation of the pellet from the supernatant after sonication and centrifugation. The white suspension of the purified BoNT/A LC migrated as a 52-kDa band and appeared to be ˜70% pure on SDS-PAGE (FIG. 2A), as determined by densitometric analysis. Minor contaminant bands with ˜100-kDa, 37-40 kDa, and ˜25 kDa also reacted with the antibody in the Western blot (FIG. 2B). While fragments smaller than 50 kDa may have arisen from proteolysis of the LC (DasGupta and Foley, 1989), the origin of the 100-kDa species in the reducing SDS-PAGE gels is not clear since the species also reacts with the affinity-purified antibodies against a small sequence of the LC. Molecular mass determination by MALDI-MS gave 52.774 (±50) kDa as the predominant species along with minor species of 106.028 (±100) kDa and 25.00 (±25) kDa. Amino acid sequence determination of the LC identified V-Q-F-V-N-K-Q (residues 2 to 8 of SEQ ID NO:5) as the amino-terminal sequence, as expected for the constructed gene (FIG. 1) and identical (with the exception of the penultimate valine) to that of the published sequence of BoNT/A (Thompson et al., 1990).

### Example 5

Solubilization of the Inclusion Bodies to Obtain Active rBoNT/A LC

In a typical experiment, 0.75 ml of the white rBoNT/A LC suspension (from an equivalent of 600 mg of wet cells) was centrifuged in a 2-ml Eppendorf tube and the supernatant was discarded. The pellet was suspended by mild sonication in 0.9 ml of 50 mM Tris-HCl, pH 9. A 20% solution (0.9 ml) of SKL in water was added to the suspension at room temperature and was mixed by inversion several times. Within 2 min, the pellet became completely soluble. Any remaining turbidity was cleared by further diluting with 50 mM Tris-HCl, pH 9.0, or was removed by centrifugation. The SKL-solubilized LC was dialyzed against 200 volumes of buffer G containing 1 mM DTT with one to two daily changes at 4° C. for 1 week. The yield of the soluble rBoNT/A LC was 12 mg (3.9 mg/ml), which was stored in a glass tube at 4° C.

Results

The purified inclusion bodies were solubilized in 10% SKL and the SKL was removed by dialysis against buffer G containing 1 mM DTT (see Section 2). The use of a 10% SKL solution ensured solubilization within 2 min of incubation, and the LC solution was immediately subjected to extensive dialysis to remove the detergent. Starting with an equivalent of 600 mg of the wet E. coli cells, 12 mg of the soluble LC was obtained, corresponding to 20 mg LC per gram of wet cells. This corresponds to a yield of 1.16 g of the pure protein per liter of cell culture.

### Example 6

Properties of the Purified BoNT/A LC

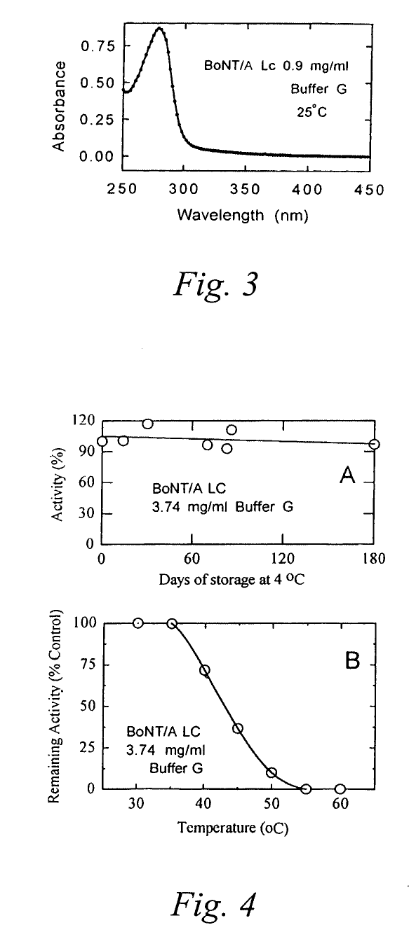

The UV-visible absorption spectrum (FIG. 3) shows the rBoNT/A LC with a single maximum at 278 nm as a simple protein. Although a number of minor band were observed in the SDS-PAGE gel (FIG. 2), absence of any other absorbance bands in the UV-visible range suggests the absence of any nonmetal cofactor in the preparation. The LC was expressed as a C-terminally His-tagged protein. In the presence of 6 M GuHCl, the rBoNT/A LC was bound to Ni-resin and was eluted with immiadzole-containing buffers as a more purified form. Without GuHCl, the rBoNT/A LC did not bind to Ni-resin. This result suggests that the LC retained the His-tag after expression and purification, but in the absence of GuHCl, the His-tag was not exposed to solvent to chelate with the Ni-resin. Because the rBoNT/A LC had catalytic properties comparable to those of the dicchain (see below), removal of the His-tag from the purified protein was not attempted.

The purified LC was stable for at least 6 months when stored at 4° C. in buffer G containing 1 mM DTT (FIG. 4A). During this period, the protein remained fully soluble, did not show any degradation as analyzed SDS-PAGE, and retained its initial catalytic activity. An LC preparation obtained by prolonged solubilization in 0.5% SKL at room temperature, however, precipitated after 3 months of storage at 4° C. and lost most of its initial catalytic activity. The LC (1 mg/ml of 50 mM Na-phosphate) precipitated from solution below pH 8 either at 4° C. or at 25° C. Thermal stability of the LC (3.74 mg/ml of buffer G containing 1 mM DTT and 50 μM ZnCl₂) was investigated by incubating aliquots for 45 min at various temperatures. After cooling on ice for 45 min, the catalytic activities in the supernatants were measured. The midpoint of thermal unfolding Tₘ as measured by activity was 43° C. (FIG. 4B). At room temperature, increasing concentration of MgCl₂ also precipitated the LC from solution: at 6 mM MgCl₂, >80% of the LC precipitated.

### Example 7

Preparation of Apo-rBoNT/A LC

One milliliter of rBoNT/A LC (2.73 mg) was dialyzed overnight against 250 ml of buffer G containing 5 mM EDTA and 1 mM DTT. EDTA was removed by further dialysis for 60 hr against three changes of 250 ml of buffer G containing 1 mM DTT.

### Example 8

Assay of Proteolytic Activity of BoNT/A LC

BoNT/A cleaves the glutamyl-arginine bond between residues 197 and 198 of the 206-residue SNAP-25. Schmidt and Bostian (1995) showed that a synthetic 17-residue peptide representing residues 187-203 of SNAP-25 was sufficient for detecting endopeptidase activity of BONT/A and allowing routine assay for the neurotoxin activity. The peptide thus probably mimics the structure of SNAP-25 in vivo (Bi et al., 1995). The same peptide was used in an identical method to assay the proteolytic activity of the BONT/A LC.

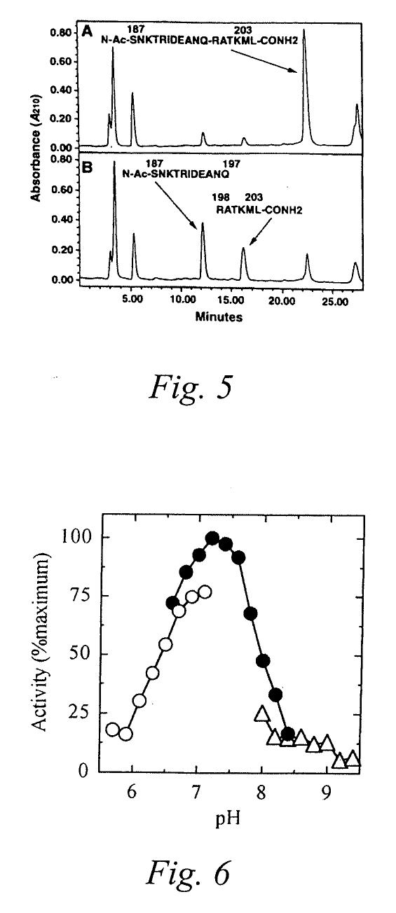

The assay is based on HPLC separation and measurement of the nicked products from a 17-residue C-terminal peptide of SNAP-25 (FIG. 5), corresponding to residues 187-203, which is the minimum length required for BoNT/A proteolytic activity (Schmidt and Bostian, 1995, 1997). Unless otherwise noted, a 0.03-ml assay mixture containing 0.8-1.0 mM substrate, 0.25 mM ZnCl₂, 5.0 mM DTT, 50 mM Na-HEPES buffer (pH 7.4), and BONT/A LC was incubated at 37° C. for 15-80 min. The amounts of uncleaved substrate and the products were measured after separation by reverse-phase HPLC (Waters) on a Hi-Pore C 18 column, 0.45×25 cm (Bio-Rad Laboratories, Hercules, Calif.) with the Millennium software (Waters) package. Solvent A was 0.1% TFA and solvent B was 70% acetonitrile/0.1% TFA. The flow rate was 1.0 ml/min at 25° C. After the column was equilibrated with 10% B, the sample was injected, and the column was held at 10% B for 2.5 min. A linear gradient to 36% B over 21 min was followed by 100% B for 6 min. Kinetic parameters for the synthetic substrate were calculated from Lineweaver-Burk plots of activity with peptide concentrations from 0.26 to 1.7 mM.

Catalytic Activity of the LC

The BoNT/A LC is zinc-endopeptidase specific for the cleaving the peptide bond between residues 197 (Glu) to and 198 (Arg) of SNAP-25. Incubating the 17-mer synthetic peptide representing residues 187-203 of SNAP-25 with the LC at 37° C. for 5-200 min generated only two peptides (FIG. 5). That no other peptide fragments were generated by this prolonged incubation proves that the contaminants present in the LC preparation were devoid of any proteolytic activity. Incubating the LC with BSA also failed to produce any proteolytic fragment. In contrast to the BoNT/A dichain, whose activity ruin is greatly enhanced by BSA (Schmidt and Bostian, 1997), the rate of cleavage of the synthetic peptide substrate was unaffected by the presence of BSA.

Proteolytic activity of the purified rBoNT/A LC linearly increased with the increasing amount of the LC in the reaction mixture. The time course of activity (at 0.8-1.0 mM substrate concentration), however, was not linear, but progressively declined, possibly due to a high Kₘ for the substrate peptide (see below). Therefore, routine assays depended on initial activities representing <30% substrate conversion.

Substrate Kₘ for the LC was fourfold lower than that reported for the dichain (Schmidt and Bostian, 1995). This may be due to shielding of the active site by a ‘belt’ from the translocation domain (Hₙ) in the dichain neurotoxin (Lacy et al., 1998; Lacy and Stevens, 1999). Thus, the ‘belt’ may pose a steric hindrance for substrate binding by the dichain (high Kₘ). Nonetheless, the catalytic efficiency k_{cat}/Kₘ of the free rBoNT/A LC was somewhat higher than that of the dichain.

Optimum pH, Salts, and Buffers

An optimum pH of 7.2 for the proteolysis of the synthetic substrate by the rBoNT/A LC was determined by assaying in three different buffer systems (0.1M) ranging in pH from 5.0 to 9.0 (FIG. 6). For comparison, the optimum pH values of BoNT/B and tetanus neurotoxin, two members of the clostridial neurotoxin family, are 6.5-7.0, and 6.5-7.5, respectively (Foran et al., 1994). Tris-HCl appeared to have an inhibitory effect on proteolysis, presumably due to chelation with the zinc at the active site. The activity at pH 7.4 was 25% higher in a 50 mM HEPES buffer than in 100 mM HEPES. Adding 50 mM NaCl, KCl, or NaPO₄ (pH 7.4) to the standard reaction mixture reduced activity 40-50%. Thus, high salt concentrations inhibited the proteolytic reaction.

Effect of Metals and Thiol Reagents on Activity

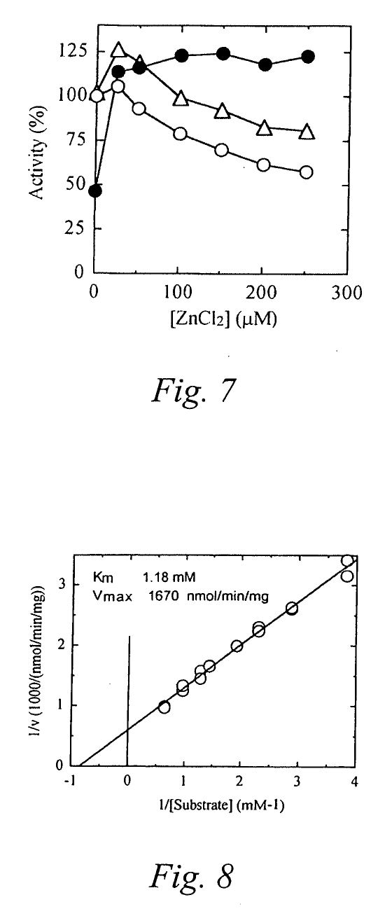

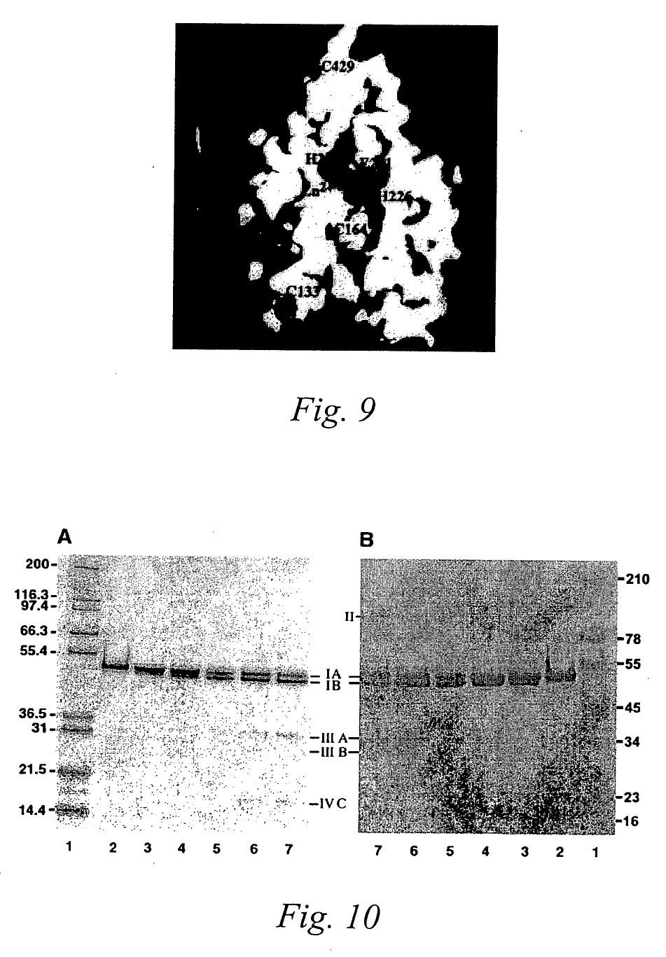

BoNT/A LC is a zinc-endopeptidase. Activity of the rBoNT/A LC was completely inhibited by including the metal chelator EDTA (1 mM) in the reaction mixture (Table 1). Adding low concentrations of ZnCl₂ (1-50 μM) in the assay mixture slightly stimulated the activity (5%-10%) and higher concentrations of ZnCl₂ inhibited the activity (FIG. 7). The results suggest that the active site should be almost saturated with Zn²⁺ for optimum activity. The metal was tightly bound to the active site of the LC, as the extraction, purification, or dialysis buffers were devoid of Zn²⁺. Like Zn²⁺, other divalent metal ions, notably, MnCl₂ and NiSO₄, also inhibited the LC reaction to various extents in the absence of added thiol (Table 1). Adding 5 mM DTT to the reaction mixture neutralized the inhibitory effect of Zn²⁺ (FIG. 7).

Neurotoxic or proteolytic activity of the dichain BONT/A probably requires an initial reduction of the disulfide bond between the LC and the HC (de Paiva et al., 1993). Therefore, the proteolytic assay mixture of BONT/A with the synthetic or natural substrates were supplemented with 5-10 mM DTT (Washbourne et al., 1997; Schmidt and Bostian, 1995, 1997). In the absence of Zn²⁺, 5 mM DTT in the reaction mixture significantly inhibited the activity of the LC (Table 1 and FIG. 7). Similarly, L-cys, dithioerythreitol, and glutathione inhibited the activity to various extents, while β-mercaptoethanol stimulated the activity in the absence of added Zn²⁺. These results were unexpected as the LC does not possess any disulfide bonds and the invariant Cys responsible for the interchain disulfide is far from the active site. One explanation for these effects is the formation of a mixed disulfide between a protein thiol and the exogenous thiol. To investigate the importance of a protein Cys residue on activity, several sulfhydryl reagents were incubated in the proteolytic assay mixture (Table 1). Both HgCl₂ and p-Cl-mercuric benzoate completely abolished the activity of LC. Preincubating the LC with these two reagents, then diluting with the proteolytic reaction mixture, also gave the same results. These results suggest the presence of a protein thiol in the vicinity of the active site of the LC.

TABLE 1
Effect of Metal Ions and Thiols and Thiol
Reagents on the Activity of the rBoNT/A LC

| | Concentration | % | Metal | Concentration | % |
|---|---|---|---|---|---|
| Thiol reagent | (mM) | Activity | reagent | (mM) | Activity |
| None^{a} | | 100 | EDTA | 1 | 00 |
| Dithiothreitol | 5 | 45 | ZnCl₂ | 0.25 | 60 |
| Dithioerythreitol | 5 | 60 | — | 1 | 10 |
| β-Mercaptoethanol | 5 | 120 | — | 0.25 | |
| Glutathione, reduced | 5 | 75 | +Dithiothreitol | 5 | 125 |
| Glutathione, oxidixed | 5 | 75 | MnCl₂ | 1 | 40 |
| S-Nitrosoglutathione | 5 | 55 | MgCl₂ | 1 | 90 |
| L-Cysteine | 5 | 20 | CaCl₂ | 1 | 75 |
| p-Cl-Mercuribenzoate | 0.050 | 00 | FeCl₃ | 1 | 35 |
| Mercuric chloride | 0.013 | 00 | CoCl₂ | 1 | 90 |
| Iodoacetamide | 10 | 80 | CuSO₄ | 1 | 95 |
| | | | NiSO₄ | 1 | 55 |

^{a}The reaction mixture contained only the substrate and the rBoNT/A Lc. Other conditions are as described in Examples 8-20.

Steady-State Kinetic Parameters

The dependence of reaction rates on the substrate concentration was determined at 0.26-1.7 mM substrate at pH 7.4. A double reciprocal plot of the reaction rates versus substrate concentrations (FIG. 8) yielded a Kₘ of 1.18 mM and a Vₘₐₓ of 1670 (equivalent to 2390 considering a 70% pure LC) nmol/min/mg LC (k_{cat}=1.39/sec or 1.99 if 70% pure). For comparison, the maximum rate of cleavage of the peptide substrate by the native, dichain toxin is reported to be 1900 nmol/min/mg (k_{cat}=4.7/sec), while the Kₘ is 5 mM (Schmidt and Bostian, 1997). The lower Kₘ for the LC may be due to a more exposed active site in the free LC than in the LC of the dichain, where the active site is shielded from the solvent by elements of the membrane-spanning domain H_{N} (28-29). The catalytic efficiency k_{cat}/Kₘ of the rBoNT/A LC, 1.18 (1.69 if 70% pure), is thus higher than that of the dichain, 0.94 (Schmidt and Bostian, 1995, 1997).

Apo-BoNT/A LC

The rBoNT/A LC was incubated with the metal chelator EDTA and after extensive dialysis, the activity of the apo-BoNT/A LC was measured in the standard reaction mixture. In the absence of any exogenous Zn²⁺ or thiol, the preparation had 17% activity of the holo-BoNT/A LC from which the apoprotein was made (Table 2). This result suggests that the bound Zn²⁺ was not completely removed by the EDTA treatment and dialysis. Nonetheless, adding 5 mM DTT and 250 μM ZnCl₂ to the assay mixture restored 70% of the activity of the holo-LC. Moreover, in the presence of 5 mM DTT and 250 μM MnCl₂, MgCl₂, or CaCl₂, 20-30% of the original activity was restored.

TABLE 2
Activities of the Apo-BoNT/A LC With and Without Addition
of Divalent Metal Ions to the Reaction Mixtures

| | Divalent | % | % Activity |
|---|---|---|---|
| LC form | metal | Activity | recovered^{a} |
| Holo-LC | +Zn²⁺ | 100 | — |
| Apo-LC | +None | 15 | — |
| | +Zn²⁺ | 70 | 65 |
| | +Mn²⁺ | 20 | 10 |
| | +Mg²⁺ | 20 | 10 |
| | +Ca²⁺ | 30 | 20 |
| | +Fe²⁺ | 0 | — |

^{a}Represents percentage of the lost activity of Zn-free apo-rBoNT/A LC that was recovered by adding the indicated metal ions.

### Example 9

Vaccination of Animals

Purified rBoNTA LC was tested for its ability to elicit protective immunity in Cr1:CD-1 (ICR) male mice (Charles River) weighing 16-22 g. Two concentrations of recombinant LC (5 and 15 micrograms) with and without adsorption to a 0.2% Alhydrogel (Superfos Biosector, Kvisgaard, Denmark) were administered in 0.9% saline in a total volume of 100 μl. Groups of 10 mice including a naive control (saline alone) received three doses of LC at 0, 2, and 4 weeks. Mice were bled from the retroorbital sinus 12 days postvaccination and their antibodies assayed for titers to toxin. Animals were challenged with native BoNT/A dichain toxin 15 days postvaccination.

The animal room was maintained at 21±2° C. with a relative humidity 30-70%, a 12/12-hr light/dark cycle with no twilight, and 10-15 air changes/hour. Mice were housed in solid-bottom, polycarbonate Micro-Isolator™ cages (Lab Products, Inc., Seaford, Del.) with paper chip bedding (Alpha-Dri™, Shepherd Specialty Papers, Inc., Kalamazoo, Mich.) and provided food (Harlan Teklad diet No. 7022, NIH-07) and water ad libitum. All procedures were reviewed and approved by the Institutional Animal Care and Use Committee and performed in an AAALAC International-accredited facility in accordance with recommendations in the Guide for the Care and Use of Laboratory Animals, 1996 (National Academy Press, National Academy of Sciences, Washington, D.C.).

### Example 10

ELISA

Highly purified (>95%) BoNT/A toxin was diluted to 2 μg/ml in phosphate-buffered saline (PBS), pH 7.4 (Sigma Chemical Co., St Louis, Mo.) and was dispensed (100 μl/well) into microtiter plates (Immulon 2, Dynatech Laboratories, Chantilly, Va.). The plates were incubated overnight in a humidity box at 40° C. Five percent skim milk (Difco, Detroit, Mich.) in PBS with 0.01% Thimerosal® was used to block nonspecific binding and as an antibody diluent. The plates were washed with PBS plus 0.1% Tween 20 between each step. Mouse sera were initially diluted 1:100 and then diluted fourfold for a total of eight dilutions (1:100 to 1:1,600,000). Diluted sera were added in duplicate to toxin-coated wells (100 μl/well). The secondary antibody was horseradish peroxidase-conjugated, goat anti-mouse IgG diluted 1:1000. The primary and secondary antibodies were incubated 90 and 60 min, respectively at 37° C. ABTS substrate (100 μl/well) was added as the color developer. The plates were incubated at room temperature for 30 min. The absorbance was measured with a microplate reader at 405 nm. A mouse monoclonal antibody, 5BA2.3, was used as the positive control in each assay; naive mouse serum was added as a negative control in each assay. The titer was defined as the geometric mean of the ELISA titer to BoNT/A toxin.

### Example 11

Biological Effects of the rBoNT/A LC

LC prepared from dichain BoNTs always had residual toxicity due to some contaminating dichain forms (Maisey et al., 1988). To demonstrate and confirm that the rBoNT/A LC was nontoxic, 5-15 μg of the LC was injected per mouse, a dose that was 15,000-45,000 times higher than an equivalent lethal dose of the BoNT/A dichain. Table 3 shows that all the mice survived three successive injections. All of their antisera had high titers against BoNT/A, but these antibodies failed to protect the animals upon subsequent challenge with relatively low doses (10²LD₅₀) of the toxic BoNT/A dichain. Even when the ELISA titers were boosted 20-fold by using the aluminum hydroxide adjuvant, the animals were not immune to modest levels of BoNT/A challenge (Table 3). Comparable vaccination with BoNT/A Hc protected animals from challenge with as high as 10⁶ LD₅₀ (Smith, 1998). These results clearly demonstrate that the rBoNT/A LC was nontoxic to the animals and confirms earlier observations that LC does not possess any neutralizing epitope(s) (Chen et al., 1997; Dertzbaugh and West, 1996).

TABLE 3
Survival of Mice After Vaccination with the rBoNT/A
LC and Subsequent Challenge by BoNT/A Dichain

| | | |
|---|---|---|
| | | Survival at given |
| Dose^{a} | ELISA | BoNT/A dichain challenge^{c} |

| (μg/mouse) | Titer^{b} | 10²LD₅₀ | 10³LD₅₀ |
|---|---|---|---|
| 0^{d} | <100 | 0/5 | 0/5 |
| 5^{d} | 18,000 | 0/10 | 0/10 |
| 15^{d} | 63,100 | 0/10 | 0/10 |
| 0^{e} | <100 | 0/5 | 0/5 |
| 5^{e} | 985 | 0/10 | 0/10 |
| 15^{e} | 2800 | 0/10 | 0/10 |

Although the LC by itself is nontoxic, in digitonin-permeabilized chromaffin cells (Bittner et al., 1989) and direct microinjection into the cytosol of sea urchin eggs (Bi et al., 1995; Steinhardt et al., 1994), it blocks membrane exocytosis. To demonstrate that the rBoNT/A LC preparation retained this property of inhibiting membrane exocytosis, sea urchin eggs were microinjected with the LC. Eggs of the sea urchin, Lytechinus pictus, are an excellent model system for the study of exocytosis. Unfertilized eggs have a layer of vesicles, the cortical granules, docked at the plasma membrane. The SNARE complexes of docked vesicles are inaccessible to the BoNTs. Thus, plasma membrane resealing of the unfertilized sea urchin egg is unaffected by microinjection with botulinum toxins A, B, and C1 (Bi et al., 1995; Steinhardt et al., 1994). Fertilization triggers exocytosis of the cortical granuoles. After fertilization, the vesicles available for exocytosis are largely undocked and the docking proteins of undocked vesicles are susceptible to proteolysis by injected clostridial neurotoxins.

For fertilized eggs injected with rBoNT/A LC, about 100 min at 20° C. was required to inhibit plasma membrane resealing after mechanical wounding with a glass micropipet. Eggs that successfully resealed showed a transient dye loss for about 1-2 min after micropuncture. Eggs that failed to reseal continuously lost dye and lost control of intracellular free calcium, leading to cell death. Five of five fertilized eggs wounded between 36 and 70 min after injection with the rBoNT/A LC resealed successfully, as did five of five unfertilized injected eggs. Six of six fertilized eggs wounded between 106 and 145 min after injection failed to reseal, indicating that the recombinant light chain actively inhibited exocytosis. Thus, the rBoNT/A LC had a similar effect as BoNT/B in inhibiting membrane exocytosis and resealing of plasma membrane of sea urchin eggs (Steinhardt et al., 1994).

### Example 12

Exocytosis Experiments

Plasma membrane resealing after micropuncture with a glass pipette requires calcium-regulated exocytosis (Bi et al., 1995). This exocytosis is dependent on docking proteins (the SNARE complex) that are sensitive to proteolysis by the clostridial neurotoxins (Steinhardt et al., 1994). Sea urchin (Lytechinus pictus) eggs were used to test the biological activity of the rBoNT/A LC. The microinjection medium contained 19 volumes of the rBoNT/A LC (3.7 mg/ml) in 45 mM potassium aspartate, 5 mM HEPES, pH 8.1, and one volume of 55 mM fura-2 in 100 mM KCl and 10 mM HEPES, pH 7.1. Injection levels were 5-10% of egg volume. The plasma membrane resealing after micropuncture with a glass pipette was monitored by recording the emission from fura-2 upon excitation at 358 nm (the calcium-insensitive wave-length).

### Example 13

Other Analytical Methods

Protein concentration was determined by BCA assay (Pierce) with bovine serum albumin (BSA) as a standard. Reducing SDS-PAGE with 10% tricine-gels (Novex) was according to Laemli (1970). The gels were stained with Coomassie brilliant blue. Western blots were prepared by using a primary polyclonal antibody against a 16-residue N-terminal sequence of BoNT/A LC and a peroxidase-coupled goat anti-rabbit IgG (H+ L) as the secondary antibody. Absorption spectrum at 25° C. was recorded in a Hewlett-Packard 8452 diode array spectrophotometer. The N-terminal amino acid sequence of the BONT/A LC was determined by Edman degradation in an Applied Biosystems Procise Sequencer in the 0- to 20-pmol detection range. Molecular mass was determined by MALDI-MS in a PE Biosystems Voyager DE instrument. Sinapinic acid was used as the matrix and the sample was spotted on a stainless steel plate that was not washed with water or TFA. Other conditions in the experiment were accelerating voltage 25,000 V, guide wire voltage 0.3%, and laser 2500.

### Example 14

Chemicals, Buffers and Reagents

Buffer P (50 mM Na-phosphate, pH 6.5) was used for Examples 14-20. TPEN and ZnCl₂ were from Sigma. Affinity-purified, peroxidase-coupled goat anti-rabbit and anti-mouse IgG (H+ L) and ABTS substrate were from Kirkegaard Perry Laboratories (Gaithersburg, Md.). The inhibitor peptide (Ac-CRATKML-NH₂) (SEQ ID NO: 46) (Schmidt et al., 1998) was synthesized and purified by Cell Essentials (Boston, Mass.).

### Example 15

BoNT/A LC Purification

The rBoNT/A LC was expressed by low-temperature IPTG induction in E. coli BL21 (DE3) cells as a soluble protein from a synthetic gene in a pET24a-derived multicopy plasmid (Clontech, Inc.). Construction of the gene and expression of the protein as described (Ahmed and Smith, 2000) was modified as follows: a stop codon replaced the histidine tag at the carboxy terminus of the gene, and induction and expression was at 18° C. for 22-24 hr. The LC was purified to near homogeneity by NaCl gradient elution from each of two successive cation exchange columns (MonoS) in buffer P. A typical preparation had a specific activity of 2-3:mol/min/mg in cleaving the 17-residue substrate peptide when assayed in the presence of 0.25 mM ZnCl₂; in the absence of added zinc, activity was 50%. The purified LC was thus partially resolved of the bound zinc. The purified protein (1-4 ml) in buffer P was stored at −20° C. Under this condition, the protein remains stable and retains its catalytic activity for at least 1 year.

### Example 16

SDS-PAGE, Transfer on PVDF Membrane, and Western Blot

SDS-PAGE under reducing conditions (Laemmli, 1970) was carried out on a 1-mm-thick 10% tricine gels (Novex) as described (Schagger and von Jagow, 1987). Samples were prepared in 0.4% SDS, 5% ∃-mercaptoethanol, 12% glycerol, and 450 mM Tris-HCl, ph 8.45, by boiling for 5 min. The running buffer contained 0.1% SDS in 0.1 M Tris-0.1 M Tricine, ph 8.3. The gels were stained with Coomassie Brilliant Blue. Electrophoretic transfer of peptides from SDS-PAGE gels onto PVDF membrane used 10 mM CAPS-NaOH buffer, Ph 11.0, containing 10% methanol as the transfer buffer. Protein bands on the PVDF membranes were visualized by 1 min of staining with Coomassie Brilliant Blue followed by destaining in 10% acetic acid-5% methanol. The stained bands were cut out from the dried membranes for amino-terminal sequence determination. Western blots on nitro-cellulose membranes were prepared using a primary polyclonal antibody against a 16-residue N-terminal sequence of BoNT/A LC and a peroxidase-coupled goat anti-rabbit IgG (H+ L) as the secondary antibody (Ahmed and Smith, 2000).

### Example 17

Proteolysis Experiments

Before each experiment, aliquots of the protein were thawed to room temperature and were immediately passed through a PD-10 column to remove the EDTA. The protein was collected in buffer P and stored on ice. The EDTA-free BoNT/A LC was mixed with predetermined concentrations of ZnCl₂, EDTA, TPEN, or the inhibitor peptide and 20-50:1 was distributed in screw-capped Eppendorf tubes. The tubes were incubated at 4° C. or at 22° C. The final concentration of the protein was 0.18-0.20 mg/ml in these incubation mixtures. At various time intervals an equal volume (20-50:1) of SDS-load buffer was added to a tube for SDS-PAGE analysis.

A 100 mM stock solution of TPEN was prepared in ethanol (95%). Stock solutions of the competitive inhibitor peptide Ac-CRATKML-NH₂ (SEQ ID NO: 46) (Schmidt et al., 1998) (5 mM), ZnCl₂ (1-4 mM), and EDTA (20 mM) were prepared in buffer P. Unless otherwise mentioned, final concentrations of these reagents in the incubation mixtures with the LC were TPEN 5 mM, EDTA 5 mM, peptide 1 mM, and ZnCl₂ 0.25 mM.

Results: Cleavage and Fragmentation of BoNT/A LC

FIG. 10 shows that the BoNT/A LC undergoes cleavage and fragmentation that increases with time. The intensity of the band representing the full-length LC with a polypeptide mass of ˜52 kDa (IA) gradually diminished with time and a new protein band of ˜50 kDa (IB) appeared in its place. The results suggest truncation of about 2 kDa mass from the full-length LC. In Western blots (FIG. 10B), both IA and IB also reacted with a rabbit polyclonal antibody raised against a 16-residue amino-terminal sequence of LC. This result suggests that the truncation from the full-length LC must occur at the C-terminus. Indeed, amino-terminal sequencing of the isolated, truncated protein showed the amino terminus was intact. Interestingly, preservation of the N-terminus of full-length BoNT/A neurotoxin was also observed after its posttranslation modification in bacterial culture (DasGupta and Dekleva, 1990). As the truncated protein IB accumulated, a protein band of ˜100 kDa (II) appeared that was detected easily in the Western blot (FIG. 10B). FIG. 10 also shows that at 2 weeks of incubation, the LC fragmented into IIIA+IIIB and IVC. The larger fragment (IIIA) above the 34-kDa marker was followed by a fainter fragment (IIIB) just below the 34-kDa marker. The results of this time course experiment also suggested that IIIB was formed from IIIA. Both of these fragments must represent the N-terminus of the LC, as they reacted with the antibody (FIG. 10B). On the other hand, a much smaller fragment (IVC) moving faster than the 23-kDa marker was probably the C-terminal fragment, as it failed to react with the antibody (specific for the N-terminus of the LC) in the Western blot. The truncation and fragmentation shown in FIG. 10 were independent of the batch of E. Coli cell culture or the batch of purification of the LC.

Results: Zinc Accelerates the Truncation and Fragmentation

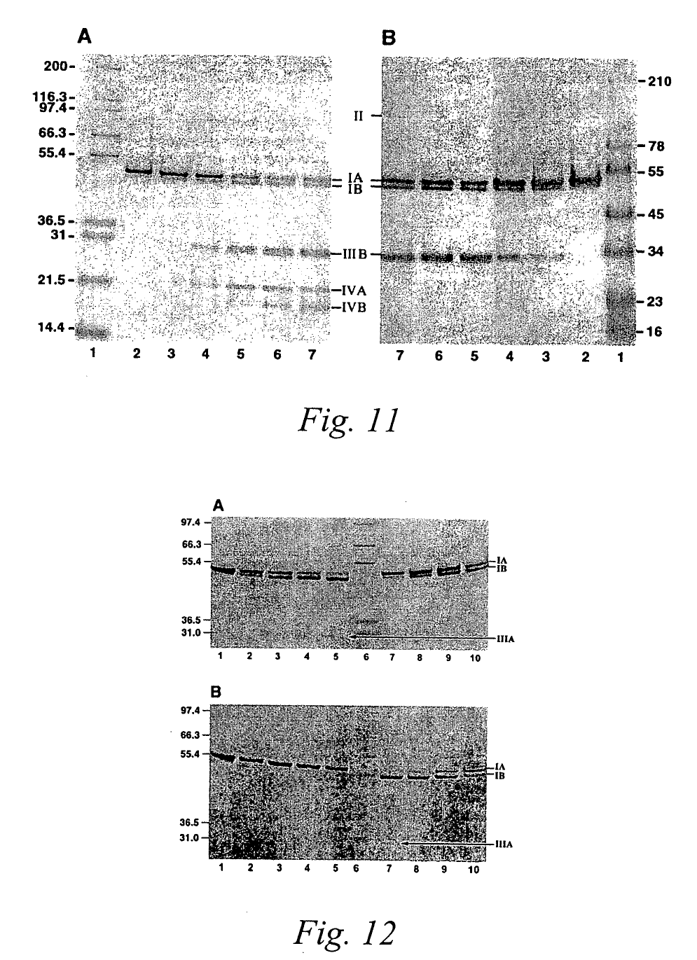

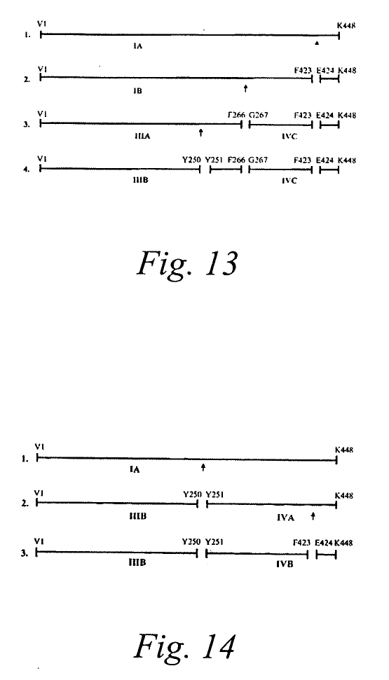

The BoNT/A LC is known to be highly substrate specific. Therefore, the truncation of about 2 kDa from the C-terminus or fragmentation into larger fragments upon storage of the LC at 4° C. described in FIG. 10 might appear to be due to the presence of some contaminating protease in the LC preparation. However, no additional Coomassie-stained protein bands were detected when 0.4-4.0:g of the LC was electrophoresed in the presence of SDS. BoNT/A LC is a zinc-endopeptidase. FIG. 11 shows that when LC was incubated with 0.25 mM ZnCl₂, the rate of fragmentation was greatly increased so that the antibody-reacting fragment IIIB and an antibody-nonreacting fragment IVA appeared within 2 days of incubation (FIG. 11A, B). Fragment IVB appeared later in the time course. Qualitatively, the results are similar to those in FIG. 10 except that in the presence of ZnCl₂, the rate of fragmentation was higher, fragment IIIB was formed without showing the initial formation of IIIA, and initial formation of IVA gave rise to IVB. The rate enhancement by zinc could be partly due to formation of holo-LC from the partially Zn-resolved LC (see Section 2). Because there was no fragment IVC (FIG. 10) detected in this experiment (FIG. 11), zinc must also have a structural role in the LC. From the results shown in FIG. 11A it is not possible to judge if the C-terminal truncation of IA in forming IB and dimerization in forming II precede the fragmentation into III and IV. However, in some other experiments, using a lower concentration of ZnCl₂, it was possible to show that formation of IIIB occurred before formation of IB and that fragmentation was the last event.

The rates of C-terminal truncation and fragmentation of LC either in the absence or in the presence of ZnCl₂ were much higher when incubated at 22° C. than at 4° C. In fact, amino-terminal sequence was determined on the fragments generated by incubation at 22° C. for 2 days only.

Results: Metal Chelator TPEN Inhibits Truncation and Fragmentation

As shown in FIG. 11, if the C-terminal truncation and fragmentation of the LC was indeed dependent on the presence of zinc, removing zinc from the incubation mixture and from the active site of the LC would be expected to abolish the truncation and fragmentation events. However, zinc is very tightly bound to the active site of LC. Extensive treatment with 10 mM EDTA in the cold (Ahmed and Smith, 2000) or with 10 mM EDTA at room temperature (Li and Singh, 2000) failed to completely remove zinc from the active site of the LC. In agreement with these observations, including 10 mM EDTA failed to protect the LC from C-terminal truncation and processing (FIG. 12A). In contrast, the metal chelator TPEN largely protected the LC from truncation and fragmentation (FIG. 12A). It was also found that, at 1 mM TPEN, the LC showed no activity when assayed for 5 min. Because the incubation mixture with TPEN did not contain any exogenous metal or zinc, any chelation by TPEN must have involved the active-site zinc of the LC. These results also suggest that truncation and fragmentation of the LC upon storage 4° C. or at room temperature were autocatalytic.

### Example 18

Separation of Peptides with HPLC and their Characterization by ESIMS-MS

For mass and sequence determination, peptides were separated on an Agilent Technologies Series 1100 liquid chromatograph with a 0.8×100 mm Poros-2 R/H column (PerSeptive Biosystems, Inc.). The mobile phase was 0.1% formic acid (solvent A) and 80% acetonitrile in 0.1% formic acid (solvent B). The peptides were eluted with a linear gradient of 0-100% B over 15 min at a flow rate of 0.2 ml/min. The injection volume was 10:1. The peptides were detected and structurally characterized on a Finnigan LCQ Deca mass spectrometer employing data-dependent MS/MS. Molecular mass was also determined by MALDI-MS with a PE Biosystems Voyager DE instrument. Sinapinic acid was used as the matrix, and the sample was spotted on a stainless steel plate that was not washed with water or TFA. Other conditions in the experiment were accelerating voltage 25,000 V, guide wire voltage 0.3%, and laser 2500.

Results: Amino Acid Sequence of the Small Peptides Generated by C-Terminal Processing

To map the sites of proteolysis, the small peptides were isolated by ultrafiltration of a C-terminally truncated LC mixture. Amino acid sequences of these peptides were determined by ESIMS-MS (Table 4). The peptides with G433 at the amino terminus (peptide 4) and K438 at the carboxy terminus (peptide 5) indicated cleavage by a trypsin-like protease on the R432-G433 and K438-T439 bonds, respectively. Of these, only the lysyl bond at K438 was reported to be cleaved by a clostridial endogenous protease or by trypsin (DasGupta and Dekleva, 1990). However, a cleavage at the K444-G445 bond as reported before by an endogenous clostridial protease (DasGupta and Dekleva, 1990) was not detected. Neither was cleavage detected at K440-S441 or at K427-L428 bonds, the other potential sites of tryptic cleavage. Although these results indicated that the LC preparations did not contain a protease activity that could cleave at K427-L428, K440-S441, and K444-G445, it is equally possible that some of the small peptides generated by cleavage at these sites were lost during sample preparation. Interesting findings of this experiment (Table 4) are the peptides with N-terminus of T420 (peptide 1) and V431 (peptide 3), as the preceding residues at F419-T420 and C430-V431 bonds, respectively, are certainly not the sites of “tryptic” cleavage.

TABLE 4

C-Terminal Peptides Generated after Initial
Cleavage of the BoNT/A LC^{a}

| | | 420 425 430 435 440 445 |
|---|---|---|
| | | \| \| \| \| \| \| |
| Peptide | Mass^{b} | KNFTGLFEFYKLLCVRGIITSKTKSLDKGYNK^{c} |
| 1 | 2188 (2188) | TGLFEFYKLLCVRGIITSK |
| 2 | 2124 (2112)^{d} | CVRGIITSKTKSLDKGYNK^{d} |
| 3 | 2008 (2008) | VRGIITSKTKSLDKGYNK |
| 4 | 1753 (1753) | GIITSKTKSLDKGYNK |
| 5 | 989 (977)^{d} | CVRGIITSK^{d} |

^{a}The peptides were generated by incubating 0.4 mg of the LC in 0.5 ml of buffer P at 4° C. for 2 weeks. They were isolated by ultrafiltration through a Centricon CM10 (Amicon) membrane that was previously treated with 10 mM EDTA. The filtrate containing the peptides was stored at −20° C. for 1 week before mass and sequence determinations by ESIMS-MS. The sequence on the first row with the numbers above it represents the known C-terminal sequence of the LC (Ahmed and Smith, 2000).
^{b}Experimentally determined mass from ESI-MS; calculated mass for the sequence shown is given in parentheses.
^{c}Residues 417-448 of SEQ ID NO:5
^{d}The calculated mass was 12.1 Da smaller than the experimental value. Except for cysteine in peptides 2 and 5, the experimentally determined masses of all other amino acid residues agree well with their calculated values. Note that cysteine in peptides 2 and 5 occurred at the N-terminus, but when it was in the middle of the peptide, there was no ambiguity in the results.

The sequence data from the ESIMS-MS results for the peptides 2 and 5 agree very well with the residue stretches V432-K449 and with the residue stretches V432-K449 and with the residue stretches V431-K438, respectively. However the experimentally determined mass for “C430,” the residue at the amino side of V431 in both peptides, was greater by 12.1 Dalton than the theoretical mass for cysteine. At this stage, there is some uncertainty regarding the discrepancy in the mass of this “cysteine.” Chemical modification experiments using iodoacetamide or acidified methanol failed to shift the masses of these peptides, indicating that the suspected “cysteine” did not have a free sulfhydryl group nor was a contaminating aspartic acid. Cysteine in proteins are known to occur as derivatives such as cysteine sulfenic acids (Ahmed and Claiborne, 1992; Claiborne et al., 1999). Attempts are being made to decipher the chemical nature of this “cysteine.” If indeed it was a modified C430, cleavages at the carboxy ends of F419, C430, and V431 in addition to R432, K438, and K438 indicate that the proteolytic activity in this preparation was not “tryptic” in nature, but had a broad specificity.

Results: Identity of the Large Peptides Generated by Fragmentation

The large peptides generated by fragmentation in the middle of the LC were identified by comparing the mass determined by MS with a calculated mass for a stretch of sequence based on the amino-terminal sequence determination (Table 5). Agreements between the experimental and calculated values were within 0.07%. Identity of IIIA as having a sequence range of V1-F266 was based on the kinetics of its (and of IVC's) appearance on SDS-PAGE (FIGS. 10 and 11) and N-terminal sequence of IVC. The sequence data along with Western blot results clearly demonstrated that the amino terminus of the LC (IA and IB) remained unchanged during the prolonged incubation period. Although the C-terminal sequence of the peptides IIIA and IIIB was not determined, N-terminal sequences of the peptides IVA, IVB, and IVC (Table 5) indicate that fragmentation of IA and IB (FIGS. 10 and 11) occurred by cleavage at the Y250-Y251 and F266-G267 bonds. Again, if the cleavages of these tyrosyl and phenylalanyl bonds were catalyzed by a protease, it must have been “nontryptic” in nature. Identity of the peptides IVB and IVC as having F423 at the C-terminal indicated that a C-terminal processing of the LC at F423-E424 remained undetected in the small peptide isolation experiment (see previous section). This result nonetheless supports that C-terminal processing occurred at phenylalanyl bonds in addition to lysyl, arginyl, valyl, and (most likely) cysteinyl bonds.

TABLE 5

Identity of the Polypeptides Generated
by Proteolysis of the BoNT/A LC

| | Mass | Mass | Sequence | N-terminal |
|---|---|---|---|---|
| Peptide^{a} | (Exp) | (Calc) | range | sequence |
| IA | 51,315 | 51,318 | V1-K448 | 2-VQFVNKQ |
| IB | 48,866 | 48,870 | V1-Y426 | 2-VQFVNKQ |
| II | 97,727^{b} | | | |
| | 97,870^{b} | | | |
| IIIA | n.d.^{c} | 32,270 | V1-F266 | 2-VQFVNKQ |
| IIIB | 28,111 | 28,130 | V1-Y251 | 2-VQFVNKQ |
| IVA | 23,207 | 23,207 | Y252-K448 | 252-YEMSGLE |
| IVB | 20,319 | 20,319 | Y252-F423 | 252-YEMSGLE |
| IVC | 18,400 | 18,400 | G267-F423 | 267-GGHDAKF |
| ^{a}Peptide designations are from FIGS. 10 and 11. Mass was determined by ESIMS-MS. Masses of the peptides IA and IB were determined separately. Peptides were generated by incubating the LC (1.8 mg/ml buffer P) alone or in the presence of 0.25 mM ZnCl₂ for 2 days at 22° C. Partial precipitation of the protein was visible after 1 day and was removed by centrifugation before ESI analysis. Masses of IIIB, IVA, and IVB were determined in samples containing ZnCl₂ and those | | | | |
| of IA, IB, IIIA, and IVC were determined in samples with no ZnCl₂. Calculated masses are for the sequence ranges shown based on N-terminal sequence and mass data. The N-terminal sequences were determined separately for IA (residues 2 to 8 of SEQ ID NO:5), IB (residues 2 to 8 of SEQ ID NO:5), and IIIA (residues 2 to 8 of SEQ ID NO:5) in solutions and for IIIB (residues 2 to 8 of SEQ ID NO:5), IVA (residues 252 to 258 of SEQ ID NO:5), IVB (residues 252 to 258 of SEQ ID NO:5) and IVC (residues 267 | | | | |
| to 274 of SEQ ID NO:5) on PVDF membrane after separation by SDS-PAGE and transfer on membrane. | | | | |
| ^{b}Data from MALDI-MS determined in a sample containing IB with an initial concentration of 0.2 mg/ml. | | | | |
| ^{c}Mass could not be detected in several experiments, probably due either to precipitation or to irreversible binding to column resin. Although a peptide with a lower mass can have slower mobility than a homologous higher mass peptide in SDS-PAGE due to charge differences (Ahmed et al., 1986), the kinetics of appearance of IIIB from IIIA (FIG. 1) and their identification by N-terminal sequence determination suggest that IIIA must be larger than IIIB. Identity of IIIA as having a sequence of | | | | |
| V1-F266 with a mass of 32,270 was based on N-terminal amino acid sequence determination and SDS-PAGE results (FIGS. 10 and 11). | | | | |

### Example 19

Other Analytical Methods

The enzymatic assay was based on HPLC separation and measurement of the nicked products from a 17-residue C-terminal peptide of SNAP-25 corresponding to residues 187-203 (Schmidt and Bostian, 1995). Initially protein concentrations were determined by BCA assay (Pierce) with bovine serum albumin (BSA) as a standard. After it was established by repeated measurements that a 1-mg/ml BoNT/A LC thus determined has A^{0.1%} (1 cm light path) value of 1.0 at 278 nm (0.98 at 280 nm), protein concentration was determined from absorbance at 278 nm. For comparison, the calculated A^{0.1%} value of the LC at 280 nm in water (Pace et al., 1995) is 0.948. Absorption spectra were recorded in a Hewlett-Packard 8452 diode array spectrophotometer. The N-terminal amino acid sequence of the LC was determined by Edman degradation in the Applied Biosystems Procise Sequences in the 0- to 20-pmol detection range.

### Example 20

A Specific Competitive Inhibitor of LC Activity was an Effective Inhibitor of Truncation and Fragmentation

Autocatalytic truncation and fragmentation of proteins can arise from chemical catalysis and from enzymatic catalysis. To differentiate these two possibilities, a peptide specifically synthesized as a competitive inhibitor of BoNT/A proteolytic activity (Schmidt et al., 1998) was used. This peptide inhibitor, with a sequence of CRATKML (SEQ ID. NO:46), competitively inhibits the cleavage of a 17-residue substrate peptide based on SNAP-25 by BoNT/A neurotoxin with a Kᵢ of 2 uM (Schmidt et al., 1998). At a 1 mM inhibitor peptide concentration, the LC showed no activity when assayed for 5 min. FIG. 12B shows that when the LC was incubated with 1 mM peptide inhibitor, both C-terminal truncation and fragmentation at the interior of LC were largely prevented. In the presence of the peptide inhibitor, however, the LC underwent a very slow cleavage, as can be expected in an enzymatic activity with a competitive inhibitor. Densitometric scanning of the gel showed that after 28 days, in the presence of the peptide inhibitor, less than 10% of the LC (IA) was converted into the C-terminally truncated form (IB). In contrast, in the absence of the peptide inhibitor, more than 80% of the LC (IA) was converted into the truncated form (IB). Results of this experiment prove that loss of 10-28 residues from the C-terminus of LC followed by fragmentation into two major peptides (FIGS. 10 and 11, Tables 4 and 5) occurred at the active site of the LC and that these reactions were enzymatic. The results also provide direct evidence that the cleavage reactions were not due to any contaminating protease in the preparation of the LC.

### Example 21

Materials

PCR-TOPO and 1-Shot cells were from Invitrogen. pET24a plasmid and BL21 (DE3) cells were obtained from Novagen. All were prepared by standard methods. Proteins were visualized by SDS-PAGE and stained either with Coomassie or Colloidal Coomassie (Novex). Westerns (Novex) were reacted with a rabbit primary antibody (Research Genetics, Inc., Huntsville, Ala.) against the N-terminal 16 amino acids (PFVNKQFNYKDPVNGV; SEQ ID NO:1) of the LC of type A and were visualized with a horseradish peroxidase conjugated goat anti-rabbit secondary anti-body and TMB peroxidase substrate (Kirkegaard Perry Laboratories). Bacterial media was from Difco. Purification of the expressed proteins was on a Pharmacia model 500 FPLC system with programmed elution and A₂₈₀ monitoring (Pharmacia, Uppsala, Sweden). Columns were a Pharmacia HR 10/10 Mono S cation-exchange column, a Pharmacia Mono S 5/5 cation exchange column, and a Perseptive Biosystems POROS 20 HS cation exchange column. Pretreatment of the expressed proteins was with DNase (Sigma, Inc.) and dialysis was with Pierce Slide-A-Lyzer 10 k MWCO cassettes. The SNAP-25 substrate peptide (Quality Controlled Biochemicals, Hopkinton, Mass.) and its cleavage products were separated on a Hi-Pore C 18 column, 0.45×25 cm (Bio-Rad Laboratories) and analyzed with the Millennium Software Package (Waters, Inc.). Src (p60c-src) recombinant phosphokinase, substrate peptide, and anti-phosphotyrosine monoclonal antibody 4G10 were from Upstate Biotechnology, Lake Placid, N.Y. [γ-³²P]ATP, 3000 Ci/mmol, was from Dupont-NEN.

### Example 22

Preparation of Recombinant Neurotoxin Clones

New restriction sites were added by PCR to the 5′ and 3′ ends (NdeI and HindIII, respectively) of the synthetic DNA molecules coding for the Lc (M₁, to K₄₄₉), the Lc plus belt (LC+Belt; M₁, to F₅₅₀) and the Lc plus translocation region (LC+Xloc; M₁ to Q₆₅₉). These sequences correspond to GenBank accession numbers x, y and z respectively. PCR products were subcloned into pCR-TOPO and the sequences confirmed by DNA sequencing. The inserts were cut from the subcloning vector and ligated behind the NdeI site of pET24a, so as to begin expression with the initial methionine of the LC. The plasmid was transformed into E. coli BL21 (DE3) cells for expression.

### Example 23

Expression of Neurotoxins

One hundred ml of Terrific Broth (TB) plus kanamycin was inoculated with the appropriate clone and grown overnight, with shaking, at 37° C. Fifty ml of LcA or 100 ml LcA+Belt and Lc+Hn of overnight growth was added to 1 liter TB plus kanamycin and shaking incubation continued at 37° C. for an additional 1.25 hours. While cultures were placed on ice for 5 to 10 minutes, the OD₆₀₀ was read and adjusted to approximately 0.4 to 0.6, then IPTG was added to 1 mM for induction of protein expression. Duplicate cultures were grown at 37° C. (4 hours), 30° C. (10 hours) and 18° C. (22 hours). At harvesting, the OD₆₀₀ was read again, cells were pelleted and frozen at −70° C. if not used immediately. Data points are the mean of three separate measurements of the appropriate bands from SDS-PAGE gels scanned and digitally analyzed with an AlphaImager 2000 densitometer and AlphaImager Documentation and Analysis Software (AlphaInotech, San Leandro, Calif.).

Expression at Low Temperatures Markedly Increases Yields of Soluble Product, While Addition of Portions of the Hn does not Increase the Yield of Soluble Product

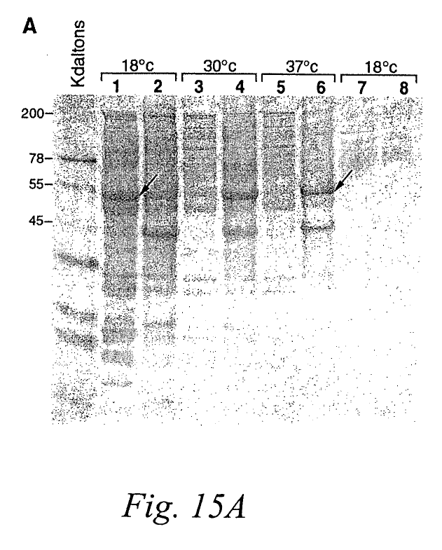

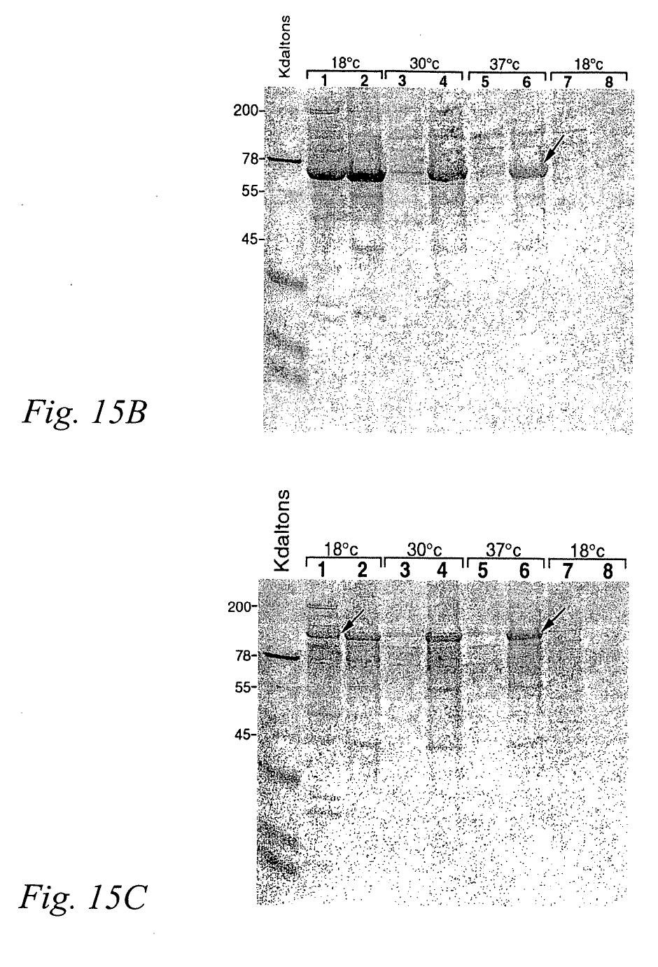

To study the effects of low temperature induction on the expression of LcA, expression was performed at 18° C., 30° C. and 37° C. FIG. 15A shows the decreasing solubility of LcA at these three temperatures, with concomitant decrease in the soluble product, from 55.5% at 18° C. to 5.2% at 37° C. Yields of soluble LcA were highest at 18° C., with LcA making up approximately 10% of the cell protein. Addition of the belt and Hn portions of the neurotoxin to LcA did not increase solubility (FIGS. 15A, 15B and 15C), although addition of the full Hn region reduced expression and yield (FIG. 15C).

Constructs were grown both in Luria Broth (LB) and Terrific Broth (TB), with no apparent difference in the quality or percent solubility of the products. Total yield was far greater for growth in TB, 17.97 g/l verses 7.77 g/l for LB. Optimal expression conditions for the Lc were considered to be the construct lacking either the belt or the Hn region at 18° C. for 20-24 hours in TB.

### Example 24

Sample Preparation and Purification of LC

One gram E. coli cell paste was resuspended into 20 ml of buffer A (20 mM NaAcetate, 2 mM EDTA, pH5.4). The suspended cells were disrupted by sonicating for 12 cycles of 30 seconds followed by 30 seconds of incubation on ice using a medium size probe at 65% output. The resulting cell lysate was centrifuged (Sorval) at 15,000×g for 15 minutes at 4° C. to separate the proteins into soluble and insoluble fractions. The soluble fraction was diluted 1:1 in equilibration buffer B (20 mM NaAcetate, 2 mM EDTA, pH5.8) and used as starting material for the chromatography.

A HR 10/10 Mono S cation-exchange column was extensively cleaned between runs by sequentially running through it: 1 M NaCl through at 3 ml/min for 5 minutes; 20 mM NaOH for 10 minutes at 1 ml/min; 70% ethanol in ddwater for 30 minutes at 1 ml/min; 1 M NaCl in buffer B for 15 minutes at 1 ml/min; then re-equilibrated with buffer B at 2 ml/min for 5 minutes. The diluted lysate was then loaded at a flow rate of 2 ml/min (150 cm/h). The column was washed with 24 ml (3 bed volumes) of buffer B. Flow through and wash were collected separately and stored for subsequent analysis. Protein was eluted from the column with a linear gradient from 0 to 70% 1 M NaCl in buffer B over 8 minutes. Two-ml fractions were collected throughout the gradient. Fractions eluting between 10 and 22 mSiemanns (mS) were positive for rBoNTA(L_{C}) as shown by Western blot analysis. The pooled fractions were diluted 1:3 with buffer C (20 mM NaAcetate, 2 mM EDTA, pH6.2) and loaded onto a Mono S 5/5 cation exchange column equilibrated with buffer C at a flow rate of 2.5 ml/min. The column was washed with 10 ml (10 bed volume) of buffer C. Protein was eluted from the column with a linear gradient of 0-75% 1 M NaCl in buffer C over 15 minutes. The rBoNTA(L_{C}) protein eluted from the Mono S column as a single band at 12 mS as shown by Western blot analysis. Fractions were pooled and stored frozen at −20° C. in plastic vials. The product was greater than 98% pure as determined by SDS-PAGE.

The LcA+Belt and the LcA+Hn were similarly purified, except that sonication was in buffer A (20 mM NaAcetate, 2 mM EDTA buffer, pH 4.8) and dilution was not necessary after centifugation to obtain the soluble fraction. After extensive cleaning of the column, the soluble fractions of either LcA+Belt or LcA+Hn were loaded at 2 ml/min onto a Poros 20 HS column equilibrated with buffer A. After loading, the column was rinsed at 3 ml/min with buffer A for 5 minutes and a 5% step of 1 M NaCl in buffer A was performed to remove interfering cellular products. The LcA+Belt was then eluted with a 9% step and the LcA+Hn eluted with a 10-14% step of 1 M NaCl in buffer A. Fractions were pooled, diluted 1:3 with equilibration buffer A and re-run on the HS column, eluting with a 1 to 75% gradient of 1 M NaCl in buffer A. Verification of the peaks was by Western blot and SDS-PAGE. Each protein was 95% or greater pure. Fractions were pooled and stored frozen at −20° C. in plastic vials.

After the first column purification, aliquots of the expressed LcA+Hn were additionally nicked with trypsin at 10 μg/ml overnight, at room temperature. This semi-purified protein lysate was then diluted and run on a second Poros HS column as described above. Protein was similarly 95% or greater pure.

Total protein concentrations were determined by using either a Bio-Rad Protein assay at one-half volume of the standard protocol and bovine serum albumen as the protein standard or the Pierce BCA (bicinchoninic acid) protein assay with the microscale protocol as directed, with bovine serum albumin as the protein standard.

Purification of the Lc from the Soluble Fraction of the Lowest Temperature Expressed

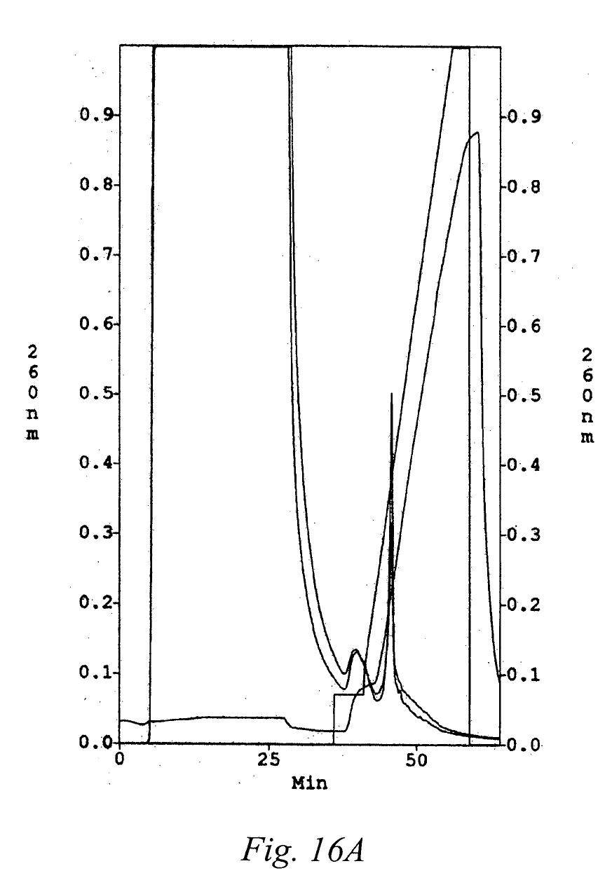

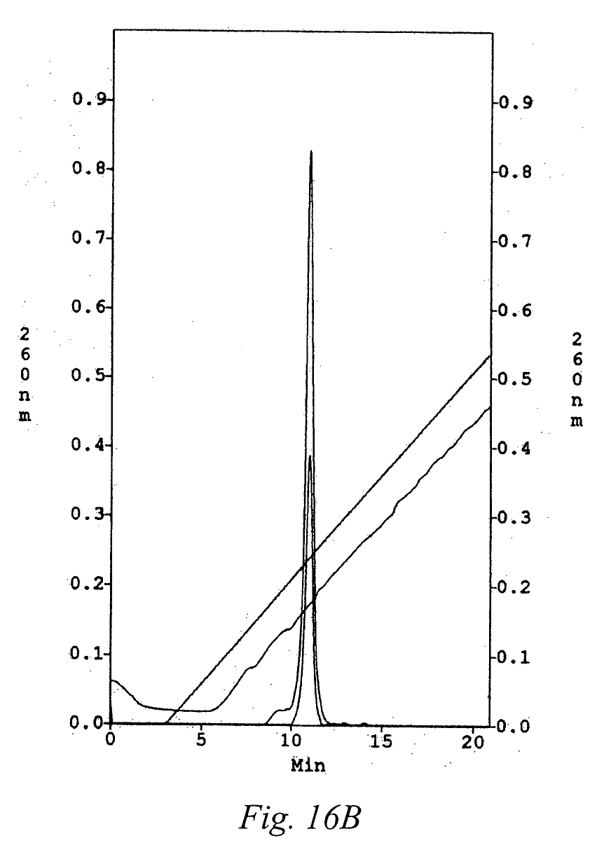

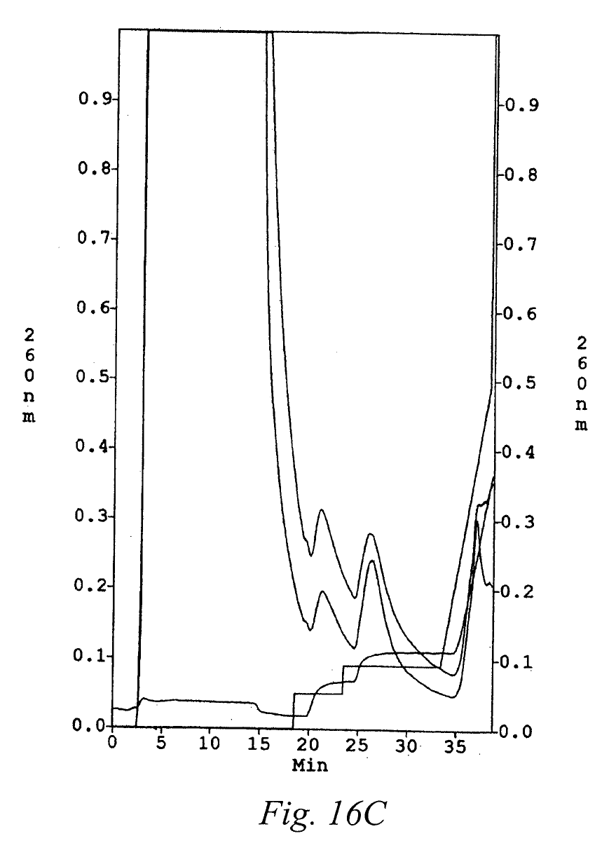

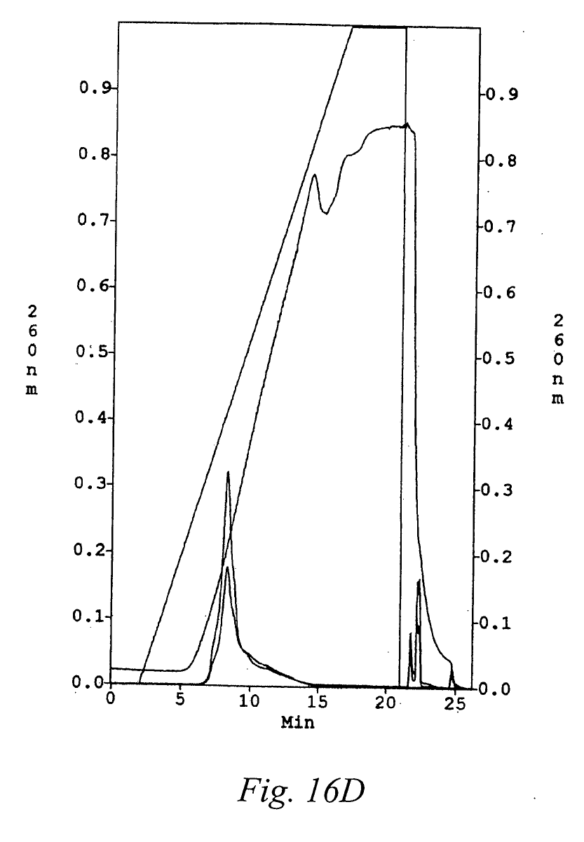

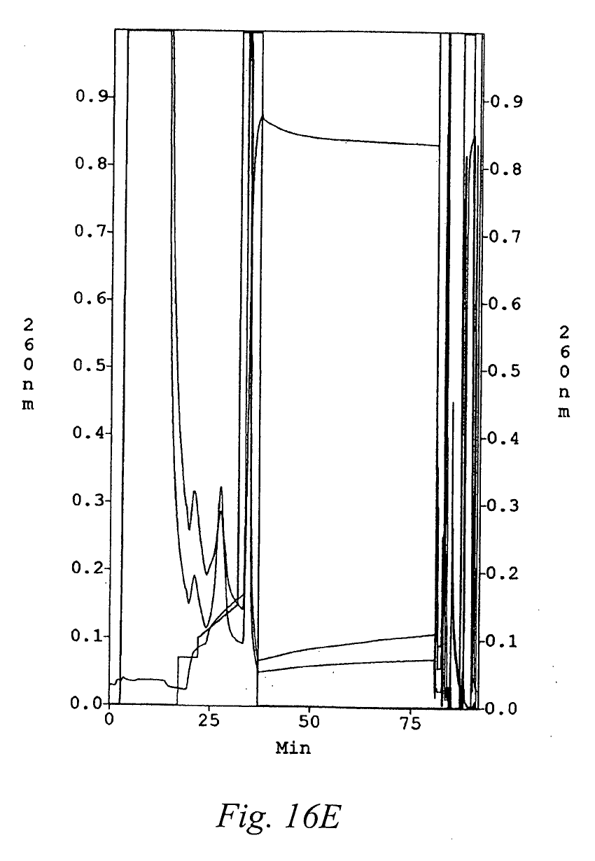

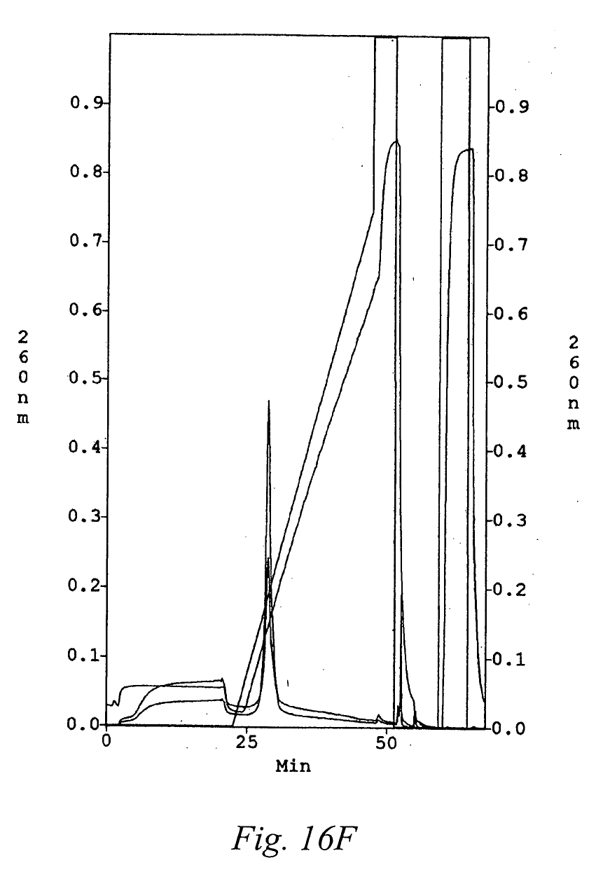

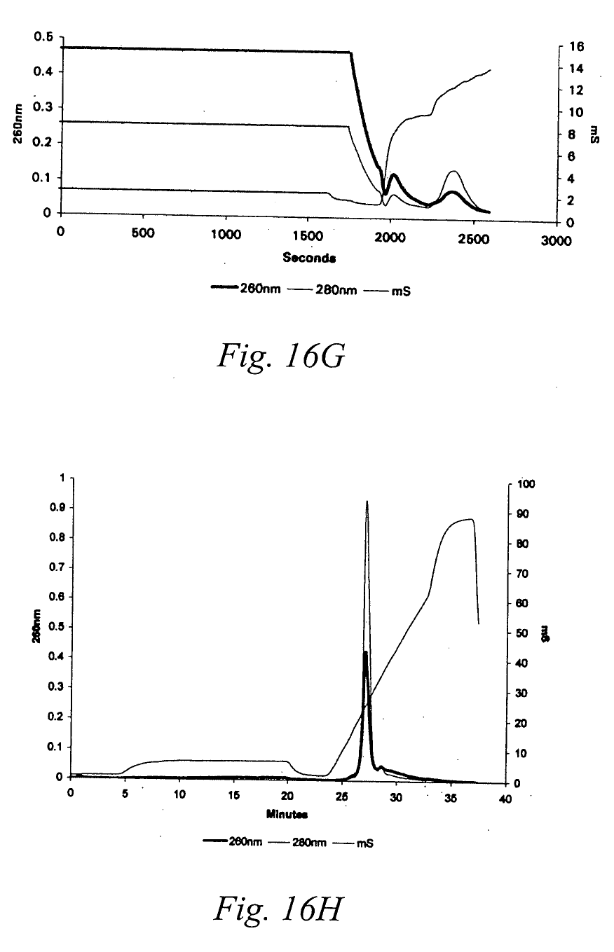

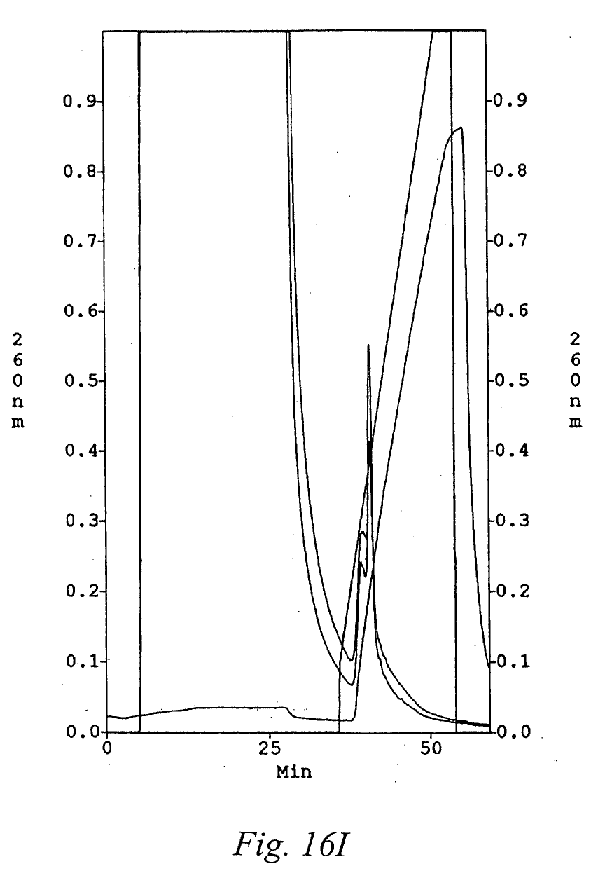

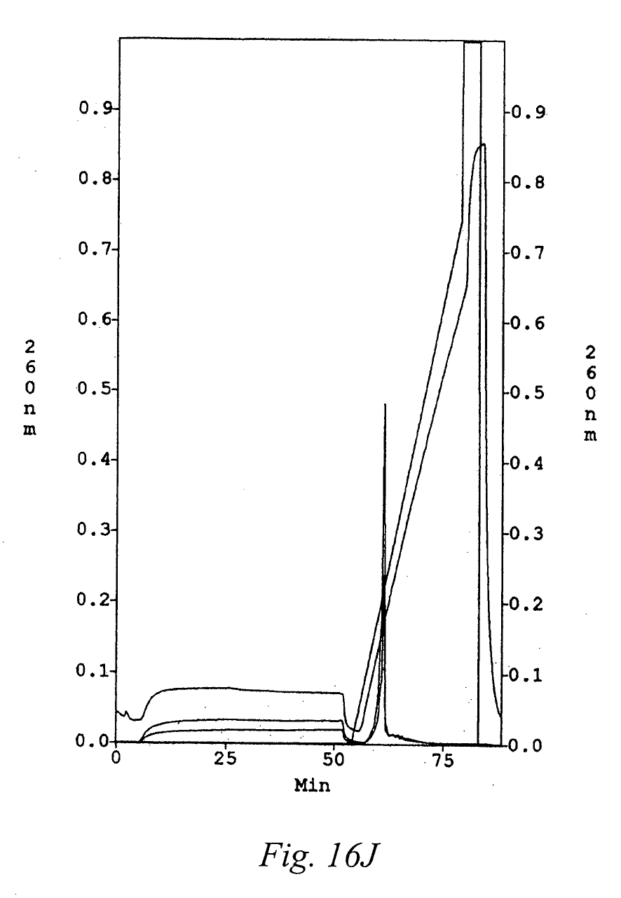

Once conditions had been achieved for optimal yield of product, recovery of the Lc by simple cell sonication was deemed sufficient to release the protein. After removal of insoluble cell debris and proteins by centrifugation, this extract was directly loaded onto a cation exchange column and two isoforms of the Lc were observed to elute between 180 and 280 mM NaCl (FIG. 16A). Western analysis of collected fractions showed two peaks reactive to antisera, corresponding to a full length Lc, and a Lc truncated by approximately 2.5 kDa. Since both forms were reactive to the amino terminus specific sera, a carboxy terminus truncation was indicated. The calculated pI for a Lc lacking the terminal 21 residues is 6.39, suggesting that it would be eluted at a lower NaCl concentration, as was observed. This difference in elution conditions allowed for a separate purification of each Lc isoform. The products eluted from the cation exchange chromatography column were observed to be approximately 70% pure, with a total protein concentration of 1.1 mg/ml.

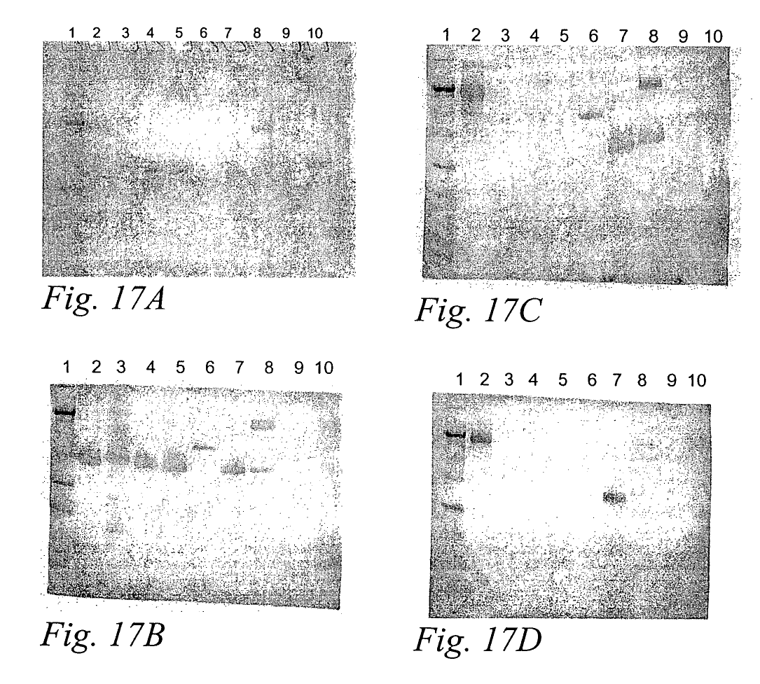

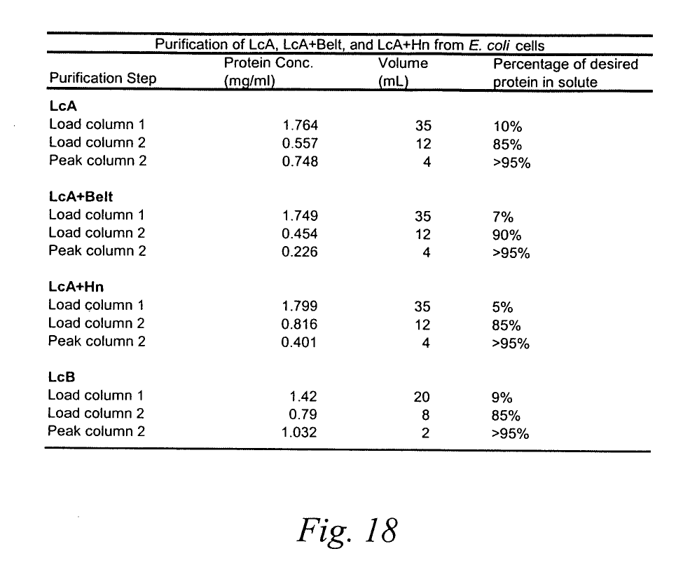

The material was reloaded onto the Mono S column for further purification. The larger, non-truncated, LcA eluted as a single peak at 12 mS (FIG. 16B). SDS-Page and western blot analysis showed only a single band at 51 k-Da (FIGS. 17A and 17B). The product was judged to be 98% pure after the final step and a protein determination determined the overall yield was 0.53 mg purified Lc per gram wet cells obtainable from our protocol.

The LcA+Belt eluted from the first column purification was approximately 85% pure, with a protein concentration of 0.454 mg/ml, in a total of 12 ml (FIG. 2C). After purification on the second column, a 4 ml pooled peak (FIG. 16D) had a concentration of 0.226 mg/ml, with 98% purity, producing a single band as observed by Western analysis (FIGS. 17A and 17C). The overall yield was 0.347 mg/gm wet cells.

The LcA+Hn eluted from the first column purification was approximately 80% pure, with a protein concentration of 0.816 mg/ml, in a total of 12 ml (FIG. 16D). After purification on the second column, a 4 ml pooled peak (FIG. 16E) had a concentration of 0.401 mg/ml, with 98% purity, forming a single band, while the nicked form of the construct produced two bands (FIGS. 17A through 17D) corresponding to the Hn and Lc. The overall yield was 0.617 mg/gm wet cells.

### Example 25

Assay for Cleavage of SNAP-25 Peptide

A 17-residue C-terminal peptide of SNAP-25 (acetyl-SNKTRIDEANQRATKML-amide) (SEQ ID NO:2) shown to be the minimum length required for optimal BoNT/A proteolytic activity (Schmidt and Bostian, 1997) was used as the substrate in a cleavage assay as described previously (Ashraf Ahmed et al.). Briefly, a 0.3 ml mixture containing 0.7-1.0 mM of the substrate peptide, 0.25 mM ZnCl₂, 5.0 mM DTT, 50 mM Na-HEPES buffer (pH=7.4) and purified LC (adjusted to produce 10-30% final cleavage) was incubated at 37° C. for 15-180 minutes. The reaction was stopped with 0.09 ml of 0.7% trifluoroacetic acid. Quantitation of cleaved and uncleaved peptide was done by reverse-phase HPLC separation and the fraction of the peptide proteolyzed was calculated by dividing the combined areas of the two cleaved peaks by the sum of the two product and uncleaved substrate peaks.

Catalytic Activity of the Expressed Constructs

Incubation of the 17-mer synthetic peptide representing residues 187-203 of SNAP-25 with the purified Lc at 37° C. generates only two peptides cleaving between residues 197 (glutamine) and 198 (arginine). No other peptide fragments were generated by prolonged incubation, indicating that any contaminants in the Lc preparation lacked proteolytic activity. FPLC purification run #71, which was the complete Lc, resulted in a specific activity of 2.36 μmol/min/mg of Lc. Native BoNT/A in previous assays with the SNAP-25 synthetic peptide had a specific activity of 0.241 μmol/min/mg (Schmidt and Bostian). Thus, the purified Lc produced had a specific activity increased by approximately 10-fold. Run #32 was the Lc+Belt, and had an activity of 0.08 μmol/min/mg.

### Example 26

Determination of the Length of the Purified Whole and Truncated Lc

HPLC-purified samples were mixed with sinapinic acid and deposited on a stainless steel target. Mass spectra were acquired with a Perseptive Biosystems Voyager DE MALDI-TOF system. Data were obtained in delayed extration mode (750 ns delay) with a 337 nm nitrogen laser (3 ns wide pulse), using an acquisition rate of 2 GHz, 50,000 channels, an accelerating voltage of 25000, 93% grid voltage, and a 0.3% guide wire voltage. Typically, 128 scans were averaged. The mass spectrometer was externally calibrated with myoglobin and bovine serum albumin.

The amino-terminal sequence of the expressed Lc was determined by automated Edman degradation performed on an Applied Biosystems Procise Sequencer (Applied Biosystems, Foster City, Calif.) in the 0-20 picomole detection range.

Determination of the Cleavage Point for Purified Lc

Purified Lc kept at −20° C. in purification buffer with 2 mM EDTA had no observable cleavage or truncation products. When the same product was placed at 30° C. for 1 hour, the truncated Lc seen after the first cation exchange column passage was observed. In a mass spectrum for cleaved BoNT/A Lc, the ion at m/z 49039.0 corresponds to the singly-charged molecule, whereas ions at m/z 24,556.9, and 98,280 correspond to doubly-charged and dimer species, respectively. The verified amino terminus for the Lc was VQFVNKQFNY (residues 2 to 11 of SEQ ID NO:5), with the terminal methione removed, resulting in a peptide of 448 residues. The observed principal mass of 49,039 is approximately 2279 daltons less than the calculated mass for type A Lc, which represents a loss of 21-22 amino acids. Since the amino terminus specific antibody still reacts with the truncated molecule, cleavage occurred near the C terminus of the molecule. Because of mass uncertainty with MALDI-TOFMS (0.05% maximum mass accuracy for this instrument), it was not possible to positively identify the site of cleavage. Nevertheless, it was determined that cleavage occurred at either Y₄₂₆, K₄₂₇, or L₄₂₈. The most probable site of cleavage was between K₄₂₇ and L₄₂₈. Calculated mass for that product was 48,999, a difference of 40 daltons, which represents the best match to the observed ion and a mass accuracy to within 0.08%.

Addition of MgCl₂ to 125 mM and incubation for 1 hour at 30° C. resulted in two cleavage products after the Lc had lost the carboxy terminal residues. Amino terminus sequencing showed the cleavage to be between two tyrosines, Y₂₅₀ and Y₂₅₁.

### Example 27

Phosphorylation of Purified Lc

Phosphorylation was at 30° C. for 1 to 24 hours in a final reaction volume of 40 μL with 30 units c-src kinase. Non-phosphorylated samples were those in which enzyme was omitted. The amount of Lc in the reaction was from 6.25 nM to 1.25 nM. The 4× buffer used for the reaction consisted of 100 mM Tris-HCl, pH 7.2, 125 mM MgClz, 25 mM MnC 12, 2 mM EGTA, and 2 mM DTT. ATP was at either 500 μM or 1 mM, with [(−³²P]ATP added to a final concentration of 1 μCi/ul. In some cases, substrate peptide (KVEKIGEGTGVVYK; SEQ ID NO:3) at 93 μM was substituted for the Lc to act as a control. Reactions were stopped by freezing at −20° C. Phosphorylated samples were run on SDS-PAGE gels, and either blotted and bands visualized with an antibody specific to phosphorylated tyrosine or the amino terminus of the Lc, or they were stained with Coomassie Blue, destained, dried and exposed to Kodak BioMax Light film.

Phosphorylation of Lc

Purified Lc that was tyrosine phosphorylated resisted cleavage at the Y₂₅₀-Y₂₅₁ site. During the initial 1 hour period of phosphorylation, the characteristic cleavage products were observed, but did not substantially increase over a 24 hour period of time. A possible explanation is that phosphorylated Lc molecules were protected from cleavage, but not all of them could be modified prior to concurrent proteolysis. An identical reaction mixture lacking the enzyme showed rapid cleavage of the Lc, with very little remaining by 4 hours, and undetectable by 8 hours. A monoclonal antibody to phosphorylated tyrosine reacted to full length, src phosphorylated Lc, but not to either of the cleavage products in the phosphorylation reaction, even though cleavage products were clearly visible by SDS-PAGE at all time points. The reaction lacking the enzyme showed no phosphorylated tyrosine bands of any size. Antibody to the amino terminus of the Lc reacted to the full length and larger of the cleavage products, plus three additional bands of between 60 and 75 kDa. These additional bands above the Lc were observed by SDS-PAGE for all the samples and appear to be SDS-resistant complexes of either the Lc or amino terminus fragment with other fragments. Autoradiographs of the phosphorylated and unphosphorylated (lacking enzyme) Lc show incorporation of [γ-³²P]ATP in the src phosphorylated full length Lc at 1 hour, with none observed in smaller or larger fragments, nor in samples lacking the enzyme. At 24 hours, very faint bands corresponding to the cleavage products did appear. These could either have arisen from cleaved, phosphorylated, full length Lc, or they may have been phosphorylated after they became fragments.

### Example 28

Immunity

Immunization of mice with the purified forms of the LcA, LcA+Belt and LcA+Hn resulted in ELISA titers of between X and X for all construct forms. Protection was observed after challenge with 10² to 10³ MLD₅₀ of purified Type A toxin. See Tables 6-8.

TABLE 6
Efficacy of Purified rBoNTA(LC + Belt) Solubly Expressed
from E. coli to Elicit Protective Immunity in Mice

| | | |
|---|---|---|
| | Toxin Challenge | |
| Dosage ^{a, b} | (Survivors/Total) | ELISA Titer |

| (μg) | 10² LD₅₀ | 10³ LD₅₀ | (GMT) ^{c} |
|---|---|---|---|
| 5 | 10/10 | 10/10 | ND |
| 15 | 10/10 | 10/10 | ND |
| Controls | 0/10 | 0/10 | ND |

^{a} Animals were vaccinated at 0, 2, and 4 weeks and challenged on week 6.
^{b} Specific activity (i.e., proteolytic activity) of the rBoNTA(LC + Belt) immunogen was not determined.
^{c} Geometric mean of the ELISA titer to BoNTA neurotoxin (ND = not determined).

TABLE 7
Efficacy of Purified rBoNTA(LC + Hn) Solubly Expressed
from E. coli to Elicit Protective Immunity in Mice

| | | |
|---|---|---|
| | Toxin Challenge | |
| Dosage ^{a, b} | (Survivors/Total) | ELISA Titer |

| (μg) | 10² LD₅₀ | 10³ LD₅₀ | (GMT) ^{c} |
|---|---|---|---|
| 5 | 5/9 | 1/9 | ND |
| 15 | 4/10 | 1/10 | ND |
| Controls | 0/10 | 0/10 | ND |

^{a} Animals were vaccinated at 0, 2, and 4 weeks and challenged on week 6.
^{b} Specific activity (i.e., proteolytic activity) of the rBoNTA(LC + Hₙ) immunogen was not determined.
^{c} Geometric mean of the ELISA titer to BoNTA neurotoxin (ND = not determined).

TABLE 8
Efficacy of Purified rBoNTA(LC) Solubly Expressed
from E. coli to Elicit Protective Immunity in Mice

| | | |
|---|---|---|
| | Toxin Challenge | |
| Dosage ^{a, b} | (Survivors/Total) | ELISA Titer |

| (μg) | 10² LD₅₀ | 10³ LD₅₀ | (GMT) ^{c} |
|---|---|---|---|
| 5 | 9/10 | 10/10 | ND |
| 15 | 9/10 | 10/10 | ND |
| Controls | 0/10 | 0/10 | ND |

^{a} Animals were vaccinated at 0, 2, and 4 weeks and challenged on week 6.
^{b} Specific activity of the rBoNTB(LC) immunogen was 21 mmol/min/mg using 0.8-1.0 mM substrate (VAMP peptide, residues 54-94).
^{c} Geometric mean of the ELISA titer to BoNTB neurotoxin (ND = not determined).

### Example 29

Discussion

The system of expression of the invention for botulinum neurotoxin Hc (Byrne et al, 1998) and Lc fragments using an optimized synthetic gene, has previously shown success in achieving high levels of product. In an attempt to produce a molecule that more closely resembles the natural state of the toxin, a cloning and expression scheme that would give a large amount of correctly folded, untagged, Lc was initiated. The two basic strategies employed were to (1) express the Lc at a lower temperature, a classic method for ensuring proper folding, and (2) adding on portions of the rest of the neurotoxin polypeptide, mimicking the natural expression within the clostridial host. As expected, reducing the temperature for induction dramatically increased the solubility of the expressed product from 5.2% at 37° C. to 55.5% at 18° C. for the Lc. The slower rate of expression at the lower temperatures was compensated for by increasing the length of time for expression. This did not result in increased degradation of the product intracellularly, prior to harvest and purification. Addition of the belt and Hn portions of the toxin had no effect upon solubility of the expressed gene, although each was easily expressed at the lower temperature.

Although cloned and expressed Lc has been available for Lc study, it has been purified with either glutathione or his-tags (Zhou, et al, 1995; Li and Singh, 1999). Previous investigators have used native toxin (Lacy et al, 1998) for x-ray crystallography studies, and it was an object of the invention to produce Lc as close to the native product as possible, e.g., without tags or modifications. For this reason, traditional column chromatography methods were used instead of affinity columns. The calculated pI of the Lc of 8.13 suggested that the Lc would efficiently bind to a cation exchange column. Upon passage over an initial Mono S column, the product appeared relatively clean, although a second immunoreactive band immediately beneath the proper, calculated size for the Lc was noted. After passage over a second cationic exchange column, this band was not observed on Westerns.

Using the above methods of low temperature expression and cation exchange purification, a large quantity of Lc was acquired for assessment of catalytic activity. Activity of the purified Lc was calculated to be approximately 10-fold greater than that of the native toxin. Previous investigators have shown that the Lc must be activated by proteolytic cleavage of the Lc from the Hc (DasGupta and Dekleva, 1990), although the two halves must both be present for efficient intoxication of cells. It is interesting that the Lc with the belt attached lacked the high level of catalytic activity seen with the Lc by itself. Presumably, the belt is wrapped around the Lc, as is observed in x-ray crystallography studies (Lacy et al, 1998). As the entire translocation region is not there to occlude the active site, it may be that the belt in some manner is constricting the Lc, or a conformational change is prevented that is required for full activation. Comparison of the crystallography structure of Lc of the invention with and without the belt would be worth further study.

Two interesting and unexpected pieces of data came from expression of Lc without purification tags. The first was the truncation of the Lc from the carboxy terminus by 20 residues. A recent paper by Kadkhodayan et al, 2000, notes that this portion of the Lc is not required for full catalytic activity. The truncation is intriguing as it removes the Lc/Hc di-sulfide bond at a lysine proximal to the involved cysteine. The two other proteolytic cleavages known to occur at the carboxy terminus of the Lc are also at lysine residues (DasGupta and Dekleva, 1990). Lysine proteolysis is common, with ubiquitin, a lysine specific proteolysis factor found conjugated to cell receptors of eukaryotes being one of the most common routes (Doherty and Mayer, 1992). It has long been hypothesized that the di-sulfide bond holding the Lc and Hc together was reduced as the Lc was transported into the cell, freeing it from the receptor binding portion (de Paiva et al, 1993). Although the ten residue portion flanked by lysine residues seems to be removed during activation “nicking” of the polypeptide, the cysteine residue was assumed to remain as part of the Lc. Work with native toxin and cells has been initiated to determine if the natural state of the toxin inside cells is one lacking the terminal 20 residues and cysteine.

### Example 30

Expression of BoNT LC

Reagents: Terrific Broth (Difco): 48 gm/liter with 4 ml of non-animal glycerol; autoclave 15 minutes. Store refrigerated. Kanamycin: stock solution is 50 mg/ml in distilled water, filter sterilized, store in aliquots at −20° C. Chloramphenicol: stock solution is 50 mg/ml in ethanol, filter sterilized, store in aliquots at −20° C. Add antibiotics to media just prior to use.

Expression of the Lc and Lc with Hc (translocation region) was performed for even numbered SEQ ID NOS:20-44. Expression was essentially the same for all constructs within the given parameters.

Cultures of BL21(DE3) cells were grown in Terrific Broth (TB) plus 50 μg/mL kanamycin. Cultures of BL21(DE3) Codon Plus cells were grown in TB plus 50 μg/mL kanamycin and 50 μg/mL chloramphenicol. Cultures grown overnight at 37° C. while shaking at about 200 to about 250 rpm were diluted 1:20 with fresh antibiotic-containing media. Diluted cultures were returned to overnight growth conditions (37° C., shaking at 200-250 rpm) for 1¼ to 2½ hours. An optical density measurement was taken while the cultures were placed on ice for 5 minutes. Preferably, the OD₆₀₀ is between about 0.4 and about 0.6. The incubation time may be extended and/or fresh antibiotic-containing media may be added if the OD₆₀₀ is lower than 0.4 or higher than 0.6.

Next, sufficient IPTG was added to each chilled culture to make the concentration about 1 mM. IPTG-containing cultures were incubated about 24 to about 26 hours at 18° C. and shaking at about 200 to about 250 rpm. An optical density measurement was taken at the end of this incubation. Preferably, the OD₆₀₀ is between about 1.7 and about 2.1.

Cultures that satisfied this criteria were centrifuged at about 3000 rpm for about 20 minutes to obtain a cell paste for purification. The cell paste may be stored at −20° C. until ready for use.

Aliquots of 1 mL each were pelleted in a microfuge, resuspended in 1 mL of sonication buffer, and sonicated 12×30 seconds on ice over 12 minutes. Sonicated cells were microfuged for 10 minutes. The supernatant was aspirated and retained as the soluble fraction. 1 mL of 6M urea was added to each pellet and retained as the insoluble fraction. Appropriate amounts run on by SDS-PAGE should show approximately 50% soluble, 50% insoluble, at about 51 kDa. A western with rabbit anti-Lc sera will be at the same location.

Purification of BoNT LC

Cell paste was resuspended at 1 g/20 mL sonication buffer, sonicated 10×, 30 seconds on, 30 seconds off, on ice. Insoluble material and debris was pelleted by centrifuging for 10 minutes at 12,000 rpm (e.g. in a microfuge), decanting solute, and repeating one time in a fresh tube. The supernatant was decanted into a fresh tube. An equal volume of equilibration buffer may be optionally added to the supernatant to facilitate cation exchange chromatography, e.g., flow. For example, such dilution facilitates column loading and washing when using a Source S resin from Pharmacia whereas such dilution is unnecessary when using a Poros cationic resin. Filter sterilize the supernatant with 0.45 μm filters.

Run #1: A column (100 mm) was equilibrated with equilibration buffer, 2 minutes, 2.5 to 3 ml/min (same rate through out run). Cell paste (20-40 mL per run) was manually loaded. The column was washed for 3 minutes with equilibration buffer. Using gradient buffer, a 0 to 70% gradient was run over 8 minutes. For some cell lysates, a 5% NaCl (5 mS) 5 minutes step was performed. For example, where a Source S resin was used, no salt wash was was performed, but where a Poros resin was used, this salt wash was performed to elute contaminating proteins. Cell protein was collected at between 10 and 22 mS. Fractions (1 mL) were collected through out the gradient. The desired protein will elute at between 10 and 22 mS, depending upon the expression product used.

Run#2: The peak fractions from run #1 were pooled. Equilibration buffer was added to pooled fractions, at a 3:1 ratio. The column was equilibrated with equilibration buffer for 2 minutes, at 2.5 to 3 ml/min (same rate through out run). The run#1 pool was loaded onto the column; washed 2 minutes with equilibration buffer. Using gradient buffer, a 0 to 75% gradient was run over 15 minutes. Fractions (1 mL) were collected and peak fractions were pooled. Aliquots of the pooled fractions were stored in plastic vials at −20° C.

A portion of the purified protein was used to measure the A_{260/278}. The ratio may be used as a measure of the presence of DNA and the A₂₈₀ to quantitate the protein by using the calculated molar extinction coefficient and molecular weight.

A cleaning procedure must be done on the column between each run. Run 1 M NaCl through column at 3 ml/min for 5 minutes. Run 20 mM NaOH through the column at 1 ml/min for 10 minutes. Run 70% ETOH through the column at 1 ml/min for 30 minutes. Run 1 M NaCl through it at 1 ml/min for 15 minutes. Re-equilibrate the column to the proper pH with a low salt buffer.

Buffers

A combination of sonication buffers, equilibration buffers and gradient buffers is used for each cell lysate. Sonication buffers are always chosen to be 0.4 pH below the equilibration buffer. Gradient buffers are the same as equilibration buffers except for addition of 1 M NaCl.

Gradient buffer A: 55 mM Na mono-phosphate, 2 mM EDTA, 1 M NaCl, in milliQ water; pH to 5.8; filter. Gradient buffer B: 20 mM NaAcetate, 1 M NaCl, in milliQ water, pH to 5.4, filter. Gradient buffer C1: 20 mM NaAcetate, 1 M NaCl, in milliQ water, pH to 4.8, filter. Gradient buffer C2: 20 mM NaAcetate, 2 mM EDTA, 1 M NaCl, in milliQ water, pH to 5.4, filter. Gradient buffer D: 20 mM NaAcetate, 2 mM EDTA, 1 M NaCl, in milliQ water, pH to 4.8, filter.

Results

Expression and purification of BoNT/A LC according to this method yielded protein with a specific activity (SNAP-25 assay) that was about 10-fold higher than when BoNT/A LC was purified from inclusion bodies (Ahmed and Smith (2000) J. Prot Chem. 19, 475-487).

### REFERENCES

The references cited throughout this application and listed below are incorporated herein in their entirety by reference.
Adler, M., Dinternan, R. E., and Wannemacher, R. W. (1997). Toxicon 35, 1089-1110.
Ahmed, S. A. and Claiborne, A. (1992). J. Biol. Chem. 267, 3822-3840.
Ahmed, S. A. and Smith, L. A. (2000). J. Protein Chem. 19, 475-487.
Ahmed, S. A., Byrne, M. P., Jensen, M., Hines, H. B., Brueggemann, E., and Smith, L. A. (2001). J. Protein Chem. 20, 221-231.
Ahmed, S. A., Fairwell, T., Dunn, S., Kirschner, K., and Miles, E. W. (1986). Biochemistry 25, 3118-3124.
Alderton, J. M., Ahmed, S. A., Smith, L. A., and Steinhardt, R. A. (2000). Cell. Calcium 28, 161-169.
Andersson, S. G., and Kurland, C. G. (1990). Microbial. Rev. 54, 198-210.
Auld, D. S. (1995). Meth. EnUmol. 248, 228-242.
Bi, G. Q., Alderton, J. M., and Steinhardt, R. A. (1995). J. Cell Biol. 131, 1747-1758.
Bittner, M. A., DasGupta, B. R., and Holz, R. W. (1989). J. Biol. Chem. 264, 10354-10360.
Black, J. D., and Dolly, J. O. (1986). J. Cell Biol. 103, 535-544.
Blasi, J., Chapman, E. R., Link, E., Binz, T., Yamasaki, S., De Camilli, P., Sudhof, T. C. Niemann, H., and Jahn, R. (1993). Nature 365, 160-163.
Cai, S., Sarkar, H. K., and Singh, B. R. (1999). Biochemistry 38, 6903-6910.
Cardoso F, Jankivic J (1995). Clinical use of botulinum neurotoxins. In Current Topics in Microbiology and Immunology (Capron A et al., eds.), Springer-Verlag, Germany, pp. 123-141.
Chen, F., Kuziemko, G. M., Amersdorfer, P., Wong, C., Marks, J. D., and Stevens, R. C. (1997). Infect. Immun. 65, 1626-1630.
Claiborne, A., Yeh, J. I., Mallett, T. C., Luba, J., Crane, E. J., 3rd, Charrier, V., and Parsonage, D. (1999). Biochemistry 38, 15407-15416.
Creighton, T. E. (1984). Proteins, Structures and Molecular Properties, Freeman, N.Y.
Dalbey, R. E. and Kahn, A. (2000). Annu. Rev. Cell Dev. Biol. 16, 51-87.
DasGupta, B. R., and Dekleva, M. L. (1990). Biochimie 72, 661-664.
DasGupta, B. R., and Foley, J., Jr. (1989). Biochimie 71, 1193-1200.
Dekleva, M. L. and DasGupta, B. R. (1990). J. Bacteriol. 172, 2498-2503.
de Paiva, A., Poulain, B., Lawrence, G. W., Shone, C. C., Tauc, L., and Dolly, J. O. (1993). J. Biol. Chem. 268, 20838-20844.
Dertzbaugh, M. T., and West, M. W. (1996). Vaccine 14, 1538-1544.
Ettinger, R. A., Liu, A. W., Nepom, G. T., and Kwok, W. W. (2000). J. Immunol 165, 3232-3238.
Foran, P., Shone, C. C., and Dolly, J. O. (1994). Biochemistry 33, 15365-15374.
Foran, P., Lawrence, G. W., Shone, C. C., Foster, K. A., and Dolly, J. O. (1996). Biochemistry 35, 2630-2636.
Fu, F. N., Lomneth, R. B., Cai, S., and Singh, B. R. (1998). Biochemistry 37, 5267-5278.
Kadkhodayan, S., Knapp, M. S., Schmidt, J. J., Fabes, S. E., Rupp, B., and Balhorn, R. (2000). Protein Expr. Purif 19, 125-130.
Kiyatkin, N., Maksymowych, A. B., and Simpson, L. L. (1997). Infect. Immun. 65, 4586-4591.
Klatt, P., Schmidt, K., Lehner, D., Glatter, O., Bachinger, H. P., and Mayer, B. (1995). EMBO J. 14, 3687-3695.
Knapp, M., Segelke, B., Balhorn, R., and Rupp. B. (2000). The crystal structure of botulinum toxin A zinc protease domain. Presented at the 37th Annual Meeting of the Interagency Botulinum Research Coordinating Committee, Alisomar, Calif.
Kreiglstein, K. G., DasGupta, B. R., and Henschen, A. H. (1994). J. Protein Chem. 13, 49-57.
Kurazono, H. Mochida, S., Binz, T., Eisel, U., Quanz, M., Grebenstein, O., Wetnars, K., Poulain, B., Tauc, L., and Niemann, H. (1992). J. Biol. Chem. 267, 14721-14729.
Lacy, D. B., and Stevens, R. C. (1999). J. Mol. Biol. 291, 1091-1104.
Lacy, D. B., Tepp, W., Cohen, A. C., DasGupta, B. R., and Stevens, R. C. (1998). Nature Struct. Biol. 5, 898-902.
Laemmli, U. K. (1970). Nature 227, 680-685.
Lebeda, F. J., and Olson, M. A. (1994). Proteins 20, 293-300.
Li, L., and Singh, B. R. (1999). Protein Expr. Purif. 17, 339-344.
Li, L. and Singh, B. R. (2000). Biochemistry 39, 10581-10586.
Li, Y., Foran. P., Fairweather, N. F., de Paiva, A., Weller, U., Dougan, G., and Dolly, J. O. (1994). Biochemistry 33, 7014-7020.
Maisey, E. A., Wadsworth, J. D., Poulain, B., Shone, C. C., Melling. J. Gibbs, P., Tauc, L., and Dolly, J. O. (1988). Eur. J. Biochem. 177, 683-691.
Makoff, A. J., Oxer, M. D., Romanos, M. A., Fairweather, N. F., and Ballantine, S. (1989). Nucleic Acids Res. 17, 10191-10202.
Montal, M. S., Blewitt, R., Tomich, J. M., and Mortal, M. (1992). FEBS Lett. 313, 12-18.
Montecucco, C., and Schiavo, G. (1994). Mol. Microbiol 13, 1-8.
Montecucco, C., and Schiavo, G. (1995). Q. Rev. Biophys. 28, 423-472.
Nowakowski, J. L., Courtney, B. C., Bing, Q. A., and Adler, M. (1998). J. Protein Chem. 17, 453-462.
Pace, C. N., Vajdos, F., Fee, L., Grimsley, G., and Gray, T. (1995). Protein Sci. 4, 2411-2423.
Rossetto, O., Schiavo, G., Montecucco, C., Poulain, B., Deloye, F. Lozzi, L., and Shone, C. C. (1994). Nature 372, 415-416.
Schiavo, G., Rossetto, O., Catsicas, S., Polyerino de Laureto, P., DasGupta, B. R., Benfenati, F., and Montecucco, C. (1993). J. Biol. Chem. 268, 23784-23787.
Schiavo, G., Malizio, C., Trimble, W. S., Polyerino de Laureto, P. Milan, G., Sugiyama, H., Johnson, E. A., and Montecucco, C. (1994). J. Biol. Chem. 269, 20213-20216.
Schiavo, G. Rossetto, Tonello, F., and Montecucco, C. (1995). Intracellular targets and metalloprotease activity of tetanus and botulinum neurotoxins. In Clostridial Neurotoxins: The Molecular Pathogenesis of Tetanus and Botulism (Montecucco, C., ed.), Springer, N.Y., pp. 257-273.
Schmidt, J. J., and Bostian. K. A. (1995). J. Protein Chem. 14, 703-708.
Schmidt, J. J., and Bostian. K. A. (1997). J. Protein Chem. 16, 19-26.
Schmidt, J. J., Stafford R G, Millard C B (2001). Analytical Biochemistry 296, 130-137.
Shone, C. C., and Roberts, A. K. (1994). Eur. J. Biochem. 225, 263-270.
Shone, C. C., and Tranter, H. S. (1995). Curr. Top. Microbiol. Immunol. 195, 143-160.
Shone, C. C., Quinn, C. P., Wait, R., Hallis, B., Fooks, S. G., and Hambleton, P. (1993). Eur. J. Biochem. 217, 965-971.
Schagger, H. and von Jagow, G. (1987). Anal. Biochem. 166, 368-379.
Schmidt, J. J., Stafford, R. G., and Bostian, K. A. (1998). FEBS Lett. 435, 61-64.
Sheridan, R. E., Deshpande, S. S., Nicholson, J. D., and Adler, M. (1997). Toxicon 35, 1439-1451.
Simpson, L. L., Coffield, J. A., and Bakry, N. (1993). J. Pharmacol. Exp. Ther. 267, 720-727.
Smith, L. A. (1998). Toxicon 36, 1539-1548.
Steinhardt, R. A., Bi, G., and Alderton, J. M. (1994). Science 263, 390-393.
Strasser, A., O'Connor, L., and Dixit, V. M. (2000). Annu. Rev. Biochem 69, 217-245.
Syuto, B., and Kubo, S. (1981). J. Biol. Chem. 256, 3712-3717.
Thompson, D. E., Brehm, J. K., Oultram, J. D., Swinfield, T. J., Shone, C. C. Atkinson, T., Melling, J., and Minton, N. P. (1990). Eur. J. Biochem. 189, 73-81.
Washbourne, P., Pellizzari, R. Baldini, G., Wilson, M. C., and Montecucco, C. (1997). FEBS Lett. 418, 1-5.
Winkler, H. H., and Wood, D. O. (1988). Biochimie 70, 977-986.
Zhou, L., de Paiva, A., Liu, D., Aoki, R., and Dolly, J. O. (1995). Biochemistry 34, 15175-15181.

I claim:

1. A method for producing a botulinum neurotoxin light chain comprising:
culturing a host cell comprising a DNA molecule encoding the botulinum neurotoxin light chain, the DNA molecule having a nucleotide sequence expressible in the host cell, at a temperature below 30° C., wherein the DNA molecule is expressed and the light chain is produced, and
isolating the botulinum neurotoxin light chain.

2. A method for producing a botulinum neurotoxin light chain comprising:
culturing a host cell comprising a DNA molecule encoding the botulinum neurotoxin light chain, the DNA molecule having a nucleic acid sequence expressible in the host cell, at about 18° C., wherein DNA molecule is expressed and the botulinum neurotoxin light chain is produced, and
isolating the botulinum neurotoxin light chain.

3. The method of claim 1 or 2 wherein the host cell is selected from the group consisting of Escherichia coli and Pichia pastoris.

4. The method of claim 1 or 2 wherein the host cell is Escherichia coli.

5. The method of claim 1 or 2 further comprising obtaining an insoluble protein fraction from the cultured host cell and purifying the botulinum neurotoxin light chain from the insoluble protein fraction.

6. The method of claim 5 further comprising solubilizing the insoluble protein fraction.

7. The method of claim 6 further comprising subjecting the solubilized insoluble protein fraction to cation exchange chromatography under conditions such that a purified preparation of botulinum neurotoxin light chain is obtained.

8. The method of claim 1 or 2 further comprising obtaining a soluble protein fraction from the cultured host cell and purifying the botulinum neurotoxin light chain from the soluble protein fraction.

9. The method of claim 8 further comprising subjecting the soluble protein fraction to cation exchange chromatography under conditions such that a purified preparation of botulinum neurotoxin light chain is obtained.

10. The method of claim 1 or 2 wherein more than about 100 mg of purified botulinum neurotoxin light chain is obtained per liter of culture.

11. The method of claim 10 wherein more than 500 mg of purified botulinum neurotoxin light chain is obtained per liter of culture.

12. The method of claim 11 wherein about 1 gram of purified botulinum neurotoxin light chain is obtained per liter of culture.

13. The method of claim 1 or 2 wherein the purified botulinum neurotoxin light chain is catalytically active.

14. The method of claim 1 or 2 wherein the DNA Molecules has the nucleic acid sequence of nucleotides 9-1337 of SEQ ID NO:4.

15. The method of claim 1 or 2 wherein the DNA molecules encodes a botulinum neurotoxin light chain serotype A.

16. The method of claim 1 or 2 wherein the DNA molecule encodes a botulinum neurotoxin light chain selected from the group consisting of botulinum neurotoxin light chain serotype B, botulinum neurotoxin light chain serotype C₁, botulinum neurotoxin light chain serotype D, botulinum neurotoxin light chain serotype E, botulinum neurotoxin light chain serotype F, and botulinum neurotoxin light chain serotype G.

17. The method of claim 15 wherein the nucleic acid has a total A+T content that is less than about 70%.

18. The method of claim 17 wherein the nucleic acid molecule encodes a polypeptide having an amino acid sequence selected from the group consisting of SEQ ID NO:47 and SEQ ID NO: 21.

19. The method of claim 17 wherein the A+T content of any 50 consecutive nucleotides of the nucleic acid molecule is less than about 75%.

20. The method of claim 18 wherein the A+T content of any 50 consecutive nucleotides of the nucleic acid molecule is less than about 75%.

21. The method of claim 16 wherein the nucleic acid molecule has a a nucleic acid sequence selected from the group consisting of SEQ ID NOS:6, 8, 10, 12, 14, 16, 22, 26, 30, 34, 38, and 42.

22. The method of claim 16 wherein the nucleic acid has a total A+T content that is less than about 70%.

23. The method of claim 22 wherein the A+T content of any 50 consecutive nucleotides of the nucleic acid molecule is less than about 75%.

24. The method of claim 22 wherein the nucleic acid molecule encodes a polypeptide having an amino acid sequence selected from the group consisting of SEQ ID NO:7, SEQ ID NO:9, SEQ ID NO:11, SEQ ID NO:13, SEQ ID NO:15, SEQ ID NO:17, SEQ ID NO:23, SEQ ID NO:27, SEQ ID NO:31, SEQ ID NO:35, SEQ ID NO:39, and SEQ ID NO:43, wherein the nucleic acid has a total A+T content that is less than about 70%.
